SEQUENCE LISTING (221 sequences)

SEQ ID NO: 1 - protein, 49 aa, Artificial Sequence
  Synthetic
MIPGGLSEAKPATPEIQEIVDKVKPQLEEKTNETYGKLEAVQYKTQVLA

SEQ ID NO: 2 - protein, 24 aa, Artificial Sequence
  Synthetic
GTNYYIKVRAGDNKYMHLKVFKSL

SEQ ID NO: 3 - protein, 21 aa, Artificial Sequence
  Synthetic
EDLVLTGYQVDKNKDDELTGF

SEQ ID NO: 4 - protein, 134 aa, Artificial Sequence
  Synthetic
  misc_feature (4)..(4): Xaa may be Gly, Ala, Val, Arg, Lys, Asp, or Glu
  misc_feature (50)..(69): Xaa may be any naturally-occuring amino acid
  misc_feature (53)..(69): may be absent
  misc_feature (70)..(70): Xaa may be Gly, Ala, Val, Ser or Thr
  misc_feature (91)..(91): Xaa may be Arg, Lys, Asn, Gln, Ser, Thr
  misc_feature (92)..(92): Xaa may be Gly, Ala, Val, Ser or Thr
  misc_feature (93)..(93): Xaa may be Ala, Val, Ile, Leu, Gly or Pro
  misc_feature (94)..(113): Xaa may be any naturally-occuring amino acid
  misc_feature (97)..(113): may be absent
  misc_feature (114)..(114): Xaa may be Gly, Ala, Val, Asp or Glu
  misc_feature (116)..(116): Xaa may be Ala, Val, Ile, Leu, Arg or Lys
MIPXGLSEAKPATPEIQEIVDKVKPQLEEKTNETYGKLEAVQYKTQVLAXXXXXXXXXXXXXXXXXXXXXTNYYIKVRAGDNKYMHLKVFXXXXXXXXXXXXXXXXXXXXXXXXDXVLTGYQVDKNKDDELTGF

SEQ ID NO: 5 - protein, 134 aa, Artificial Sequence
  Synthetic
  misc_feature (50)..(69): Xaa may be any naturally-occuring amino acid
  misc_feature (53)..(69): may be absent
  misc_feature (94)..(113): Xaa may be any naturally-occuring amino acid
  misc_feature (97)..(113): may be absent
MIPRGLSEAKPATPEIQEIVDKVKPQLEEKTNETYGKLEAVQYKTQVLAXXXXXXXXXXXXXXXXXXXXSTNYYIKVRAGDNKYMHLKVFNGPXXXXXXXXXXXXXXXXXXXXADRVLTGYQVDKNKDDELTGF

SEQ ID NO: 6 - protein, 9 aa, Artificial Sequence
  Synthetic
HDQDVLHAW

SEQ ID NO: 7 - protein, 9 aa, Artificial Sequence
  Synthetic
KFHRQEWAD

SEQ ID NO: 8 - protein, 9 aa, Artificial Sequence
  Synthetic
PEDFWDPEH

SEQ ID NO: 9 - protein, 9 aa, Artificial Sequence
  Synthetic
VVRTTGHVV

SEQ ID NO: 10 - protein, 9 aa, Artificial Sequence
  Synthetic
YWWFCTGQS

SEQ ID NO: 11 - protein, 9 aa, Artificial Sequence
  Synthetic
IHHRQARSL

SEQ ID NO: 12 - protein, 9 aa, Artificial Sequence
  Synthetic
SHRRRAYIW

SEQ ID NO: 13 - protein, 9 aa, Artificial Sequence
  Synthetic
WDSHHWRAP

SEQ ID NO: 14 - protein, 8 aa, Artificial Sequence
  Synthetic
DKRVKYGQ

SEQ ID NO: 15 - protein, 9 aa, Artificial Sequence
  Synthetic
SDWVYALQL

SEQ ID NO: 16 - protein, 6 aa, Artificial Sequence
  Synthetic
FWWFWY

SEQ ID NO: 17 - protein, 9 aa, Artificial Sequence
  Synthetic
VRDWPWNTF

SEQ ID NO: 18 - protein, 9 aa, Artificial Sequence
  Synthetic
QKKRDEDYI

SEQ ID NO: 19 - protein, 9 aa, Artificial Sequence
  Synthetic
GVHEEPRKL

SEQ ID NO: 20 - protein, 9 aa, Artificial Sequence
  Synthetic
EWWQKHWPS

SEQ ID NO: 21 - protein, 9 aa, Artificial Sequence
  Synthetic
NFFQRRWPG

SEQ ID NO: 22 - protein, 9 aa, Artificial Sequence
  Synthetic
DWWQAKWPH

SEQ ID NO: 23 - protein, 9 aa, Artificial Sequence
  Synthetic
GIWQSRWPG

SEQ ID NO: 24 - protein, 9 aa, Artificial Sequence
  Synthetic
GYWAAKWPG

SEQ ID NO: 25 - protein, 9 aa, Artificial Sequence
  Synthetic
GFYADHWPG

SEQ ID NO: 26 - protein, 9 aa, Artificial Sequence
  Synthetic
NWYQQRWPG

SEQ ID NO: 27 - protein, 9 aa, Artificial Sequence
  Synthetic
GFYARHWPG

SEQ ID NO: 28 - protein, 9 aa, Artificial Sequence
  Synthetic
DFWKAHWPG

SEQ ID NO: 29 - protein, 9 aa, Artificial Sequence
  Synthetic
DFYSVRWPG

SEQ ID NO: 30 - protein, 9 aa, Artificial Sequence
  Synthetic
YWAANHASK

SEQ ID NO: 31 - protein, 9 aa, Artificial Sequence
  Synthetic
IKRLEHWEY

SEQ ID NO: 32 - protein, 9 aa, Artificial Sequence
  Synthetic
EWDSPWSEN

SEQ ID NO: 33 - protein, 9 aa, Artificial Sequence
  Synthetic
KHKNLRWPF

SEQ ID NO: 34 - protein, 9 aa, Artificial Sequence
  Synthetic
RHFPKQTNW

SEQ ID NO: 35 - protein, 9 aa, Artificial Sequence
  Synthetic
VWGPEYQHQ

SEQ ID NO: 36 - protein, 9 aa, Artificial Sequence
  Synthetic
TWKNNGQDV

SEQ ID NO: 37 - protein, 9 aa, Artificial Sequence
  Synthetic
ATWLNYYLP

SEQ ID NO: 38 - protein, 9 aa, Artificial Sequence
  Synthetic
DQESLFLNN

SEQ ID NO: 39 - protein, 9 aa, Artificial Sequence
  Synthetic
GFYAQHWPD

SEQ ID NO: 40 - protein, 9 aa, Artificial Sequence
  Synthetic
GHYARYWPG

SEQ ID NO: 41 - protein, 9 aa, Artificial Sequence
  Synthetic
GFWASKWPG

SEQ ID NO: 42 - protein, 9 aa, Artificial Sequence
  Synthetic
GFWQRKWPN

SEQ ID NO: 43 - protein, 9 aa, Artificial Sequence
  Synthetic
VWPADNDLK

SEQ ID NO: 44 - protein, 9 aa, Artificial Sequence
  Synthetic
HWAWTSPGY

SEQ ID NO: 45 - protein, 9 aa, Artificial Sequence
  Synthetic
NFFQRRWPG

SEQ ID NO: 46 - protein, 9 aa, Artificial Sequence
  Synthetic
HHSHRLKGQ

SEQ ID NO: 47 - protein, 9 aa, Artificial Sequence
  Synthetic
YQNTIFLSI

SEQ ID NO: 48 - protein, 9 aa, Artificial Sequence
  Synthetic
FQDQFTWSQ

SEQ ID NO: 49 - protein, 9 aa, Artificial Sequence
  Synthetic
GEPHWPWQA

SEQ ID NO: 50 - protein, 9 aa, Artificial Sequence
  Synthetic
ADPRHPWVE

SEQ ID NO: 51 - protein, 9 aa, Artificial Sequence
  Synthetic
FHKRFQSQG

SEQ ID NO: 52 - protein, 9 aa, Artificial Sequence
  Synthetic
EWWQNRWPN

SEQ ID NO: 53 - protein, 9 aa, Artificial Sequence
  Synthetic
EWYQTRWPG

SEQ ID NO: 54 - protein, 9 aa, Artificial Sequence
  Synthetic
EFWQRHWPG

SEQ ID NO: 55 - protein, 9 aa, Artificial Sequence
  Synthetic
KFYERHWPG

SEQ ID NO: 56 - protein, 9 aa, Artificial Sequence
  Synthetic
GWWQRRWPG

SEQ ID NO: 57 - protein, 9 aa, Artificial Sequence
  Synthetic
RFKYFAHYQ

SEQ ID NO: 58 - protein, 9 aa, Artificial Sequence
  Synthetic
FHDFWHRRW

SEQ ID NO: 59 - protein, 9 aa, Artificial Sequence
  Synthetic
DWYHYWWEV

SEQ ID NO: 60 - protein, 9 aa, Artificial Sequence
  Synthetic
STRSIHVTT

SEQ ID NO: 61 - protein, 9 aa, Artificial Sequence
  Synthetic
KQHHHYLDK

SEQ ID NO: 62 - protein, 9 aa, Artificial Sequence
  Synthetic
HSAQDREIP

SEQ ID NO: 63 - protein, 9 aa, Artificial Sequence
  Synthetic
WVQSGYNSQ

SEQ ID NO: 64 - protein, 9 aa, Artificial Sequence
  Synthetic
AVFWGKWSD

SEQ ID NO: 65 - protein, 9 aa, Artificial Sequence
  Synthetic
QSFDKPWTT

SEQ ID NO: 66 - protein, 9 aa, Artificial Sequence
  Synthetic
HYPLKYSFE

SEQ ID NO: 67 - protein, 9 aa, Artificial Sequence
  Synthetic
WHHPWHRNR

SEQ ID NO: 68 - protein, 9 aa, Artificial Sequence
  Synthetic
DPWWAWVVW

SEQ ID NO: 69 - protein, 9 aa, Artificial Sequence
  Synthetic
FDNQDLIQY

SEQ ID NO: 70 - protein, 9 aa, Artificial Sequence
  Synthetic
EKKNWYKWD

SEQ ID NO: 71 - protein, 9 aa, Artificial Sequence
  Synthetic
DRHKSRWGI

SEQ ID NO: 72 - protein, 9 aa, Artificial Sequence
  Synthetic
LNPFTPSVT

SEQ ID NO: 73 - protein, 9 aa, Artificial Sequence
  Synthetic
YKGALLNHD

SEQ ID NO: 74 - protein, 9 aa, Artificial Sequence
  Synthetic
WKFRNTERG

SEQ ID NO: 75 - protein, 9 aa, Artificial Sequence
  Synthetic
YKVHQSSGG

SEQ ID NO: 76 - protein, 9 aa, Artificial Sequence
  Synthetic
FHPIAGRPW

SEQ ID NO: 77 - protein, 9 aa, Artificial Sequence
  Synthetic
FPNTSYDLQ

SEQ ID NO: 78 - protein, 9 aa, Artificial Sequence
  Synthetic
FAHYNLKSG

SEQ ID NO: 79 - protein, 9 aa, Artificial Sequence
  Synthetic
WHNYGESSG

SEQ ID NO: 80 - protein, 9 aa, Artificial Sequence
  Synthetic
KFYYADHQW

SEQ ID NO: 81 - protein, 9 aa, Artificial Sequence
  Synthetic
YTHADPHSQ

SEQ ID NO: 82 - protein, 9 aa, Artificial Sequence
  Synthetic
FGVPQLGAG

SEQ ID NO: 83 - protein, 9 aa, Artificial Sequence
  Synthetic
YSGFPFAGF

SEQ ID NO: 84 - protein, 9 aa, Artificial Sequence
  Synthetic
WFSWPYTPL

SEQ ID NO: 85 - protein, 9 aa, Artificial Sequence
  Synthetic
YYHPSIQST

SEQ ID NO: 86 - protein, 9 aa, Artificial Sequence
  Synthetic
FLGWKDTVV

SEQ ID NO: 87 - protein, 9 aa, Artificial Sequence
  Synthetic
DWWKWWWAK

SEQ ID NO: 88 - protein, 9 aa, Artificial Sequence
  Synthetic
NAGWPLVPE

SEQ ID NO: 89 - protein, 9 aa, Artificial Sequence
  Synthetic
YALDPFGGK

SEQ ID NO: 90 - protein, 9 aa, Artificial Sequence
  Synthetic
GYKFWGVSD

SEQ ID NO: 91 - protein, 9 aa, Artificial Sequence
  Synthetic
QGKQYILLR

SEQ ID NO: 92 - protein, 9 aa, Artificial Sequence
  Synthetic
YKRHSAHDY

SEQ ID NO: 93 - protein, 9 aa, Artificial Sequence
  Synthetic
WAQKSKVHQ

SEQ ID NO: 94 - protein, 9 aa, Artificial Sequence
  Synthetic
FTAVSKKDA

SEQ ID NO: 95 - protein, 9 aa, Artificial Sequence
  Synthetic
WGDKENIWF

SEQ ID NO: 96 - protein, 9 aa, Artificial Sequence
  Synthetic
WSGHPWVQK

SEQ ID NO: 97 - protein, 9 aa, Artificial Sequence
  Synthetic
YADYPLSPK

SEQ ID NO: 98 - protein, 9 aa, Artificial Sequence
  Synthetic
WKFRNTDRG

SEQ ID NO: 99 - protein, 9 aa, Artificial Sequence
  Synthetic
QTVATHYHY

SEQ ID NO: 100 - protein, 9 aa, Artificial Sequence
  Synthetic
WHAKHLLSH

SEQ ID NO: 101 - protein, 9 aa, Artificial Sequence
  Synthetic
SGIKKADSV

SEQ ID NO: 102 - protein, 9 aa, Artificial Sequence
  Synthetic
KANLINVKS

SEQ ID NO: 103 - protein, 9 aa, Artificial Sequence
  Synthetic
WKSHVEVRS

SEQ ID NO: 104 - protein, 9 aa, Artificial Sequence
  Synthetic
WVTQKYIIQ

SEQ ID NO: 105 - protein, 9 aa, Artificial Sequence
  Synthetic
WEHAKDWPT

SEQ ID NO: 106 - protein, 9 aa, Artificial Sequence
  Synthetic
FHSKVLDKA

SEQ ID NO: 107 - protein, 9 aa, Artificial Sequence
  Synthetic
YGAQKQAVW

SEQ ID NO: 108 - protein, 9 aa, Artificial Sequence
  Synthetic
FSASHFTSQ

SEQ ID NO: 109 - protein, 9 aa, Artificial Sequence
  Synthetic
  misc_feature (1)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
XXAXKXDXQ

SEQ ID NO: 110 - protein, 112 aa, Artificial Sequence
  Synthetic
MIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 111 - protein, 112 aa, Artificial Sequence
  Synthetic
MIPRGLSEAKPATPEIQEIVDKVKPQLEEKTNETYGKLEAVQYKTQVLAGYWAAKWPGSTNYYIKVRAGDNKYMHLKVFNGPFPNTSYDLQADRVLTGYQVDKNKDDELTGF

SEQ ID NO: 112 - protein, 112 aa, Artificial Sequence
  Synthetic
MIPRGLSEAKPATPEIQEIVDKVKPQLEEKTNETYGKLEAVQYKTQVLANWYQQRWPGSTNYYIKVRAGDNKYMHLKVFNGPWHNYGESSGADRVLTGYQVDKNKDDELTGF

SEQ ID NO: 113 - protein, 112 aa, Artificial Sequence
  Synthetic
MIPRGLSEAKPATPEIQEIVDKVKPQLEEKTNETYGKLEAVQYKTQVLAVWGPEYQHQSTNYYIKVRAGDNKYMHLKVFNGPNAGWPLVPEADRVLTGYQVDKNKDDELTGF

SEQ ID NO: 114 - protein, 112 aa, Artificial Sequence
  Synthetic
MIPRGLSEAKPATPEIQEIVDKVKPQLEEKTNETYGKLEAVQYKTQVLAGHYARYWPGSTNYYIKVRAGDNKYMHLKVFNGPWAQKSKVHQADRVLTGYQVDKNKDDELTGF

SEQ ID NO: 115 - protein, 112 aa, Artificial Sequence
  Synthetic
MIPRGLSEAKPATPEIQEIVDKVKPQLEEKTNETYGKLEAVQYKTQVLAGFWASKWPGSTNYYIKVRAGDNKYMHLKVFNGPFTAVSKKDAADRVLTGYQVDKNKDDELTGF

SEQ ID NO: 116 - protein, 112 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 117 - protein, 254 aa, Artificial Sequence
  Synthetic
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 118 - protein, 255 aa, Artificial Sequence
  Synthetic
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 119 - protein, 254 aa, Artificial Sequence
  Synthetic
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 120 - protein, 255 aa, Artificial Sequence
  Synthetic
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 121 - protein, 254 aa, Artificial Sequence
  Synthetic
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 122 - protein, 255 aa, Artificial Sequence
  Synthetic
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 123 - protein, 398 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 124 - protein, 398 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 125 - protein, 398 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 126 - protein, 399 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFC

SEQ ID NO: 127 - protein, 398 aa, Artificial Sequence
  Synthetic
MIPRGLSEAKPATPEIQEIVDKVKPQLEEKTNETYGKLEAVQYKTQVLAFALPEFEYMSTNYYIKVRAGDNKYMHLKVFNGPPMIRRKNEVADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAFALPEFEYMSTNYYIKVRAGDNKYMHLKVFNGPPMIRRKNEVADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 128 - protein, 398 aa, Artificial Sequence
  Synthetic
MIPRGLSEAKPATPEIQEIVDKVKPQLEEKTNETYGKLEAVQYKTQVLAGGGGGGGGGSTNYYIKVRAGDNKYMHLKVFNGPGGGGGGGGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAGGGGGGGGGSTNYYIKVRAGDNKYMHLKVFNGPGGGGGGGGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 129 - protein, 255 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 130 - protein, 256 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFC

SEQ ID NO: 131 - protein, 255 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 132 - protein, 254 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 133 - protein, 149 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGCAENLYFQGGAAGHHHHHH

SEQ ID NO: 134 - protein, 9 aa, Artificial Sequence
  Synthetic
WTQPKNEHH

SEQ ID NO: 135 - protein, 9 aa, Artificial Sequence
  Synthetic
HLKHTDAQP

SEQ ID NO: 136 - protein, 4 aa, Artificial Sequence
  Synthetic
PPGP

SEQ ID NO: 137 - protein, 7 aa, Artificial Sequence
  Synthetic
  misc_feature (1)..(1): Xaa may be Asp or Glu
  misc_feature (2)..(2): Xaa may be Arg or Lys
  misc_feature (4)..(4): Xaa may be Asp or Glu
  misc_feature (5)..(5): Xaa may be Thr or Ser
XXGXXGP

SEQ ID NO: 138 - protein, 7 aa, Artificial Sequence
  Synthetic
DRGETGP

SEQ ID NO: 139 - protein, 4 aa, Artificial Sequence
  Synthetic
  misc_feature (4)..(4): Xaa can be any naturally occurring amino acid
GPAX

SEQ ID NO: 140 - protein, 10 aa, Artificial Sequence
  Synthetic
  misc_feature (2)..(2): Xaa may be Trp or Phe
  misc_feature (3)..(3): Xaa may be Tyr or Phe
NXXQQRRWPG

SEQ ID NO: 141 - protein, 9 aa, Artificial Sequence
  Synthetic
  misc_feature (1)..(1): Xaa may be Asp or Gly
  misc_feature (2)..(2): Xaa may be Trp, Tyr, or Phe
  misc_feature (4)..(4): Xaa may be Gln or Ala
  misc_feature (5)..(5): Xaa may be Ala or Ser
  misc_feature (9)..(9): Xaa may be His or Gly
XXWXXKWPX

SEQ ID NO: 142 - protein, 9 aa, Artificial Sequence
  Synthetic
  misc_feature (2)..(2): Xaa may be Tyr, Phe, or His
  misc_feature (3)..(3): Xaa may be Trp or Tyr
  misc_feature (5)..(5): Xaa may be Ala, Ser, or Arg
  misc_feature (6)..(6): Xaa may be Lys or Tyr
GXXAXXWPG

SEQ ID NO: 143 - protein, 14 aa, Artificial Sequence
  Synthetic
  misc_feature (1)..(1): Xaa may be Pyroglutamic acid
XQRLGNQWAVGHLM

SEQ ID NO: 144 - protein, 10 aa, Artificial Sequence
  Synthetic
TAASGVRSMH

SEQ ID NO: 145 - protein, 10 aa, Artificial Sequence
  Synthetic
LTLRWVGLMS

SEQ ID NO: 146 - protein, 9 aa, Artificial Sequence
  Synthetic
  misc_feature (1)..(1): Xaa may be Pyroglutamic acid
XHWSYSLRP

SEQ ID NO: 147 - protein, 10 aa, Artificial Sequence
  Synthetic
  misc_feature (1)..(1): Xaa may be Pyroglutamic acid
XHWSYGLRPG

SEQ ID NO: 148 - protein, 9 aa, Artificial Sequence
  Synthetic
  misc_feature (1)..(1): Xaa may be Pyroglutamic acid
XHWSYSLRP

SEQ ID NO: 149 - protein, 9 aa, Artificial Sequence
  Synthetic
  misc_feature (1)..(1): Xaa may be Pyroglutamic acid
XHWSYHLRP

SEQ ID NO: 150 - protein, 9 aa, Artificial Sequence
  Synthetic
  misc_feature (1)..(1): Xaa may be Pyroglutamic acid
XHWSYLLRP

SEQ ID NO: 151 - protein, 9 aa, Artificial Sequence
  Synthetic
  misc_feature (1)..(1): Xaa may be Pyroglutamic acid
XHWSYLRPG

SEQ ID NO: 152 - protein, 10 aa, Artificial Sequence
  Synthetic
  misc_feature (1)..(1): Xaa may be Pyroglutamic acid
XHWSYWLRPG

SEQ ID NO: 153 - protein, 9 aa, Artificial Sequence
  Synthetic
FASYNLKPA

SEQ ID NO: 154 - protein, 8 aa, Artificial Sequence
  Synthetic
FASWKRPA

SEQ ID NO: 155 - protein, 9 aa, Artificial Sequence
  Synthetic
FASFFLKPA

SEQ ID NO: 156 - protein, 9 aa, Artificial Sequence
  Synthetic
FASYRLRPA

SEQ ID NO: 157 - protein, 7 aa, Artificial Sequence
  Synthetic
GRGDSPK

SEQ ID NO: 158 - protein, 6 aa, Artificial Sequence
  Synthetic
NRGDFV

SEQ ID NO: 159 - protein, 5 aa, Artificial Sequence
  Synthetic
  misc_feature (2)..(2): Xaa may be Sarcosine
RXDFV

SEQ ID NO: 160 - protein, 5 aa, Artificial Sequence
  Synthetic
RGDFV

SEQ ID NO: 161 - protein, 5 aa, Artificial Sequence
  Synthetic
RGDFK

SEQ ID NO: 162 - protein, 5 aa, Artificial Sequence
  Synthetic
RGDFC

SEQ ID NO: 163 - protein, 5 aa, Artificial Sequence
  Synthetic
RADFC

SEQ ID NO: 164 - protein, 5 aa, Artificial Sequence
  Synthetic
RADFK

SEQ ID NO: 165 - protein, 5 aa, Artificial Sequence
  Synthetic
RGDFE

SEQ ID NO: 166 - protein, 5 aa, Artificial Sequence
  Synthetic
RADFE

SEQ ID NO: 167 - protein, 5 aa, Artificial Sequence
  Synthetic
RGDSK

SEQ ID NO: 168 - protein, 5 aa, Artificial Sequence
  Synthetic
RADSK

SEQ ID NO: 169 - protein, 4 aa, Artificial Sequence
  Synthetic
RGDS

SEQ ID NO: 170 - protein, 5 aa, Artificial Sequence
  Synthetic
RGDYC

SEQ ID NO: 171 - protein, 5 aa, Artificial Sequence
  Synthetic
RADYC

SEQ ID NO: 172 - protein, 5 aa, Artificial Sequence
  Synthetic
RGDYE

SEQ ID NO: 173 - protein, 5 aa, Artificial Sequence
  Synthetic
RGDYK

SEQ ID NO: 174 - protein, 5 aa, Artificial Sequence
  Synthetic
RADYK

SEQ ID NO: 175 - protein, 12 aa, Artificial Sequence
  Synthetic
HERGDYKRGDYK

SEQ ID NO: 176 - protein, 6 aa, Artificial Sequence
  Synthetic
RGNDFV

SEQ ID NO: 177 - protein, 6 aa, Artificial Sequence
  Synthetic
RGDNFV

SEQ ID NO: 178 - protein, 6 aa, Artificial Sequence
  Synthetic
RGDFNV

SEQ ID NO: 179 - protein, 8 aa, Artificial Sequence
  Synthetic
TGRGPSWV

SEQ ID NO: 180 - protein, 8 aa, Artificial Sequence
  Synthetic
SARGPSRW

SEQ ID NO: 181 - protein, 8 aa, Artificial Sequence
  Synthetic
TARGPSFK

SEQ ID NO: 182 - protein, 8 aa, Artificial Sequence
  Synthetic
LSGRSDNH

SEQ ID NO: 183 - protein, 8 aa, Artificial Sequence
  Synthetic
GGWHTGRN

SEQ ID NO: 184 - protein, 8 aa, Artificial Sequence
  Synthetic
HTGRSGAL

SEQ ID NO: 185 - protein, 8 aa, Artificial Sequence
  Synthetic
PLTGRSGG

SEQ ID NO: 186 - protein, 8 aa, Artificial Sequence
  Synthetic
AARGPAIH

SEQ ID NO: 187 - protein, 8 aa, Artificial Sequence
  Synthetic
RGPAFNPM

SEQ ID NO: 188 - protein, 8 aa, Artificial Sequence
  Synthetic
SSRGPAYL

SEQ ID NO: 189 - protein, 8 aa, Artificial Sequence
  Synthetic
RGPATPIM

SEQ ID NO: 190 - protein, 4 aa, Artificial Sequence
  Synthetic
RGPA

SEQ ID NO: 191 - protein, 10 aa, Artificial Sequence
  Synthetic
GGQPSGMWGW

SEQ ID NO: 192 - protein, 10 aa, Artificial Sequence
  Synthetic
FPRPLGITGL

SEQ ID NO: 193 - protein, 10 aa, Artificial Sequence
  Synthetic
VHMPLGFLGP

SEQ ID NO: 194 - protein, 8 aa, Artificial Sequence
  Synthetic
SPLTGRSG

SEQ ID NO: 195 - protein, 8 aa, Artificial Sequence
  Synthetic
SAGFSLPA

SEQ ID NO: 196 - protein, 9 aa, Artificial Sequence
  Synthetic
LAPLGLQRR

SEQ ID NO: 197 - protein, 8 aa, Artificial Sequence
  Synthetic
SGGPLGVR

SEQ ID NO: 198 - protein, 4 aa, Artificial Sequence
  Synthetic
PLGL

SEQ ID NO: 199 - protein, 7 aa, Artificial Sequence
  Synthetic
GPRSFGL

SEQ ID NO: 200 - protein, 6 aa, Artificial Sequence
  Synthetic
GPRSFG

SEQ ID NO: 201 - protein, 8 aa, Artificial Sequence
  Synthetic
ISSGLLSS

SEQ ID NO: 202 - protein, 8 aa, Artificial Sequence
  Synthetic
QNQALRMA

SEQ ID NO: 203 - protein, 8 aa, Artificial Sequence
  Synthetic
AQNLLGMV

SEQ ID NO: 204 - protein, 8 aa, Artificial Sequence
  Synthetic
STFPFGMF

SEQ ID NO: 205 - protein, 8 aa, Artificial Sequence
  Synthetic
PVGYTSSL

SEQ ID NO: 206 - protein, 8 aa, Artificial Sequence
  Synthetic
DWLYWPGI

SEQ ID NO: 207 - protein, 8 aa, Artificial Sequence
  Synthetic
MIAPVAYR

SEQ ID NO: 208 - protein, 8 aa, Artificial Sequence
  Synthetic
RPSPMWAY

SEQ ID NO: 209 - protein, 8 aa, Artificial Sequence
  Synthetic
WATPRPMR

SEQ ID NO: 210 - protein, 8 aa, Artificial Sequence
  Synthetic
FRLLDWQW

SEQ ID NO: 211 - protein, 8 aa, Artificial Sequence
  Synthetic
LKAAPRWA

SEQ ID NO: 212 - protein, 8 aa, Artificial Sequence
  Synthetic
GPSHLVLT

SEQ ID NO: 213 - protein, 8 aa, Artificial Sequence
  Synthetic
LPGGLSPW

SEQ ID NO: 214 - protein, 8 aa, Artificial Sequence
  Synthetic
MGLFSEAG

SEQ ID NO: 215 - protein, 8 aa, Artificial Sequence
  Synthetic
SPLPLRVP

SEQ ID NO: 216 - protein, 8 aa, Artificial Sequence
  Synthetic
RMHLRSLG

SEQ ID NO: 217 - protein, 8 aa, Artificial Sequence
  Synthetic
LAAPLGLL

SEQ ID NO: 218 - protein, 8 aa, Artificial Sequence
  Synthetic
AVGLLAPP

SEQ ID NO: 219 - protein, 8 aa, Artificial Sequence
  Synthetic
LLAPSHRA

SEQ ID NO: 220 - protein, 8 aa, Artificial Sequence
  Synthetic
PAGLWLDP

SEQ ID NO: 221 - protein, 7 aa, Artificial Sequence
  Synthetic
ISSGLSS

(19) United States
(12) Patent Application Publication
(10) Pub. No.: US 2023/0381330 A1
(43) Pub. Date: Nov. 30, 2023

(54) ENZYME-ACTIVATED SERUM EXTENDED HALF-LIFE THERAPEUTIC CONJUGATES

(71) Applicants: Avacta Life Sciences Limited, Wetherby (GB); Trustees of Tufts College, Medford, MA (US)

(72) Inventors: Amrik Basran, Cambridge (GB); Matthew P. Vincent, Amesbury, MA (US); Emma Jenkins, Stansted (GB); Estelle Adam, Hitchin (GB); William Bachovchin, Melrose, MA (US)

(73) Assignees: Avacta Life Sciences Limited, Wetherby (GB); Trustees of Tufts College, Medford, MA (US)

(21) Appl. No.: 18/034,221

(22) PCT Filed: Oct. 29, 2021
(86) PCT No.: PCT/US2021/057291

§ 371 Date: Apr. 27, 2023

Related U.S. Application Data
(60) Provisional application No. 63/197,935, filed on Jun. 7, 2021.
(60) Provisional application No. 63/108,046, filed on Oct. 30, 2020.

(51) Int. Cl. A61K 47/65; A61K 47/64; A61K 47/68; A61P 35/00; A61K 47/55

(52) U.S. Cl. CPC A61K 47/65; A61K 47/643; A61K 47/68; A61P 35/00; A61K 47/551

(57) ABSTRACT

Disclosed herein are therapeutic conjugates having an extended circulating serum half-life. The therapeutic conjugates comprise a drug moiety linked to a half-life extension moiety via an enzyme-cleavable linker. Methods of using the therapeutic conjugates are also provided.

### RELATED APPLICATIONS

This application claims the benefit under 35 U.S.C. § 119(e) of U.S. 63/108,046, filed Oct. 30, 2020 and U.S. 63/197,935, filed Jun. 7, 2021, which are incorporated by reference herein in their entireties.

### BACKGROUND

Various therapeutic drugs lack practical efficacy due to immune-related toxicities and other dose-limiting factors, for example, drugs that induce innate immune responses; however, these drugs are frequently too toxic for systemic use. This is because systemic innate immune activation greatly limits the dose that may be administered. Therefore, the maximum tolerated doses do not reach a therapeutic level in many subjects.

### SUMMARY

Disclosed are serum half-life extended drug conjugates of therapeutic or imaging agents, which drug conjugates have a linker including a substrate that can be selectively cleaved by an enzyme, for example, present extracellularly in a cancerous tissue, to release the drug moiety at the site of cleavage. Upon cleavage by the enzyme of the substrate, the therapeutic conjugate releases the drug moiety in its active form or in a form that is readily metabolized to its active form. Pharmaceutical compositions comprising the drug conjugates, as well as methods of using the moiety conjugates to treat cancer are also disclosed.

In some aspects, are therapeutic conjugates designed to release a therapeutically effective amount of a drug (or other drug moiety) in a tumor microenvironment of a subject without inducing acute toxicity in the subject. Surprisingly, the conjugates of the present disclosure are capable of local delivery of a therapeutically effective amount of a drug, even in the absence of a cell-binding (e.g., targeting) moiety. Instead of a targeting moiety or other cell-specific homing mechanism, the conjugates of the present disclosure include a half-life extension moiety and a cleavable linker that enables local accumulation and release of the drug (or other drug moiety) specifically in a tumor microenvironment. The half-life extension moiety enables local accumulation of the drug, while the specificity is achieved using a cleavable linker that is cleaved specifically by an enzyme present at high levels in tumor microenvironments. Thus, specific cleavage of the linker by the enzyme results in local release of a therapeutically effective amount of the drug in a tumor microenvironment, without the toxic side effects typically associated with systemic delivery of the drug.

Some aspects of the present disclosure provide a therapeutic conjugate comprising a drug moiety linked through an enzyme-cleavable linker to a half-life extension moiety, wherein the circulating serum half-life of the therapeutic conjugate in vivo is at least 48 hours, and the conjugate does not comprise a cell-binding moiety that binds to a cell surface protein of a cell with a K_{d} of 1×10⁻⁶ M or less.

Other aspects of the present disclosure provide a therapeutic conjugate comprising a drug moiety linked through an enzyme-cleavable linker to a half-life extension moiety, wherein the circulating serum half-life of the therapeutic conjugate in vivo is at least 48 hours, and the conjugate does not comprise a cell-binding moiety that binds to a cell surface protein of a cell in a tumor microenvironment.

Yet other aspects of the present disclosure provide a therapeutic conjugate comprising a drug moiety linked through an enzyme-cleavable linker to a half-life extension moiety, wherein the circulating serum half-life of the therapeutic conjugate in vivo is extended by at least 2-fold relative to circulating serum half-life of the drug moiety not linked to the half-life extension moiety, and the conjugate does not comprise a cell-binding moiety that binds to a cell surface protein of a cell with a K_{d} of 1×10⁻⁶ M or less.

Still other aspects of the present disclosure provide a therapeutic conjugate comprising a drug moiety linked through an enzyme-cleavable linker to a half-life extension moiety, wherein the circulating serum half-life of the therapeutic conjugate in vivo is extended by at least 2-fold relative to circulating serum half-life of the drug moiety not linked to the half-life extension moiety, and the conjugate does not comprise a cell-binding moiety that binds to a cell surface protein of a cell in a tumor microenvironment.

The drug moiety not linked to the half-life extension moiety is referred to herein as a “free” drug moiety (i.e., the parent molecule of the drug moiety resulting from cleavage of the conjugate by the enzyme).

In some embodiments, the half-life extension moiety comprises a serum protein. For example, the serum protein may be selected from fibronectin, transferrin, and human serum albumin (HSA).

In some embodiments, the half-life extension moiety comprises a molecule that binds to a serum protein. For example, the molecule that binds to a serum protein may be fibronectin, transferrin, or HSA.

In some embodiments, the molecule that binds to a serum protein the molecule that binds to a serum protein is an antibody. The antibody, for example, may be an Fab, F(ab)2, F(ab′), F(ab′)2, F(ab′)3, Fd, Fv, disulfide linked Fv, dAb or sdAb (or NANOBODY®), CDR, scFv, (scFv)2, di-scFv, bi-scFv, tascFv (tandem scFv), AVIBODY® (e.g., diabody, triabody, and tetrabody), T-cell engager (BiTE®), Fc, scFv-Fc, Fcab, mAb2, small modular immunopharmaceutical (SMIP), Genmab/unibody or duobody, V-NAR domain, IgNAR, minibody, IgGACH2, DVD-Ig, probody, intrabody, or a multispecificity antibody.

In some embodiments, the molecule that binds to a serum protein is a non-antibody molecule. The non-antibody molecule, for example, may be an affibody, an AFFIMER® polypeptide, an affilin, an anticalin, an atrimer, an avimer, a DARPin, an FN3 scaffold (e.g., Adnectins, Centyrins), a fynomer, a Kunitz domain, a nanofitin, a pronectins, a tribody, bicyclic peptides, or a Cys-knot. Other non-antibody molecules that bind to a serum protein are encompassed by the present disclosure.

In some embodiments, the half-life extension moiety comprises an HSA-binding recombinantly engineered variant of stefin polypeptide (i.e., AFFIMER® polypeptide).

In some embodiments, the recombinantly engineered variant of stefin polypeptide (i.e., AFFIMER® polypeptide) comprises an amino acid sequence that has at least 70%, at least 80%, at least 90%, or 100% identity to an amino acid sequence of any one of SEQ ID NOS: 110-132.

In some embodiments, the half-life extension moiety comprises an antibody Fc domain, optionally from IgA, IgD, IgE, IgG, or IgM or a subclass thereof.

In some embodiments, the half-life extension moiety comprises a biocompatible polymer, optionally selected from the group consisting of a poly(ethylene glycol) (PEG), a hydroxyethyl starch, an XTEN™ polymer, and a proline-alanine-serine polymer.

In some embodiments, the therapeutic conjugate is represented by one of the formula: X-L¹-SRS-L²-DM, wherein X is the half-life extension moiety, L¹ is a spacer or bond, SRS is a substrate recognition sequence cleavable by an enzyme, e.g., present in a tumor microenvironment, L² is a self-immolative linker (e.g., which is metabolized or otherwise eliminated after FAPα cleavage to release the free therapeutic moiety) or bond, and DM is the drug moiety.

In some embodiments, the therapeutic conjugate is represented by one of the formula: X-(L¹-SRS-L²-DM)ₙ; X-L¹-(SRS-L²-DM)ₙ; (X)ₘ-(L¹-SRS-L²-DM)ₙ; or (X)ₘ-L¹-(SRS-L²-DM)ₙ, wherein X is the half-life extension moiety, L₁ is a spacer or bond, SRS is a substrate recognition sequence cleavable by an enzyme, e.g., in a tumor microenvironment, L² is a self-immolative linker or bond, DM is the drug moiety, m is an integer from 1 to 6, and n is an integer from 1 to 500, optionally 1 to 100, 1 to 10, or 1 to 5.

In some embodiments, the therapeutic conjugate comprises a moiety capable of chemically conjugating in vivo to albumin or other proteins in circulation in the serum of the patient. In some embodiments, the therapeutic conjugate is represented by one of the formula: Y-L¹-SRS-L²-DM; Y-(L¹-SRS-L²-DM)ₙ; or Y-L¹-(SRS-L²-DM)ₙ, wherein L¹, SRS, L² and TM are as defined above, and Y is a reactive group capable of chemically cross-linking to a protein in vivo.

In some embodiments, Y is a reactive group capable of chemically cross-linking to free amines (such as the sidechain of lysines) present in a protein. A non-limiting example includes a linker including NHS (N-hydroxysuccinimide), such as shown in FIG. 6A.

In some embodiments, Y is a reactive group capable of chemically cross-linking to free thiol groups (such as the sidechain of cysteine) present in a protein. A non-limiting example includes a linker including maleimide, such as shown in FIG. 6B.

In some embodiments, the enzyme-cleavable linker is present extracellularly in a diseased tissue, optionally a cancerous tissue.

In some embodiments, the enzyme-cleavable linker is an oligopeptide.

In some embodiments, the oligopeptide comprises a C-terminal proline covalently linked to the drug moiety, optionally via a bond or a self-immolative linker, and/or an N-terminal blocking group.

In some embodiments, the bond can be cleaved by the proteolytic activity of the enzyme, optionally wherein the bond is an amide bond.

In some embodiments, the self-immolative linker comprises a heterocyclic self-immolative moiety, optionally His-Ala, p-aminobenzyloxycarbonyl (PABC) or and 2,4-bis(hydroxymethyl)aniline.

In some embodiments, the substrate recognition sequence is cleaved by a protease.

In some embodiments, the protease is a serine protease, metal protease, or cysteine protease.

In some embodiments, the protease is present extracellularly in the cancerous state tissue in a subject at levels at least 2, 3, 4, 5, 6, 7, 8, 9, 10, 15, 20, 30, 40, 50, 60, 70, 80, 90, or 100 times greater than the healthy state of the tissue in the subject.

In some embodiments, the protease is present extracellularly in the cancerous state of the tissue in a subject at levels at least 2, 3, 4, 5, 6, 7, 8, 9, 10, 15, 20, 30, 40, 50, 60, 70, 80, 90 or 100 times greater than other tissue of the subject.

In some embodiments, the protease is a matrix metalloproteinase selected from membrane bound matrix metalloproteinases (such as MMP14-17 and MMP24-25) and secreted matrix metalloproteinases (such as MMP1-13 and MMP18-23 and MMP26-28).

In some embodiments, the protease is selected from the group consisting of MMP1, MMP2, MMP3, MMP4, MMP9, MMP11, MMP13, MMP14, MMP17, and MMP19.

In some embodiments, the protease is an A Disintegrin and Metalloproteinase (ADAM), or an A Disintegrin or Metalloproteinase with Thrombospondin Motifs (ADAMTS).

In some embodiments, the protease is selected from the group consisting of a legumain, a matriptase (MT-SP1), a neutrophil elastase, a TMPRSS, a thrombin, a u-type plasminogen activator (uPA, also referred to as urokinase), PSMA, and CD 10 (CALLA).

In some embodiments, the enzyme-cleavable linker has a kcat/Km for cleavage by FAPα at least 10-fold, at least 100-fold, 1000-fold, 5000-fold, or 10,000-fold greater than a kcat/Km for cleavage by prolyl endopeptidase (EC 3.4.21.26; PREP).

In some embodiments, the enzyme-cleavable linker is a FAPα-cleavable linker. In some embodiments, the FAPα-cleavable linker comprises a sequence selected from a D-Ala-Pro, PPGP (SEQ ID NO: 136), (D/E)-(R/K)-G-(E/D)-(T/S)-G-P (SEQ ID NO: 137), DRGETGP (SEQ ID NO: 138), and GPAX (SEQ ID NO: 139), optionally a D-Ala-Pro sequence or a D-Ser sequence.

In some embodiments, the substrate recognition sequence (SRS) of the FAPα-cleavable linker is represented by

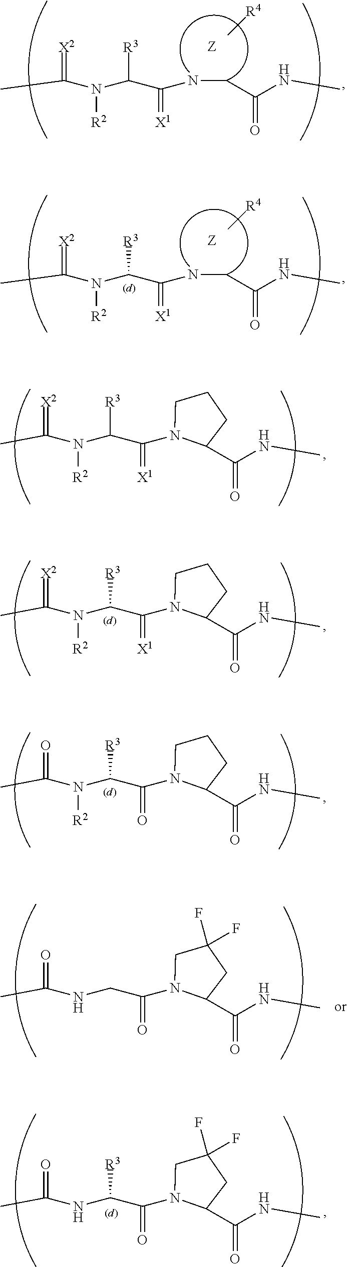

wherein R² is hydrogen or (C₁-C₆) alkyl or hydrogen, R³ is hydrogen or a branched or straight chain lower alkyl, e.g., a lower alkyl such as methyl (if (d) is an amino acid side chain, then R³ is not hydrogen), R⁴ is a branched or straight chain lower alkoxy, such as hydroxymethyl (e.g., for serine) or 1-hydroxyethyl (e.g., for threonine) (in one embodiment, R⁴ is hydroxymethyl), X¹ is O or S, and/or X² is O or S.

In some embodiments, the substrate recognition sequence of the FAPα-cleavable linker comprises a third amino position, optionally N-terminal to (d)-Ala (or other (d)-amino acid in that position and formed by R3), and optionally wherein the amino acid at the third amino acid position is serine or threonine.

In some embodiments, the FAPα-cleavable linker has a kcat/Km for cleavage by FAPα at least 10-fold, at least 100-fold, 1000-fold, 5000-fold, or 10,000-fold greater than a kcat/Km for cleavage by prolyl endopeptidase (EC 3.4.21.26; PREP).

In some embodiments, the drug moiety induces an innate immune (versus adaptive immune) response in vivo. In some embodiments, the drug moiety is acutely toxic. Acute toxicity describes the adverse effects of a drug, for example, that result either from a single exposure or from multiple exposures in a short period of time (e.g., less than 24 hours). The adverse effects of acute toxicity typically occur within 14 days of the administration of the drug.

In some embodiments, the drug moiety is a cytotoxic agent, i.e., which when released from the therapeutic conjugate causes cell death of the target cells at the concentration at which the therapeutic conjugate is administered.

In some embodiments, the drug moiety is a cytostatic agent, i.e., which when released from the therapeutic conjugate causes mitotic arrest or quiescence of the target cells at the concentration at which the therapeutic conjugate is administered.

In some embodiments, the drug moiety is an epigenetic agent, i.e., which when released from the therapeutic conjugate causes epigenetic alteration of the target cells at the concentration at which the therapeutic conjugate is administered, which may, for example, result in differentiation (or dedifferentiation) of the cell to another cellular phenotype.

In some embodiments, the drug moiety is selected from TLR agonists, RIG-I agonists, iDASH inhibitors, and STING agonists.

In some embodiments, the drug moiety is a TLR agonist, such as a selected from the group consisting of a TLR1/2 agonist, a TLR2 agonist, a TLR3 agonist, a TLR4 agonist, a TLR5 agonist, a TLR6/2 agonist, a TLR7 agonist, a TLR7/8 agonist, a TLR7/9 agonist, a TLR8 agonist, a TLR9 agonist, and a TLR11 agonist, preferably selected from the group consisting of a TLR3 agonist, a TLR7 agonist, a TLR7/8 agonist, and a TLR9 agonist.

In some embodiments, the drug moiety is an iDASH inhibitor that inhibits the enzymatic activity of DPP8 and DPP9 and induces macrophage pyroptosis.

In some embodiments, the drug moiety is Val-boroPro (Talabostat).

In some embodiments, the drug moiety includes a ligand for a receptor, such as ligand that when released from the therapeutic conjugate is able to bind to an extracellular ligand binding domain of a cell surface receptor. Exemplary receptor ligands include somatostatin, cholecystokinin-2 (CCK2), folate, bombesin, gastrin-releasing peptide, neurotensin, substance P, glucagon-like peptide 1, neuropeptide Y and analogs of those ligands. The receptor ligand can itself have pharmacological activity or can be used to deliver a conjugated drug moiety, toxin or radioisotope.

In some embodiments, the drug moiety DM is represented by the general formula

—RBM [symbol] Z)ₚ

wherein RBM is a receptor binding moiety, Z is cytotoxic, cytostatic or epigenetic moiety or a radioisotope containing moiety, and p is 0 (Z is absent) or an integer from 1 to 8. In some embodiments, p is 1.

In some embodiments, the drug moiety DM is represented by the general formula

—RBM-L³-Z

wherein RBM is a receptor binding moiety, Z is cytotoxic, cytostatic or epigenetic moiety or a radioisotope containing moiety, and L³ is a bond or a cleavable or non-cleavable linker. For instance, L³ can be a linker that is acid labile or enzyme sensitive (such as includes a cathepsin cleavage site) such that Z is released intracellularly on internalization of the moiety —RBM-L³-Z through cell binding dependent on the receptor binding moiety RBM.

In some embodiments, the drug moiety has a circulating serum half-life that it is at least 5, 10, 25, 50, 100 or even 1000 times longer than the circulating serum half-life of the free drug moiety. The circulating serum half-life of a drug moiety is a pharmacokinetic parameter that is defined as the time it takes for the concentration of the drug moiety in the serum to be reduced by 50%.

In some embodiments, the drug moiety has a circulating serum half-life that it is at least 10, 25, 50, 100, 200, or 300 hours.

In some embodiments, for a period of at least 10, 24, 48, 72, 96, or 120 hours the therapeutic conjugate produces a concentration of free drug moiety in target tissue expressing the enzyme that is at least 2, 5, 10, 20, 50, 75 or 100 times the concentration of free drug moiety in systemic circulation over the same period of time. For instance, such differences in free drug moiety concentrations can occur in subjects between tumors expressing the enzyme and serum.

In some embodiments, when administered to a subject having a tumor expressing the enzyme, the therapeutic conjugate produces an intratumoral concentration of free drug moiety that is at or above the EC₅₀ for the antitumor activity of the free drug moiety for a period at least 10, 24, 48, 72, 96, or 120 hours.

In some embodiments, when administered to a subject having a tumor expressing the enzyme, the therapeutic conjugate has a therapeutic index for antitumor activity of at least 2, 5, 10, 25, 50, 100, or 500. The therapeutic index (TI) is a quantitative measurement of the relative safety of a drug. It is a comparison of the amount of a therapeutic agent that causes the therapeutic effect to the amount that causes toxicity (TI also referred to as a therapeutic ratio).

In some embodiments, when administered to a subject having a tumor expressing the enzyme, the therapeutic conjugate has a therapeutic index for antitumor activity that is at least 2, 10, 50, 100, 250, 500, 1000, 5000, or 10,000 times greater than the therapeutic index of the free drug moiety.

In some embodiments, a greater percentage of free drug moiety is localized in a target tissue expressing the enzyme, relative to free drug moiety, when compared on an equivalent dose basis, optionally wherein the ratio of free drug moiety localized in the target tissue relative to other tissue (such as blood, liver or heart) is at least 2, 5, 10, 100, or 1000 times greater for an equivalent dose of the therapeutic conjugate relative to the free drug moiety.

In some embodiments, the maximum tolerated dose of the therapeutic conjugate is at least 2, 5, 10, 100, or 1000 times greater than the maximum tolerated dose of the free drug moiety. The maximum tolerated dose is the highest dose of a drug moiety (e.g., drug) that does not cause unacceptable side effects or overt toxicity in a specific period of time. The maximum tolerated dose may be determined in clinical trials by testing increasing doses on different groups of people until the highest dose with acceptable side effects is found.

In some embodiments, the cell permeability of the therapeutic conjugate is at least 50%, 60%, 70%, 80%, 90%, 95%, 98%, 99%, or 99.9% less than the cell permeability of free drug moiety.

In some embodiments, the circulating half-life of the therapeutic conjugate is at least 25%, 50%, 75%, 100%, 150%, 200%, 500%, 750%, or 1000% longer than the circulating half-life of free drug moiety.

In some embodiments, the therapeutic conjugate has less than 50%, 60%, 70%, 80%, 90%, 95%, 98%, 99%, 99.9%, or 99.99% of the cytotoxic or cytolytic activity against tumor cells relative to free drug moiety.

Also provided herein, in some aspects, is a composition comprising the therapeutic conjugate of any one of the preceding paragraphs and a pharmaceutically acceptable excipient.

Further provided herein, in some aspects, is a method comprising administering to a subject the therapeutic conjugate or composition of any one of the preceding paragraphs, wherein the subject has a diseased tissue, optionally a cancer.

In some embodiments, the therapeutic conjugate or composition is administered in an amount effective to increase lactate dehydrogenase (LDH) release in a tumor microenvironment the subject by at least 0.5-fold or at least 1-fold relative to an untreated control subject. In some embodiments, the therapeutic conjugate or composition is administered in an amount effective to increase LDH release in a tumor microenvironment the subject by about 0.5-fold, about 0.6-fold, about 0.7-fold, about 0.8-fold, about 0.9-fold, about 1-fold, about 1.5-fold, or about 2-fold. In some embodiments, the therapeutic conjugate or composition is administered in an amount effective to increase LDH release in a tumor microenvironment the subject by about 0.5-fold to about 1.5-fold, about 0.5-fold to about 2-fold, or about 1-fold to about 2-fold.

In some embodiments, the volume of the diseased tissue, optionally a tumor, is reduced by at least 50%, at least 60%, or at least 70% at about 2-3 weeks following administration of the therapeutic conjugate or composition. In some embodiments, the volume of the diseased tissue, optionally a tumor, is reduced by about 50%, about 55%, about 60%, about 65%, about 70%, about 75%, or about 80%. In some embodiments, the volume of the diseased tissue, optionally a tumor, is reduced by about 50% to about 60%, or about 50% to about 70%, or about 50% to about 80%.

Some aspects provide a therapeutic conjugate or composition of any one of the preceding paragraphs for use in a method for treating a diseased tissue, optionally a cancer.

Other aspects provide a use of the therapeutic conjugate of any one of the preceding paragraphs in the manufacture of a medicament for the treatment of a diseased tissue, optionally a cancer.

Some aspects of the present disclosure provide a therapeutic conjugate comprising a drug moiety linked through an enzyme-cleavable linker to a half-life extension moiety, wherein the chemodrug moiety induces an innate immune response in vivo and/or is acutely toxic, the circulating serum half-life of the therapeutic conjugate in vivo is at least 48 hours, and the therapeutic conjugate does not comprise a cell-binding moiety that binds to a cell surface protein of a cell with a K_{d} of 1×10⁻⁸ M or less. In some embodiments, the chemodrug moiety is selected from TLR agonists, RIG-I agonists, iDASH inhibitors, and STING agonists. In some embodiments, the chemodrug moiety is Val-boroPro (Talabostat). In some embodiments, the half-life extension moiety is an antibody Fc domain. In some embodiments, the half-life extension moiety is HSA. In some embodiments, the half-life extension moiety is an HSA-binding recombinantly engineered variant of stefin polypeptide (i.e., AFFIMER® polypeptide), optionally comprising an amino acid sequence that has at least 70%, at least 80%, at least 90%, or 100% identity to an amino acid sequence of any one of SEQ ID NOS: 110-132.

### BRIEF DESCRIPTION OF THE DRAWINGS

FIG. 1 is a schematic illustrating an embodiment of the extending half-life therapeutic conjugates described herein. In the diagram, “X” represents a serum half-like extension moiety, in this case a protein linked through a maleimide conjugate with the thiol sidechain of a cysteine residue, to a linker including an FAPα substrate sequence to a drug moiety (the I-DASH inhibitor valine-boroproline shown) that is released upon FAP cleavage of the linker.

FIG. 2 is a graph showing the anti-tumor activity of examples of the therapeutic conjugates described herein, as indicated by tumor volume in a mouse model of colon carcinoma. The “SQT-Gly V.2-6325” groups are therapeutic conjugates without cell-targeting moieties. The “AVA04-182” groups comprise cell-targeting moieties.

FIG. 3 is a graph showing the amount of free Val-boroPro (talabostat) in tumors and in serum after administration of the therapeutic conjugates at the dosage levels shown.

FIGS. 4A-4B are graphs showing the EC₅₀ of talabostat administered alone (FIG. 4A) and administered with the therapeutic conjugates described herein (FIG. 4B) in mice and rats.

FIGS. 5A-5D are graphs depicting tumor volume and percent change in body weight in mice after being administered the vehicle (FIG. 5A), a conjugate comprising a cell-binding moiety (FIG. 5B), and a therapeutic conjugate described herein (the conjugate of FIG. 5B without the cell-binding moiety) (FIG. 5C). The percent change in body weight of the mice in FIG. 5C was also measured (FIG. 5D).

FIG. 6A is the structure for “6325”, an exemplary FAP-activated I-DASH inhibitor including an NHS group for conjugation to lysine residues of proteins. FIG. 6B is the structure for “6323”, an exemplary FAP-activated I-DASH inhibitor including a maleimide group for conjugation to lysine residues of proteins. FIG. 6C is the structure for “6501”, an exemplary tetra-branched FAP-activated I-DASH inhibitor including a maleimide group for conjugation to lysine residues of proteins.

FIG. 7 is a graph showing the change in tumor volume over time in CT26-mFAP+ mice with either vehicle (control), MSA-6325 (mouse serum albumin conjugated with 6325) and PEG-6325 (6325 reacted with SUNBRIGHT PTE-200 PA, a 20 KDa 4 arm functional PEG).

FIGS. 8A-8B are graphs showing the individual animal tumor volume growth curves over time in CT26-mFAP+mice with either a human Fc fragment conjugated with 6325 (FIG. 8A), or a full human IgG antibody conjugated with 6325 (FIG. 8B).

FIG. 9 illustrates an exemplary generic structure for an FAP-activated somatostatin analog (octreotide)-based conjugate, wherein X is the half-life extension moiety, L1 is a spacer or bond, X1 and X2 are independently O or S, R2 is hydrogen or (C1-C6) alkyl or hydrogen, R3 is hydrogen or a branched or straight chain lower alkyl, e.g., a lower alkyl such as methyl, R4 is a branched or straight chain lower alkoxy, such as hydroxymethyl (e.g., for serine) or 1-hydroxyethyl (e.g., for threonine) (in one embodiment, R4 is hydroxymethyl), L3 is a bond or a cleavable or non-cleavable linker, and Z is a cytotoxic, cytostatic or epigenetic moiety or a radioisotope containing moiety.

FIG. 10 illustrates an exemplary generic structure for an FAP-activated folic acid-based conjugate, wherein X is the half-life extension moiety, L1 is a spacer or bond, X1 and X2 are independently O or S, R2 is hydrogen or (C1-C6) alkyl or hydrogen, R3 is hydrogen or a branched or straight chain lower alkyl, e.g., a lower alkyl such as methyl, R4 is a branched or straight chain lower alkoxy, such as hydroxymethyl (e.g., for serine) or 1-hydroxyethyl (e.g., for threonine) (in one embodiment, R4 is hydroxymethyl), X3 is O or N(H), L3 is a bond or a cleavable or non-cleavable linker, and Z is a cytotoxic, cytostatic or epigenetic moiety or a radioisotope containing moiety.

FIG. 11 is an illustration of FAP-activated folic acid-based SMDC vintafolide, which is comprised of an FAP substrate recognition sequence (showing linker for protein conjugation), a folate targeting ligand, a peptide spacer, a self-immolative disulfide linker and the cytotoxic drug desacetyl vinblastine monohydrazine (DAVLBH).

FIG. 12 is an illustration of a FAP-activated folate-taxoid conjugate which incorporates an FAP substrate recognition sequence (showing linker for protein conjugation), a folic acid targeting moiety, a self-immolative disulfide linker and hydrophilic PEGylated dipeptide spacer (solubilising spacer) and a taxoid SB-T-1214, which is a derivative of the chemotherapeutic drug Taxol (see Seitz et al. Bioorg. Med. Chem., 2015, 23, 2187-2194).

FIGS. 13A-13B illustrate the structures of FAP-activated folate-doxorubicin (FIG. 13A) and thioloate (FIG. 13B) HDAC inhibitors.

FIG. 14 shows a FAP-activated folate-cytotix maytansinoid conjugate which incorporates an FAP substrate recognition sequence (showing linker for protein conjugation), a folic acid targeting moiety, a self-immolative disulfide linker and an antitubulin cytotoxic maytansinoid drug drug moiety DM4.

FIG. 15 illustrates the structure of FAP-activated folate-MMAE.

FIG. 16 illustrates the structure of camptothecin.

FIG. 17 includes graphs showing the fold-increase in lactate dehydrogenase (LDH, a marker of pyroptosis) released into culture supernatants from J774A.1 mouse macrophage cell line cells cultured with one of various versions of the FAP-activated I-DASH inhibitor 6325 (Hu IgG1 Fc-6325, Hu IgG1-6325, MSA-6325, SQT-Gly V.2-6325, SQT-Gly CF-6325, or SQT-Gly CG-6325) in the presence (open bar) or absence (grey filled bar) of rhFAPα for 24 hours. An untreated control was used for each set of experiments, and a Val-boroPro (VbP) control experiment was also performed (data not shown) All values are representative of the mean+/−SEM. Statistical analysis of No FAPα vs. +FAPα was performed via multiple T test in GraphPad Prism. *, P<0.05; **, P<0.01; ***, P<0.001.

FIGS. 18A-18B are graphs showing the conjugation of thiol (SH) groups determined by measurement of free SH of to compound maleimide linker prodrugs, 6323 (FIG. 18A) and 6501 (FIG. 18B).

FIGS. 19A-19B are graphs showing the pharmacokinetics of VbP released from Hu IgG1 Fc-6325 in CT26-mFAP tumor-bearing mouse serum (FIG. 19A) and tumor (FIG. 19B) in the presence or absence of 5057, a FAPα-specific inhibitor. Each time point includes three mice per group.

FIGS. 20A-20B are graphs showing the efficacy of SQT-Gly conjugates (FIG. 20A) and Hu IgG1 Fc conjugates (FIG. 20B), measured as tumor volume over time in a syngeneic murine colon cancer model (CT26-mFAP+mice).

FIG. 21 includes graphs showing the pharmacokinetics and tissue distribution of VbP released from different FAP-activated prodrugs in mouse serum and tumor in a murine colon cancer model (CT26-mFAP⁺ mice). Measurements were taking using liquid chromatography-mass spectrometry (LC-MS). Each dosing group consisted of a subset of 5 groups, representing the 1, 4, 6, 24, and 48 hour collection time points (n=3/group) with the exception of 24 μg 3892, which only consisted of 1, 4, and 24 hours.

FIGS. 22A-22B include graphs showing the pharmacokinetics and tissue distribution of VbP released from SQT-Gly V.2-conjugates (FIG. 22A) and IgG Fc-conjugates (FIG. 22B) in mouse serum and tumor in a murine colon cancer model (CT26-mFAP+mice). Measurements were taking using LC-MS. Each dosing group consisted of a subset of 5 groups, representing the 1, 4, 6, 24, and 48 hour collection time points (n=3/group).

FIG. 23 is a graph depicting FAP-activated prodrug G-CSF serum cytokine response in nontumor-bearing BALB/c mice levels. Levels of G-CSF in mouse serum following administration of vehicle (PBS), 42CQ-6501 and 42CQ-6501+MSA at 200 ug/mouse are shown.

FIGS. 24A-24B are graphs showing the conjugation of a compound maleimide linker prodrug (6323) to the hinge region cysteine residues of recombinantly engineered variant of stefin polypeptide (AFFIMER®)-Fc proteins. Conjugations were performed with reaction ratios of 0, 10, 20 and 40 moles of 6323 per mole of reduced SH (4 SH per SQTGlyCF). The change in free SH groups vs. reaction ratio is shown in FIG. 24A. A timed conjugation reaction was done with a reaction ratio of 40 6323 per SH with the reaction stopped at 0, 5, 10, 15 and 20 minutes after addition of 6323. Kinetics of conjugation of 6323 to SQTGlyCF with a reaction ratio of 40 6323 per SH group are shown in FIG. 24B.

FIGS. 25A-25B are graphs showing the pharmacokinetics of VbP released following administration of 42CQ-6501 in CT26-mFAP tumor-bearing mice. Each dosing group consisted of a subset of 5 groups, representing the 1, 4, 6, 24 and 48 hr collection time points (n=3/group). VbP was measured with LC-MS in serum (FIG. 25A) and tumor (FIG. 25B) samples.

FIGS. 26A-26B are graphs showing the efficacy of 42CQ-6323 conjugates (FIG. 26A) and 42CQ-6501 conjugates (FIG. 26B) ±MSA, measured as tumor volume over time in a syngeneic murine colon cancer model (CT26-mFAP+mice).

FIGS. 27A-27B are graphs showing the pharmacokinetics of VbP released following administration of 42CQ-6323 conjugates (FIG. 27A) and 42CQ-6501 conjugates (FIG. 27B) in wild-type and FAPα knockout mice (n=3/group).

FIG. 28 is the structure for “3892”, an exemplary prodrug of VbP.

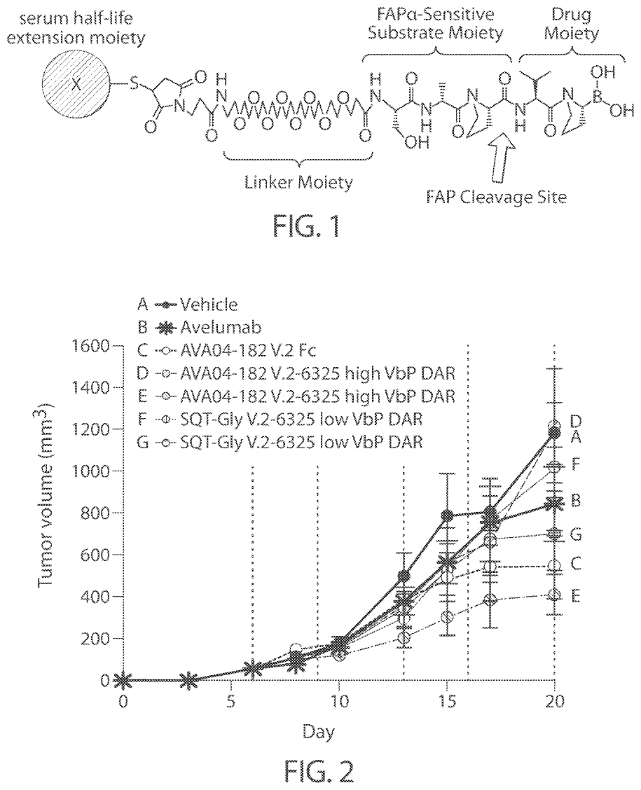

### DETAILED DESCRIPTION

The data provided herein demonstrates, unexpectedly, that the therapeutic conjugates of the present disclosure can be delivered systemically, without a cell-binding (e.g., cell targeting) moiety, yet are still capable of delivering a therapeutically effective amount of a therapeutic moiety (e.g., drug) to a target diseased tissue, while maintaining a therapeutic index that is superior to that of the therapeutic moiety delivered directly (e.g., by the same route of administration). Instead of a cell-binding moiety, the therapeutic conjugates described herein comprise a half-life extension moiety and an FAPα-cleavable linker, which when combined, enable local accumulation and release of the therapeutic moiety in a tumor microenvironment (or other target tissue expressing FAPα) while reducing exposure of non-target tissue that expresses FAPα at lower levels of than the target tissue. Accordingly, the therapeutic conjugates provided herein demonstrate one or more of (1) superior efficacy and/or ability to reach a higher percentage of maximum effective concentrations of the released therapeutic moiety in the target tissue due to localized FAPα cleavage of the prodrug and extended circulating serum half-lives, and (2) an improved therapeutic index (TI) due to the ability to be administered at a lower systemic Cmax concentration and/or reduced systemic toxicity relative to the parent therapeutic moiety.

Described herein are therapeutic conjugates comprising a drug moiety linked through an enzyme-cleavable linker to a half-life extension moiety. Each component of these conjugates is described in detail below.

### Half-Life Extension Moieties

A therapeutic conjugate, in some embodiments, comprises a half-life extension moiety. The half-life extension moiety extends the circulating serum half-life of a drug moiety in vivo. Circulating serum half-life is the amount of time it takes for the concentration or amount of the drug moiety in the serum to be reduced by half (50%). Half-life times can be determined experimentally by measuring the concentration of the drug moiety in the serum of a subject over time. Half-life may be calculated using the formula: 0.693×(Vd/CL), where Vd represents the volume of distribution and CL represents clearance. The volume of distribution is the theoretical volume needed to contain the total amount of the drug moiety at the same concentration that is observed in the blood plasma (e.g., the ratio of the amount of the drug moiety in the body to the concentration of the drug moiety measured in blood, plasma, and in free form in interstitial fluid). Clearance is the volume of plasma from which the drug moiety is completely removed per unit time. Half-life may also be determined using high performance liquid chromatography (HPLC), fluorescence assays, radioassays, radioimmunoassays, and elemental mass spectrometric assays.

A half-life extension moiety may extend the circulating serum half-life of a molecule by at least 2-fold, relative to the circulating serum half-life of the molecule not linked to the half-life extension moiety. In some embodiments, a half-life extension moiety extends the circulating serum half-life of a molecule by at least 3-fold, at least 4-fold, at least 5-fold, at least 6-fold, at least 7-fold, at least 8-fold, at least 9-fold, at least 10-fold, at least 20-fold, or at least 30-fold, relative to the circulating serum half-life of the molecule not linked to the half-life extension moiety. In some embodiments, a half-life extension moiety extends the circulating serum half-life of a molecule by 2-fold to 5-fold, 2-fold to 10-fold, 3-fold to 5-fold, 3-fold to 10-fold, 15-fold to 5-fold, 4-fold to 10-fold, or 5-fold to 10-fold, relative to the circulating serum half-life of the molecule not linked to the half-life extension moiety.

In some embodiments, a half-life extension moiety extends the circulating serum half-life of a drug moiety by at least 10 hours, at least 12 hours, at least 15 hours, at least 20 hours, at least 24 hours, at least 48 hours, at least 72 hours, or at least 96 hours, for example, at least 1 week after in vivo administration, relative to the circulating serum half-life of the drug moiety not linked to the half-life extension moiety.

In some embodiments, the drug moieties have a circulating serum half-life in human subjects of at least 10 hours. In some embodiments, the drug moieties have a serum half-life in human subjects of at least 24 hours. In some embodiments, the drug moieties have a serum half-life in human subjects of at least 48 hours. In some embodiments, the drug moieties have a serum half-life in human subjects of at least 72 hours. In some embodiments, the drug moieties have a serum half-life in human subjects of at least 96 hours. In some embodiments, the drug moieties have a serum half-life in human subjects of at least 120 hours. In some embodiments, the drug moieties have a serum half-life in human subjects of at least 144 hours. In some embodiments, the drug moieties have a serum half-life in human subjects of at least 168 hours. In some embodiments, the drug moieties have a serum half-life in human subjects of at least 192 hours. In some embodiments, the drug moieties have a serum half-life in human subjects of at least 216 hours. In some embodiments, the drug moieties have a serum half-life in human subjects of at least 240 hours. In some embodiments, the drug moieties have a serum half-life in human subjects of at least 264 hours. In some embodiments, the drug moieties have a serum half-life in human subjects of at least 288 hours. In some embodiments, the drug moieties have a serum half-life in human subjects of at least 312 hours. In some embodiments, the drug moieties have a serum half-life in human subjects of at least 336 hours. In some embodiments, the drug moieties have a serum half-life in human subjects of at least 360 hours. In some embodiments, the drug moieties have a serum half-life in human subjects of 24 to 360 hours, 48 to 360 hours, 72 to 360 hours, 96 to 360 hours, or 120 to 360 hours.

Serum proteins and antibody Fe domains are non-limiting examples of two major proteins that may be used as provided herein as half-life extension moieties. Both Fc and serum protein conjugates achieve extended half-lives not only by increasing the size of the drug moiety, but both also take advantage of the body's natural recycling mechanism: the neonatal Fc receptor, FcRn. The pH-dependent binding of these proteins to FcRn prevents degradation of the therapeutic conjugate in the endosome. Conjugates using these proteins can have half-lives in the range of 3-16 days. Conjugates that include an antibody Fc domain or a serum protein can improve the solubility and stability of the drug moiety.

Serum Proteins

In some embodiments, the half-life extension moiety comprises a serum protein (e.g., a naturally-occurring or modified version of a protein in blood (e.g., serum) of a human). The serum protein may be, for example, fibronectin, transferrin, or albumin.

In some embodiments, the serum protein is fibronectin. Fibronectin is a high-molecular weight (˜440 kDa) glycoprotein of the extracellular matrix that binds to membrane-spanning receptor proteins called integrins. It exists as a protein dimer, including two nearly identical polypeptide chains linked by a pair of C-terminal disulfide bonds. Each fibronectin subunit has a molecular weight of 230-250 kDa and contains three types of modules: type I, II, and III. All three modules are composed of two anti-parallel β-sheets resulting in a Beta-sandwich; however, type I and type II are stabilized by intra-chain disulfide bonds, while type III modules do not contain any disulfide bonds. The absence of disulfide bonds in type III modules allows them to partially unfold under applied force. Fibronectin also binds to other extracellular matrix proteins such as collagen, fibrin, and heparan sulfate proteoglycans (e.g. syndecans). Fibronectin exists as a protein dimer, consisting of two nearly identical monomers linked by a pair of disulfide bonds. The fibronectin protein is produced from a single gene, but alternative splicing of its pre-mRNA leads to the creation of several isoforms.

In some embodiments, the serum protein is transferrin. Trasnferrin is a glycoprotein found in vertebrates that binds to and consequently mediates the transport of Tron (Fe) through blood plasma. It is produced in the liver and contains binding sites for two Fe³⁺ atoms. Human transferrin is encoded by the TF gene and produced as a 76 kDa glycoprotein. Transferrin glycoproteins bind iron tightly, but reversibly. Although iron bound to transferrin is less than 0.1% (4 mg) of total body iron, it forms the most vital iron pool with the highest rate of turnover (25 mg/24 h). Transferrin has a molecular weight of around 80 kDa and contains two specific high-affinity Fe(III) binding sites. The affinity of transferrin for Fe(III) is extremely high (association constant is 1020 M⁻¹ at pH 7.4) but decreases progressively with decreasing pH below neutrality. Transferrins are not limited to only binding to iron but also to different metal ions.

In some embodiments, the serum protein is human serum albumin (HSA). HSA is a protein encoded by the ALB gene. HSA is a 585 amino acid polypeptide (approx. 67 kDa) having a serum half-life of about 20 days and is primarily responsible for the maintenance of colloidal osmotic blood pressure, blood pH, and transport and distribution of numerous endogenous and exogenous ligands. HSA has three structurally homologous domains (domains I, II and III), is almost entirely in the alpha-helical conformation, and is highly stabilized by 17 disulfide bridges. A representative HSA sequence is provided by UniProtKB Primary accession number P02768 and may include other human isoforms thereof.

Serum Protein Binding Molecules

In some embodiments, the half-life extension moiety comprises a molecule that binds to a serum protein, such as proteins that bind non-covalently via a peptide or protein-binding domain to the serum protein, such as an antibody (including antigen-binding portions thereof). Examples of antibodies include, but are not limited to: Fab, F(ab)₂, F(ab′), F(ab′)₂, F(ab′)₃, Fd, Fv, disulfide linked Fv, dAb or sdAb (or NANOBODY®), CDR, scFv, (scFv)₂, di-scFv, bi-scFv, tascFv (tandem scFv), AVIBODY (e.g., diabody, triabody, and tetrabody), T-cell engager (BiTE), Fc, scFv-Fc, Fcab, mAb2, small modular immunopharmaceutical (SMIP), Genmab/unibody or duobody, V-NAR domain, IgNAR, minibody, IgGACH2, DVD-Ig, probody, intrabody, and a multispecificity antibody. Other examples are provided in US 2005/0287153, incorporated herein by reference in its entirety.

In some embodiments, the serum protein-binding molecule is hydrophobic moiety that associates with serum albumin, such as acylated peptides (e.g., acylated heptapeptide in Zorzi et al., Nature Communications vol 8: 16092 (2017); the acyl group of Liraglutide).

In some embodiments, the molecule that binds to a serum protein is a non-antibody molecule, non-limiting examples of which include an affibody, an AFFIMER® polypeptide, an affilin, an anticalin, an atrimer, an avimer, a DARPin, an FN3 scaffold (e.g., Adnectins, Centyrins), a fynomer, a Kunitz domain, a nanofitin, a pronectins, a tribody, bicyclic peptides, and a Cys-knot.

In some embodiments, the molecule that binds a serum protein such as HSA comprises an AFFIMER® polypeptide. An AFFIMER® polypeptide is a small, highly stable polypeptide (e.g., protein) that is a recombinantly engineered variant of stefin polypeptides. Thus, the term “AFFIMER® polypeptide” may be used interchangeably herein with the term “recombinantly engineered variant of stefin polypeptide.” A stefin polypeptide is a subgroup of proteins in the cystatin superfamily—a family that encompasses proteins containing multiple cystatin-like sequences. The stefin subgroup of the cystatin family is relatively small (˜100 amino acids) single domain proteins. They receive no known post-translational modification, and lack disulfide bonds, suggesting that they will be able to fold identically in a wide range of extracellular and intracellular environments. Stefin A is a monomeric, single chain, single domain protein of 98 amino acids. The structure of stefin A has been solved, facilitating the rational mutation of stefin A into the AFFIMER® polypeptide. The only known biological activity of cystatins is the inhibition of cathepsin activity, has enabled exhaustively testing for residual biological activity of the engineered proteins.

An AFFIMER® polypeptide displays two peptide loops and an N-terminal sequence that can all be randomized to bind to desired target proteins with high affinity and specificity, in a similar manner to monoclonal antibodies. Stabilization of the two peptides by the stefin A protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the stefin A polypeptide sequence can be carried out, with such variations improving the properties of these affinity reagents, such as increase stability, make them robust across a range of temperatures and pH, for example. In some embodiments, an AFFIMER® polypeptide includes a sequence derived from stefin A, sharing substantial identify with a stefin A wild type sequence, such as human stefin A. In some embodiments, an AFFIMER® polypeptide has an amino acid sequence that shares at least 25%, 35%, 45%, 55% or 60% identity to the sequences corresponding to human stefin A. For example, an AFFIMER® polypeptide may have an amino acid sequence that shares at least 70%, at least 80%, at least 85%, at least 90%, at least 92%, at least 94%, at least 95% identity, e.g., where the sequence variations do not adversely affect the ability of the scaffold to bind to the desired target, and e.g., which do not restore or generate biological functions such as those that are possessed by wild type stefin A, but which are abolished in mutational changes described herein.

An anti-HSA AFFIMER® polypeptide comprises an AFFIMER® polypeptide in which at least one of the solvent accessible loops is from the wild-type stefin A protein having amino acid sequences to enable an AFFIMER® polypeptide to bind HSA, selectively, and in some embodiments, with K_{d} of 10⁻⁶M or less.

In some embodiments, the AFFIMER® polypeptide binds to HSA with a K_{d} of 1×10⁻⁹ M to 1×10⁻⁶ M at pH 7.4 to 7.6. In some embodiments, the AFFIMER® polypeptide binds to HSA with a K_{d} of 1×10⁻⁶ M or less at pH 7.4 to 7.6. In some embodiments, the AFFIMER® polypeptide binds to HSA with a K_{d} of 1×10⁻⁷ M or less at pH 7.4 to 7.6. In some embodiments, the AFFIMER® polypeptide binds to HSA with a K_{d} of 1×10⁻⁸ M or less at pH 7.4 to 7.6. In some embodiments, the AFFIMER® polypeptide binds to HSA with a K_{d} of 1×10⁻⁹ M or less at pH 7.4 to 7.6. In some embodiments, the AFFIMER® polypeptide binds to HSA with a K_{d} of 1×10⁻¹⁰ M or less at pH 7.4 to 7.6. In some embodiments, the AFFIMER® polypeptide binds to HSA with a K_{d} of 1×10⁻¹¹ M or less at pH 7.4 to 7.6. In some embodiments, the AFFIMER® polypeptide binds to HSA with a K_{d} of 1×10⁻⁹ M to 1×10⁻⁶ M at pH 7.4. In some embodiments, the AFFIMER® polypeptide binds to HSA with a K_{d} of 1×10⁻⁶ M or less at pH 7.4. In some embodiments, the AFFIMER® polypeptide binds to HSA with a K_{d} of 1×10⁻⁷ M or less at pH 7.4. In some embodiments, the AFFIMER® polypeptide binds to HSA with a K_{d} of 1×10⁻⁸ M or less at pH 7.4. In some embodiments, the AFFIMER® polypeptide binds to HSA with a K_{d} of 1×10⁻⁹ M or less at pH 7.4. In some embodiments, the AFFIMER® polypeptide binds to HSA with a K_{d} of 1×10⁻¹⁰ M or less at pH 7.4. In some embodiments, the AFFIMER® polypeptide binds to HSA with a K_{d} of 1×10⁻¹¹ M or less at pH 7.4.

In some embodiments, the anti-HSA AFFIMER® polypeptide is derived from the wild-type human stefin A protein having a backbone sequence and in which one or both of loop 2 (designated (Xaa)ₙ) and loop 4 (designated (Xaa)ₘ) are replaced with alternative loop sequences (Xaa)ₙ and (Xaa)ₘ, to have the general formula (I):

FR1-(Xaa)n-FR2-(Xaa)m-FR3 (I),

wherein FR1 is an amino acid sequence having at least 70% identity to MIPGGLSEAK PATPEIQEIV DKVKPQLEEK TNETYGKLEA VQYKTQVLA (SEQ ID NO: 1); FR2 is an amino acid sequence having at least 70% identity to GTNYYIKVRA GDNKYMHLKV FKSL (SEQ ID NO: 2); FR3 is an amino acid sequence having at least 70% identity to EDLVLTGYQV DKNKDDELTG F (SEQ ID NO: 3); Xaa, individually for each occurrence, is an amino acid; and n is an integer from 3 to 20, and m is an integer from 3 to 20.

In some embodiments, FR1 is a polypeptide sequence having 80%-98%, 82%-98%, 84%-98%, 86%-98%, 88%-98%, 90%-98%, 92%-98%, 94%-98%, or 96%-98% homology with SEQ ID NO: 1. In some embodiments, FR1 is a polypeptide sequence having 80%, 82%, 84%, 86%, 88%, 90%, 92%, 94%, 96%, or 95% homology with SEQ ID NO: 1. In some embodiments, FR1 is the polypeptide sequence of SEQ ID NO: 1. In some embodiments, FR2 is a polypeptide sequence having at least 80%-96%, 84%-96%, 88%-96%, or 92%-96% homology with SEQ ID NO: 2. In some embodiments, FR2 is a polypeptide sequence having at least 80%, 84%, 88%, 92%, or 96% homology with SEQ ID NO: 2. In some embodiments, FR2 is a polypeptide sequence having at least 80%, 85%, 90%, 95% or even 98% identity with SEQ ID NO: 2. In some embodiments, FR2 is the polypeptide sequence of SEQ ID NO: 2. In some embodiments, FR3 is a polypeptide sequence having at least 80%-95%, 85%-95%, or 90%-95% homology with SEQ ID No: 3. In some embodiments, FR3 is a polypeptide sequence having at least 80%, 85%, 90%, or 95% homology with SEQ ID NO: 3. In some embodiments, FR3 is the polypeptide sequence of SEQ ID NO: 3.

In some embodiments, an anti-HSA AFFIMER® polypeptide comprises the amino acid sequence represented in general formula (II):

(II)
(SEQ ID NO: 4)
MIP-Xaa1-GLSEAKPATPEIQEIVDKVKPQLEEKTNETYGKLEAVQYK
TQVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF,

wherein Xaa, individually for each occurrence, is an amino acid; n is an integer from 3 to 20, and m is an integer from 3 to 20; Xaa1 is Gly, Ala, Val, Arg, Lys, Asp, or Glu; Xaa2 is Gly, Ala, Val, Ser or Thr; Xaa3 is Arg, Lys, Asn, Gln, Ser, Thr; Xaa4 is Gly, Ala, Val, Ser or Thr; Xaa5 is Ala, Val, Ile, Leu, Gly or Pro; Xaa6 is Gly, Ala, Val, Asp or Glu; and Xaa7 is Ala, Val, Ile, Leu, Arg or Lys.

In some embodiments, Xaa1 is Gly, Ala, Arg or Lys. In some embodiments, Xaa1 is Gly or Arg. In some embodiments, Xaa2 is Gly, Ala, Val, Ser or Thr. In some embodiments, Xaa2 is Gly or Ser. In some embodiments, Xaa3 is Arg, Lys, Asn, Gln, Ser, Thr. In some embodiments, Xaa3 is Arg, Lys, Asn or Gln. In some embodiments, Xaa3 is Lys or Asn. In some embodiments, Xaa4 is Gly, Ala, Val, Ser or Thr. In some embodiments, Xaa4 is Gly or Ser. In some embodiments, Xaa5 is Ala, Val, Ile, Leu, Gly or Pro. In some embodiments, Xaa5 is Ile, Leu or Pro. In some embodiments, Xaa5 is Leu or Pro. In some embodiments, Xaa6 is Gly, Ala, Val, Asp or Glu. In some embodiments, Xaa6 is Ala, Val, Asp or Glu. In some embodiments, Xaa6 is Ala or Glu. In some embodiments, Xaa7 is Ala, Val, Ile, Leu, Arg or Lys. In some embodiments, Xaa7 is Ile, Leu or Arg. In some embodiments, Xaa7 is Leu or Arg.

In some embodiments, an anti-HSA AFFIMER® comprises the amino acid sequence represented in general formula (III):

(III)
(SEQ ID NO: 5)
MIPRGLSEAKPATPEIQEIVDKVKPQLEEKTNETYGKLEAVQYKTQVLA-
(Xaa)n-STNYYIKVRAGDNKYMHLKVFNGP-(Xaa)m-ADRVLTGYQVD
KNKDDELTGF,

wherein Xaa, individually for each occurrence, is an amino acid; n is an integer from 3 to 20, and m is an integer from 3 to 20. In some embodiments, n is 3, 4, 5, 6, 7, 8, 9, 10, 11, 12, 13, 14, 15, 16, 17, 18, 19, or 20. In some embodiments, n is 8 to 10, 7 to 11, 6 to 12, 5 to 13, 4 to 14, or 3 to 15. In some embodiments, m is 3, 4, 5, 6, 7, 8, 9, 10, 11, 12, 13, 14, 15, 16, 17, 18, 19, or 20. In some embodiments, m is 8 to 10, 7 to 11, 6 to 12, 5 to 13, 4 to 14, or 3 to 15.

In some embodiments, (Xaa)ₙ is represented by formula (IV):

aa1-aa2-aa3-aa4-aa5-aa6-aa7-aa8-aa9 (IV),

wherein aa1 is an amino acid with a neutral polar hydrophilic side chain; aa2 is an amino acid with a neutral nonpolar hydrophobic side chain; aa3 is an amino acid with a neutral nonpolar hydrophobic side chain; aa4 is an amino acid with a neutral polar hydrophilic side chain; aa5 is an amino acid with a positively charged polar hydrophilic side chain; aa6 is an amino acid with a positively charged polar hydrophilic side chain; aa7 is an amino acid with a neutral nonpolar hydrophobic side chain; aa8 is an amino acid with a neutral nonpolar hydrophobic side chain; and aa9 is an amino acid with a neutral nonpolar hydrophilic side chain.

In some embodiments, (Xaa)ₘ is represented by formula (V):

aa1-aa2-aa3-aa4-aa5-aa6-aa7-aa8-aa9 (V),

wherein aa1 is an amino acid with a neutral nonpolar hydrophobic side chain; aa2 is an amino acid with a positively charged polar hydrophilic side chain; aa3 is an amino acid with a neutral nonpolar hydrophobic side chain; aa4 is an amino acid with a positively charged polar hydrophilic side chain; aa5 is an amino acid with a neutral polar hydrophilic side chain; aa6 is an amino acid with a neutral polar hydrophilic side chain; aa7 is an amino acid with a negatively charged polar hydrophilic side chain; aa8 is an amino acid with a positively charged polar hydrophilic side chain; and aa9 is an amino acid with a neutral nonpolar hydrophilic side chain.

Examples of amino acids with a neutral nonpolar hydrophilic side chain include cysteine (Cys) and glycine (Gly). In some embodiments, the amino acid with a neutral nonpolar hydrophilic side chain is Cys. In some embodiments, the amino acid with a neutral nonpolar hydrophilic side chain is Gly.

Examples of amino acids with a neutral nonpolar hydrophobic side chain include alanine (Ala), isoleucine (Ile), leucine (Leu), methionine (Met), phenylalanine (Phe), proline (Pro), tryptophan (Trp), and valine (Val). In some embodiments, the amino acid with a neutral nonpolar hydrophobic side chain is Ala. In some embodiments, the amino acid with a neutral nonpolar hydrophobic side chain is Ile. In some embodiments, the amino acid with a neutral nonpolar hydrophobic side chain is Leu. In some embodiments, the amino acid with a neutral nonpolar hydrophobic side chain is Met. In some embodiments, the amino acid with a neutral nonpolar hydrophobic side chain is Phe. In some embodiments, the amino acid with a neutral nonpolar hydrophobic side chain is Pro. In some embodiments, the amino acid with a neutral nonpolar hydrophobic side chain is Trp. In some embodiments, the amino acid with a neutral nonpolar hydrophobic side chain is Val.

Examples of amino acids with a neutral polar hydrophilic side chain include asparagine (Asn), glutamine (Gln), serine (Ser), threonine (Thr), and tyrosine (Tyr). In some embodiments, the amino acid with a neutral polar hydrophilic side chain is Asn. In some embodiments, the amino acid with a neutral polar hydrophilic side chain is Gln. In some embodiments, the amino acid with a neutral polar hydrophilic side chain is Ser. In some embodiments, the amino acid with a neutral polar hydrophilic side chain is Thr. In some embodiments, the amino acid with a neutral polar hydrophilic side chain is Tyr.

Examples of amino acids with a positively charged polar hydrophilic side chain include arginine (Arg), histidine (His), and lysine (Lys). In some embodiments, the amino acid with a positively charged polar hydrophilic side is Arg. In some embodiments, the amino acid with a positively charged polar hydrophilic side is His. In some embodiments, the amino acid with a positively charged polar hydrophilic side is Lys.

Examples of amino acids with a negatively charged polar hydrophilic side chain include aspartate (Asp) and glutamate (Glu). In some embodiments, the amino acid with a negatively charged polar hydrophilic side chain is Asp. In some embodiments, the amino acid with a negatively charged polar hydrophilic side chain is Glu.

In some embodiments, (Xaa)ₙ is represented by formula (IV):

aa1-aa2-aa3-aa4-aa5-aa6-aa7-aa8-aa9 (IV),

wherein aa1 is an amino acid selected from Asp, Gly, Asn, and Val; aa2 is an amino acid selected from Trp, Tyr, His, and Phe; aa3 is an amino acid selected from Trp, Tyr, Gly, Trp, and Phe; aa4 is an amino acid selected from Gln, Ala, and Pro; aa5 is an amino acid selected from Ala, Gln, Glu, Arg, and Ser; aa6 is an amino acid selected from Lys, Arg, and Tyr; aa7 is an amino acid selected from Trp and Gln; aa8 is an amino acid selected from Pro and His; and/or aa9 is an amino acid selected from His, Gly, and Gln. In some embodiments, aa1 is Asp. In some embodiments, aa1 is Gly. In some embodiments, aa1 is Asn. In some embodiments, aa2 is Trp. In some embodiments, aa2 is Tyr. In some embodiments, aa2 is His. In some embodiments, aa2 is Phe. In some embodiments, aa3 is Trp. In some embodiments, aa3 is Tyr. In some embodiments, aa3 is Gly. In some embodiments, aa3 is Trp. In some embodiments, aa3 is Phe. In some embodiments, aa4 is Gln. In some embodiments, aa4 is Ala. In some embodiments, aa4 is Pro. In some embodiments, aa5 is Ala. In some embodiments, aa5 is Gln. In some embodiments, aa5 is Glu. In some embodiments, aa5 is Arg. In some embodiments, aa5 is Ser. In some embodiments, aa6 is Lys. In some embodiments, aa6 is Arg. In some embodiments, aa6 is Tyr. In some embodiments, aa7 is Trp. In some embodiments, aa7 is Gln. In some embodiments, aa8 is Pro. In some embodiments, aa8 is His. In some embodiments, aa9 is His. In some embodiments, aa9 is Gly. In some embodiments, aa9 is Gln.

In some embodiments, (Xaa)ₘ is represented by formula (IV):

aa1-aa2-aa3-aa4-aa5-aa6-aa7-aa8-aa9 (IV),

wherein aa1 is an amino acid selected from Tyr, Phe, Trp, and Asn; aa2 is an amino acid selected from Lys, Pro, His, Ala, and Thr; aa3 is an amino acid selected from Val, Asn, Gly, Gln, Ala, and Phe; aa4 is an amino acid selected from His, Thr, Lys, Trp, Lys, Val, and Arg; aa5 is an amino acid selected from Gln, Ser, Gly, Pro, and Asn; aa6 is an amino acid selected from Ser, Tyr, Glu, Leu, Lys, and Thr; aa7 is an amino acid selected from Ser, Asp, Val, and Lys; aa8 is an amino acid selected from Gly, Leu, Ser, Pro, His, Asp, and Arg; and/or aa9 is an amino acid selected from Gly, Gln, Glu, and Ala. In some embodiments, aa1 is Tyr. In some embodiments, aa1 is Phe. In some embodiments, aa1 is Trp. In some embodiments, aa1 is Asn. In some embodiments, aa2 is Lys. In some embodiments, aa2 is Pro. In some embodiments, aa2 is His. In some embodiments, aa2 is Ala. In some embodiments, aa2 is Thr. In some embodiments, aa3 is Val. In some embodiments, aa3 is Asn. In some embodiments, aa3 is Gly. In some embodiments, aa3 is Gln. In some embodiments, aa3 is Ala. In some embodiments, aa3 is Phe. In some embodiments, aa4 is His. In some embodiments, aa4 is Thr. In some embodiments, aa4 is Lys. In some embodiments, aa4 is Trp. In some embodiments, aa4 is Lys. In some embodiments, aa4 is Val. In some embodiments, aa4 is Arg. In some embodiments, aa5 is Gln. In some embodiments, aa5 is Ser. In some embodiments, aa5 is Gly. In some embodiments, aa5 is Pro. In some embodiments, aa5 is Asn. In some embodiments, aa6 is Ser. In some embodiments, aa6 is Tyr. In some embodiments, aa6 is Glu. In some embodiments, aa6 is Leu. In some embodiments, aa6 is Lys. In some embodiments, aa6 is Thr. In some embodiments, aa7 is Ser. In some embodiments, aa7 is Asp. In some embodiments, aa7 is Val. In some embodiments, aa7 is Lys. In some embodiments, aa8 is Gly. In some embodiments, aa8 is Leu. In some embodiments, aa8 is Ser. In some embodiments, aa8 is Pro. In some embodiments, aa8 is His. In some embodiments, aa8 is Asp. In some embodiments, aa8 is Arg. In some embodiments, aa9 is Gly. In some embodiments, aa9 is Gln. In some embodiments, aa9 is Glu. In some embodiments, aa9 is Ala.

In some embodiments, (Xaa)ₙ is represented by formula (V):

| | (V) |
|---|---|
| (SEQ ID NO: 140) | |
| | Asn-aa1-aa2-Gln-Gln-Arg-Arg-Trp-Pro-Gly, |

wherein aa1 is an amino acid selected from Trp and Phe; and aa2 is an amino acid selected from Tyr and Phe. In some embodiments, aa1 is Trp. In some embodiments, aa1 is Phe.

In some embodiments, aa2 is Tyr. In some embodiments, aa2 it Phe.

In some embodiments, (Xaa)ₙ is represented by formula (VI):

| | (VI) |
|---|---|
| (SEQ ID NO: 141) | |
| | aa1-aa2-Trp-aa3-aa4-Lys-Trp-Pro-aa5, |

wherein aa1 is an amino acid selected from Asp and Gly; aa2 is an amino acid selected from Trp, Tyr, and Phe; aa3 is an amino acid selected from Gln and Ala; aa4 is an amino acid selected from Ala and Ser; and aa5 is an amino acid selected from His and Gly. In some embodiments, aa1 is Asp. In some embodiments, aa1 is Gly. In some embodiments, aa2 is Trp. In some embodiments, aa2 is Tyr. In some embodiments, aa2 is Phe. In some embodiments, aa3 is Gln. In some embodiments, aa3 is Ala. In some embodiments, aa4 is Ala. In some embodiments, aa4 is Ser. In some embodiments, aa5 is His. In some embodiments, aa5 is Gly.

In some embodiments, (Xaa)ₙ is represented by formula (VII):

aa1-aa2-aa3-aa4-aa5-aa6-Trp-Pro-Gly (VII),

wherein aa1 is an amino acid selected from Gly and Asn; aa2 is an amino acid selected from Tyr, Phe, Trp, and His; aa3 is an amino acid selected from Trp, Tyr, and Phe; aa4 is an amino acid selected from Ala and Gln; aa5 is an amino acid selected from Ala, Ser, Gln, and Arg; and aa6 is an amino acid selected from Lys, Arg, and Tyr. In some embodiments, aa1 is Gly. In some embodiments, aa1 is Asn. In some embodiments, aa2 is Tyr. In some embodiments, aa2 is Phe. In some embodiments, aa2 is Trp. In some embodiments, aa2 is His. In some embodiments, aa3 is Trp. In some embodiments, aa3 is Tyr. In some embodiments, aa3 is Phe. In some embodiments, aa4 is Ala. In some embodiments, aa4 is Gln. In some embodiments, aa5 is Ala. In some embodiments, aa5 is Ser. In some embodiments, aa5 is Gln. In some embodiments, aa5 is Arg. In some embodiments, aa6 is Lys. In some embodiments, aa6 is Arg. In some embodiments, aa6 is Tyr.

In some embodiments, (Xaa)ₙ is represented by formula (IX):

| | (IX) |
|---|---|
| (SEQ ID NO: 142) | |
| | Gly-aa1-aa2-Ala-aa3-aa4-Trp-Pro-Gly, |

wherein aa1 is an amino acid selected from Tyr, Phe, and His; aa2 is an amino acid selected from Trp and Tyr; aa3 is an amino acid selected from Ala, Ser, and Arg; and aa4 is an amino acid selected from Lys and Tyr. In some embodiments, aa1 is Tyr. In some embodiments, aa1 is Phe His. In some embodiments, aa1 is His. In some embodiments, aa2 is Trp. In some embodiments, aa2 is Tyr. In some embodiments, aa3 is Ala. In some embodiments, aa3 is Ser. In some embodiments, aa3 is Arg. In some embodiments, aa4 is Lys. In some embodiments, aa4 is Tyr.

In some embodiments, (Xaa)ₙ is represented by formula (X):

aa1-aa2-aa3-Gln-aa4-aa5-Trp-Pro-aa6 (X),

wherein aa1 is an amino acid selected from Asp and Asn; aa2 is an amino acid selected from Trp and Phe; aa3 is an amino acid selected from Trp, Tyr, and Phe; aa4 is an amino acid selected from Ala, Gln, and Arg; aa5 is an amino acid selected from Lys and Arg; and aa6 is an amino acid selected from His and Gly. In some embodiments, aa1 is Asp. In some embodiments, aa1 is Asn. In some embodiments, aa2 is Trp. In some embodiments, aa2 is Phe. In some embodiments, aa3 is Trp. In some embodiments, aa3 is Tyr. In some embodiments, aa3 is Phe. In some embodiments, aa4 is Ala. In some embodiments, aa4 is Gln. In some embodiments, aa4 is Arg. In some embodiments, aa5 is Lys. In some embodiments, aa5 is Arg. In some embodiments, aa6 is His. In some embodiments, aa6 is Gly.

In some embodiments, an and -HSA AFFIMER® polypeptide comprises a loop 2 amino acid sequence selected from any one of SEQ ID NOS: 6-56, 134-135 (Table 1). In some embodiments, an anti-HSA AFFIMER® polypeptide comprises a loop 4 amino acid sequence selected from any one of SEQ ID NOS: 57-109 (Table 1).

TABLE 1
Examples of HSA AFFIMER ® Polypeptide
Loop Sequences

| | | SEQ ID | | SEQ ID |
|---|---|---|---|---|
| Name | Loop 2 | NO: | Loop 4 | NO: |
| HSA-00 | WTQPKNEHH | 134 | RFKYFAHYQ | 57 |
| HSA-01 | HLKHTDAQP | 135 | FHDFWHRRW | 58 |
| HSA-02 | HDQDVLHAW | 6 | DWYHYWWEV | 59 |
| HSA-03 | KFHRQEWAD | 7 | STRSIHVTT | 60 |
| HSA-04 | PEDFWDPEH | 8 | KQHHHYLDK | 61 |
| HSA-05 | VVRTTGHVV | 9 | HSAQDREIP | 62 |
| HSA-06 | YWWFCTGQS | 10 | WVQSGYNSQ | 63 |
| HSA-07 | IHHRQARSL | 11 | AVFWGKWSD | 64 |
| HSA-08 | SHRRRAYIW | 12 | QSFDKPWTT | 65 |
| HSA-09 | WDSHHWRAP | 13 | HYPLKYSFE | 66 |
| HSA-10 | DKRVKYGQ | 14 | WHHPWHRNR | 67 |
| HSA-11 | SDWVYALQL | 15 | DPWWAWVVW | 68 |
| HSA-12 | FWWFWY | 16 | FDNQDLIQY | 69 |
| HSA-13 | VRDWPWNTF | 17 | EKKNWYKWD | 70 |
| HSA-14 | QKKRDEDYI | 18 | DRHKSRWGI | 71 |
| HSA-15 | GVHEEPRKL | 19 | LNPFTPSVT | 72 |
| HSA-16 | EWWQKHWPS | 20 | YKGALLNHD | 73 |
| HSA-17 | NFFQRRWPG | 21 | WKFRNTERG | 74 |
| HSA-18 | DWWQAKWPH | 22 | YKVHQSSGG | 75 |
| HSA-19 | GIWQSRWPG | 23 | FHPIAGRPW | 76 |
| HSA-20 | GYWAAKWPG | 24 | FPNTSYDLQ | 77 |
| HSA-21 | GFYADHWPG | 25 | FAHYNLKSG | 78 |
| HSA-22 | NWYQQRWPG | 26 | WHNYGESSG | 79 |
| HSA-23 | GFYARHWPG | 27 | KFYYADHQW | 80 |
| HSA-24 | DFWKAHWPG | 28 | YTHADPHSQ | 81 |
| HSA-25 | DFYSVRWPG | 29 | FGVPQLGAG | 82 |
| HSA-26 | YWAANHASK | 30 | YSGFPFAGF | 83 |
| HSA-27 | IKRLEHWEY | 31 | WFSWPYTPL | 84 |
| HSA-28 | EWDSPWSEN | 32 | YYHPSIQST | 85 |
| HSA-29 | KHKNLRWPF | 33 | FLGWKDTVV | 86 |
| HSA-30 | RHFPKQTNW | 34 | DWWKWWWAK | 87 |
| HSA-31 | VWGPEYQHQ | 35 | NAGWPLVPE | 88 |
| HSA-32 | TWKNNGQDV | 36 | YALDPFGGK | 89 |
| HSA-33 | ATWLNYYLP | 37 | GYKFWGVSD | 90 |
| HSA-34 | DQESLFLNN | 38 | QGKQYILLR | 91 |
| HSA-35 | GFYAQHWPD | 39 | YKRHSAHDY | 92 |
| HSA-36 | GHYARYWPG | 40 | WAQKSKVHQ | 93 |
| HSA-37 | GFWASKWPG | 41 | FTAVSKKDA | 94 |
| HSA-38 | GFWQRKWPN | 42 | WGDKENIWF | 95 |
| HSA-39 | VWPADNDLK | 43 | WSGHPWVQK | 96 |
| HSA-40 | HWAWTSPGY | 44 | YADYPLSPK | 97 |
| HSA-41/ | NFFQRRWPG | 45 | WKFRNTDRG | 98 |
| HSA-52 | | | | |
| HSA-42 | HHSHRLKGQ | 46 | QTVATHYHY | 99 |
| HSA-43 | YQNTIFLSI | 47 | WHAKHLLSH | 100 |
| HSA-44 | FQDQFTWSQ | 48 | SGIKKADSV | 101 |
| HSA-45 | GEPHWPWQA | 49 | KANLINVKS | 102 |
| HSA-46 | ADPRHPWVE | 50 | WKSHVEVRS | 103 |
| HSA-47 | FHKRFQSQG | 51 | WVTQKYIIQ | 104 |
| HSA-48 | EWWQNRWPN | 52 | WEHAKDWPT | 105 |
| HSA-49 | EWYQTRWPG | 53 | FHSKVLDKA | 106 |
| HSA-50 | EFWQRHWPG | 54 | YGAQKQAVW | 107 |
| HSA-51 | KFYERHWPG | 55 | FSASHFTSQ | 108 |
| Con- | GWWQRRWPG | 56 | X₁X₂AX₃K | 109 |
| sensus | | | X₄DX₅Q | |

In some embodiments, (Xaa)ₙ comprises an amino acid sequence having at least 80% or at least 90% identity to the amino acid sequence of any one of SEQ ID NOS: 6-55, 134-135. In some embodiments, (Xaa)ₙ comprises an amino acid sequence having 80% to 90% identity to the amino acid sequence of any one of SEQ ID NOS: 6-55, 134-135. In some embodiments, (Xaa)ₙ comprises the amino acid sequence of any one of SEQ ID NOS: 6-55, 134-135.

In some embodiments, (Xaa)ₙ comprises an amino acid sequence having at least 80% or at least 90% identity to the amino acid sequence of any one of SEQ ID NOS: 22, 24, 26, 35, 40, 41, and 45. In some embodiments, (Xaa)ₙ comprises an amino acid sequence having at least 80% identity to the amino acid sequence of SEQ ID NO: 22. In some embodiments, (Xaa)ₙ comprises an amino acid sequence having at least 80% identity to the amino acid sequence of SEQ ID NO: 24. In some embodiments, (Xaa)ₙ comprises an amino acid sequence having at least 80% identity to the amino acid sequence of SEQ ID NO: 26. In some embodiments, (Xaa)ₙ comprises an amino acid sequence having at least 80% identity to the amino acid sequence of SEQ ID NO: 35. In some embodiments, (Xaa)ₙ comprises an amino acid sequence having at least 80% identity to the amino acid sequence of SEQ ID NO: 40. In some embodiments, (Xaa)ₙ comprises an amino acid sequence having at least 80% identity to the amino acid sequence of SEQ ID NO: 41. In some embodiments, (Xaa)ₙ comprises an amino acid sequence having at least 80% identity to the amino acid sequence of SEQ ID NO: 45. In some embodiments, (Xaa)ₙ comprises an amino acid sequence having at least 90% identity to the amino acid sequence of SEQ ID NO: 22. In some embodiments, (Xaa)ₙ comprises an amino acid sequence having at least 90% identity to the amino acid sequence of SEQ ID NO: 24. In some embodiments, (Xaa)ₙ comprises an amino acid sequence having at least 90% identity to the amino acid sequence of SEQ ID NO: 26. In some embodiments, (Xaa)ₙ comprises an amino acid sequence having at least 90% identity to the amino acid sequence of SEQ ID NO: 35. In some embodiments, (Xaa)ₙ comprises an amino acid sequence having at least 90% identity to the amino acid sequence of SEQ ID NO: 40. In some embodiments, (Xaa)ₙ comprises an amino acid sequence having at least 90% identity to the amino acid sequence of SEQ ID NO: 41. In some embodiments, (Xaa)ₙ comprises an amino acid sequence having at least 90% identity to the amino acid sequence of SEQ ID NO: 45.

In some embodiments, (Xaa)ₙ comprises an amino acid sequence having 80% to 90% identity to the amino acid sequence of any one of SEQ ID NOS: 22, 24, 26, 35, 40, 41, and 45. In some embodiments, (Xaa)ₙ comprises an amino acid sequence having 80% to 90% identity to the amino acid sequence of SEQ ID NO: 22. In some embodiments, (Xaa)ₙ comprises an amino acid sequence having 80% to 90% identity to the amino acid sequence of SEQ ID NO: 24. In some embodiments, (Xaa)ₙ comprises an amino acid sequence having 80% to 90% identity to the amino acid sequence of SEQ ID NO: 26. In some embodiments, (Xaa)ₙ comprises an amino acid sequence having 80% to 90% identity to the amino acid sequence of SEQ ID NO: 35. In some embodiments, (Xaa)ₙ comprises an amino acid sequence having 80% to 90% identity to the amino acid sequence of SEQ ID NO: 40. In some embodiments, (Xaa)ₙ comprises an amino acid sequence having 80% to 90% identity to the amino acid sequence of SEQ ID NO: 41. In some embodiments, (Xaa)ₙ comprises an amino acid sequence having 80% to 90% identity to the amino acid sequence of SEQ ID NO: 45.

In some embodiments, (Xaa)ₙ comprises the amino acid sequence of any one of SEQ ID NOS: 22, 24, 26, 35, 40, 41, and 45. In some embodiments, (Xaa)ₙ comprises the amino acid sequence of SEQ ID NO: 22. In some embodiments, (Xaa)ₙ comprises the amino acid sequence of SEQ ID NO: 24. In some embodiments, (Xaa)ₙ comprises the amino acid sequence of SEQ ID NO: 26. In some embodiments, (Xaa)ₙ comprises the amino acid sequence of SEQ ID NO: 35. In some embodiments, (Xaa)ₙ comprises the amino acid sequence of SEQ ID NO: 40. In some embodiments, (Xaa)ₙ comprises the amino acid sequence of SEQ ID NO: 41. In some embodiments, (Xaa)ₙ comprises the amino acid sequence of SEQ ID NO: 45.

In some embodiments, (Xaa)ₘ comprises an amino acid sequence having at least 80% or at least 90% identity to the amino acid sequence of any one of SEQ ID NOS: 57-108. In some embodiments, (Xaa)ₘ comprises an amino acid sequence having 80% to 90% identity to the amino acid sequence of any one of SEQ ID NOS: 57-108. In some embodiments, (Xaa)ₘ comprises the amino acid sequence of any one of SEQ ID NOS: 57-108.

In some embodiments, (Xaa)ₘ comprises an amino acid sequence having at least 80% or at least 90% identity to the amino acid sequence of any one of SEQ ID NOS: 75, 77, 79, 88, 93, 94, and 98. In some embodiments, (Xaa)ₘ comprises an amino acid sequence having at least 80% identity to the amino acid sequence of SEQ ID NO: 75. In some embodiments, (Xaa)ₘ comprises an amino acid sequence having at least 80% identity to the amino acid sequence of SEQ ID NO: 77. In some embodiments, (Xaa)ₘ comprises an amino acid sequence having at least 80% identity to the amino acid sequence of SEQ ID NO: 79. In some embodiments, (Xaa)ₘ comprises an amino acid sequence having at least 80% identity to the amino acid sequence of SEQ ID NO: 88. In some embodiments, (Xaa)ₘ comprises an amino acid sequence having at least 80% identity to the amino acid sequence of SEQ ID NO: 93. In some embodiments, (Xaa)ₘ comprises an amino acid sequence having at least 80% identity to the amino acid sequence of SEQ ID NO: 94. In some embodiments, (Xaa)ₘ comprises an amino acid sequence having at least 80% identity to the amino acid sequence of SEQ ID NO: 98. In some embodiments, (Xaa)m comprises an amino acid sequence having at least 90% identity to the amino acid sequence of SEQ ID NO: 75. In some embodiments, (Xaa)ₘ comprises an amino acid sequence having at least 90% identity to the amino acid sequence of SEQ ID NO: 77. In some embodiments, (Xaa)ₘ comprises an amino acid sequence having at least 90% identity to the amino acid sequence of SEQ ID NO: 79. In some embodiments, (Xaa)ₘ comprises an amino acid sequence having at least 90% identity to the amino acid sequence of SEQ ID NO: 88. In some embodiments, (Xaa)ₘ comprises an amino acid sequence having at least 90% identity to the amino acid sequence of SEQ ID NO: 93. In some embodiments, (Xaa)ₘ comprises an amino acid sequence having at least 90% identity to the amino acid sequence of SEQ ID NO: 94. In some embodiments, (Xaa)ₘ comprises an amino acid sequence having at least 90% identity to the amino acid sequence of SEQ ID NO: 98.

In some embodiments, (Xaa)ₘ comprises an amino acid sequence having 80% to 90% identity to the amino acid sequence of any one of SEQ ID NOS: 75, 77, 79, 88, 93, 94, and 98. In some embodiments, (Xaa)ₘ comprises the amino acid sequence of any one of SEQ ID NOS: 75, 77, 79, 88, 93, 94, and 98. In some embodiments, (Xaa)ₘ comprises an amino acid sequence having 80% to 90% identity to the amino acid sequence of SEQ ID NO: 75. In some embodiments, (Xaa)ₘ comprises an amino acid sequence having 80% to 90% identity to the amino acid sequence of SEQ ID NO: 77. In some embodiments, (Xaa)ₘ comprises an amino acid sequence having 80% to 90% identity to the amino acid sequence of SEQ ID NO: 79. In some embodiments, (Xaa)ₘ comprises an amino acid sequence having 80% to 90% identity to the amino acid sequence of SEQ ID NO: 88. In some embodiments, (Xaa)ₘ comprises an amino acid sequence having 80% to 90% identity to the amino acid sequence of SEQ ID NO: 93. In some embodiments, (Xaa)ₘ comprises an amino acid sequence having 80% to 90% identity to the amino acid sequence of SEQ ID NO: 94. In some embodiments, (Xaa)ₘ comprises an amino acid sequence having 80% to 90% identity to the amino acid sequence of SEQ ID NO: 98.

In some embodiments, an anti-HSA AFFIMER® polypeptide comprises an amino acid sequence selected from any one of SEQ ID NOS: 110-116 and 133 (Table 2).

TABLE 2
Examples of HSA AFFIMER ® Polypeptide Sequences

| | | SEQ ID |
|---|---|---|
| Name | Sequence | NO: |
| HSA-18 | MIPRGLSEAKPATPEIQEIVDKVKPQLEEKTNETYGKLEAVQYKTQVLA | 110 |
| | DWWQAKWPHSTNYYIKVRAGDNKYMHLKVFNGPYKVHQSSGGADRVLTG | |
| | YQVDKNKDDELTGF | |
| HSA-20 | MIPRGLSEAKPATPEIQEIVDKVKPQLEEKTNETYGKLEAVQYKTQVLA | 111 |
| | GYWAAKWPGSTNYYIKVRAGDNKYMHLKVFNGPFPNTSYDLQADRVLTG | |
| | YQVDKNKDDELTGF | |
| HSA-22 | MIPRGLSEAKPATPEIQEIVDKVKPQLEEKTNETYGKLEAVQYKTQVLA | 112 |
| | NWYQQRWPGSTNYYIKVRAGDNKYMHLKVFNGPWHNYGESSGADRVLTG | |
| | YQVDKNKDDELTGF | |
| HSA-31 | MIPRGLSEAKPATPEIQEIVDKVKPQLEEKTNETYGKLEAVQYKTQVLA | 113 |
| | VWGPEYQHQSTNYYIKVRAGDNKYMHLKVFNGPNAGWPLVPEADRVLTG | |
| | YQVDKNKDDELTGF | |
| HSA-36 | MIPRGLSEAKPATPEIQEIVDKVKPQLEEKTNETYGKLEAVQYKTQVLA | 114 |
| | GHYARYWPGSTNYYIKVRAGDNKYMHLKVFNGPWAQKSKVHQADRVLTG | |
| | YQVDKNKDDELTGF | |
| HSA-37 | MIPRGLSEAKPATPEIQEIVDKVKPQLEEKTNETYGKLEAVQYKTQVLA | 115 |
| | GFWASKWPGSTNYYIKVRAGDNKYMHLKVFNGPFTAVSKKDAADRVLTG | |
| | YQVDKNKDDELTGF | |
| HSA-41 | MIPRGLSEAKPATPEIQEIVDKVKPQLEEKTNETYGKLEAVQYKTQVLA | 116 |
| | NFFQRRWPGSTNYYIKVRAGDNKYMHLKVFNGPWKFRNTDRGADRVLTG | |
| | YQVDKNKDDELTGF | |
| HSA-52 | MIPRGLSEAKPATPEIQEIVDKVKPQLEEKTNETYGKLEAVQYKTQVLA | 133 |
| | NFFQRRWPGSTNYYIKVRAGDNKYMHLKVFNGPWKFRNTDRGADRVLTG | |
| | YQVDKNKDDELTGFAAAGGRAEQKLISEEDLGCAENLYFQGGAAGHHHH | |
| | HH | |

In some embodiments, an anti-HSA AFFIMER® polypeptide comprises an amino acid sequence having at least 80% or at least 90% identity to the amino acid sequence of any one of SEQ ID NOS: 110-116 and 133. In some embodiments, an anti-HSA AFFIMER® polypeptide comprises an amino acid sequence having at least 80% identity to the amino acid sequence of SEQ ID NO: 110. In some embodiments, an anti-HSA AFFIMER® polypeptide comprises an amino acid sequence having at least 80% identity to the amino acid sequence of SEQ ID NO: 111. In some embodiments, an anti-HSA AFFIMER® polypeptide comprises an amino acid sequence having at least 80% identity to the amino acid sequence of SEQ ID NO: 112. In some embodiments, an anti-HSA AFFIMER® polypeptide comprises an amino acid sequence having at least 80% identity to the amino acid sequence of SEQ ID NO: 113. In some embodiments, an anti-HSA AFFIMER® polypeptide comprises an amino acid sequence having at least 80% identity to the amino acid sequence of SEQ ID NO: 114. In some embodiments, an anti-HSA AFFIMER® polypeptide comprises an amino acid sequence having at least 80% identity to the amino acid sequence of SEQ ID NO: 115. In some embodiments, an anti-HSA AFFIMER® polypeptide comprises an amino acid sequence having at least 80% identity to the amino acid sequence of SEQ ID NO: 116. In some embodiments, an anti-HSA AFFIMER® polypeptide comprises an amino acid sequence having at least 80% identity to the amino acid sequence of SEQ ID NO: 133.

In some embodiments, an anti-HSA AFFIMER® polypeptide comprises an amino acid sequence having at least 90% identity to the amino acid sequence of SEQ ID NO: 110. In some embodiments, an anti-HSA AFFIMER® polypeptide comprises an amino acid sequence having at least 90% identity to the amino acid sequence of SEQ ID NO: 111. In some embodiments, an anti-HSA AFFIMER® polypeptide comprises an amino acid sequence having at least 90% identity to the amino acid sequence of SEQ ID NO: 112. In some embodiments, an anti-HSA AFFIMER® polypeptide comprises an amino acid sequence having at least 90% identity to the amino acid sequence of SEQ ID NO: 113. In some embodiments, an anti-HSA AFFIMER® polypeptide comprises an amino acid sequence having at least 90% identity to the amino acid sequence of SEQ ID NO: 114. In some embodiments, an anti-HSA AFFIMER® polypeptide comprises an amino acid sequence having at least 90% identity to the amino acid sequence of SEQ ID NO: 115. In some embodiments, an anti-HSA AFFIMER® polypeptide comprises an amino acid sequence having at least 90% identity to the amino acid sequence of SEQ ID NO: 116. In some embodiments, an anti-HSA AFFIMER® polypeptide comprises an amino acid sequence having at least 90% identity to the amino acid sequence of SEQ ID NO: 133.

In some embodiments, an anti-HSA AFFIMER® polypeptide comprises an amino acid sequence having 80% to 90% identity to the amino acid sequence of any one of SEQ ID NOS: 110-116 and 133. In some embodiments, an anti-HSA AFFIMER® polypeptide comprises the amino acid sequence of any one of SEQ ID NOS: 110-116 and 133. In some embodiments, an anti-HSA AFFIMER® polypeptide comprises an amino acid sequence having 80% to 90% identity to the amino acid sequence of SEQ ID NO: 110. In some embodiments, an anti-HSA AFFIMER® polypeptide comprises an amino acid sequence having 80% to 90% identity to the amino acid sequence of SEQ ID NO: 111. In some embodiments, an anti-HSA AFFIMER® polypeptide comprises an amino acid sequence having 80% to 90% identity to the amino acid sequence of SEQ ID NO: 112. In some embodiments, an anti-HSA AFFIMER® polypeptide comprises an amino acid sequence having 80% to 90% identity to the amino acid sequence of SEQ ID NO: 113. In some embodiments, an anti-HSA AFFIMER® polypeptide comprises an amino acid sequence having 80% to 90% identity to the amino acid sequence of SEQ ID NO: 114. In some embodiments, an anti-HSA AFFIMER® polypeptide comprises an amino acid sequence having 80% to 90% identity to the amino acid sequence of SEQ ID NO: 115. In some embodiments, an anti-HSA AFFIMER® polypeptide comprises an amino acid sequence having 80% to 90% identity to the amino acid sequence of SEQ ID NO: 116. In some embodiments, an anti-HSA AFFIMER® polypeptide comprises an amino acid sequence having 80% to 90% identity to the amino acid sequence of SEQ ID NO: 133.

An anti-HSA AFFIMER® polypeptide provided herein, in some embodiments, is linked to another molecule and extend the half-life of that molecule (e.g., a therapeutic polypeptide). Provided herein is a range of anti-HSA AFFIMERS®, with a range of binding affinities, for example, that cross-react with other species such as mouse and cynomolgous (cyno) monkey. These anti-HSA AFFIMERS®, in some embodiments, make up what is referred to as the AFFIMER XT™ platform. These anti-HSA AFFIMERS® have been shown in in vivo pharmacokinetic (PK) studies to extend, in a controlled manner, the serum half-life of any other AFFIMER® polypeptide therapeutic to which it is conjugated in a single genetic fusion, for example, that can be made in E. Coli. AFFIMER XT™ can also be used to extend the half-life of other peptide or protein therapeutics.

The term half-life refers to the amount of time it takes for a substance, such as a therapeutic AFFIMER® polypeptide, to lose half of its pharmacologic or physiologic activity or concentration. Biological half-life can be affected by elimination, excretion, degradation (e.g., enzymatic degradation) of the substance, or absorption and concentration in certain organs or tissues of the body. Biological half-life can be assessed, for example, by determining the time it takes for the blood plasma concentration of the substance to reach half its steady state level (“plasma half-life”).

In some embodiments, an anti-HSA AFFIMER® polypeptide extends the serum half-life of a molecule (e.g., a therapeutic polypeptide) in vivo. For example, an anti-HSA AFFIMER® polypeptide may extend the half-life of a molecule by at least 2-fold, relative to the half-life of the molecule not linked to an anti-HSA AFFIMER® polypeptide. In some embodiments, an anti-HSA AFFIMER® polypeptide extends the half-life of a molecule by at least 3-fold, at least 4-fold, at least 5-fold, at least 6-fold, at least 7-fold, at least 8-fold, at least 9-fold, at least 10-fold, at least 20-fold, or at least 30-fold, relative to the half-life of the molecule not linked to an anti-HSA AFFIMER® polypeptide. In some embodiments, an anti-HSA AFFIMER® polypeptide extends the half-life of a molecule by 2-fold to 5-fold, 2-fold to 10-fold, 3-fold to 5-fold, 3-fold to 10-fold, 15-fold to 5-fold, 4-fold to 10-fold, or 5-fold to 10-fold, relative to the half-life of the molecule not linked to an anti-HSA AFFIMER® polypeptide. In some embodiments, an anti-HSA AFFIMER® polypeptide extends the half-life of a molecule by at least 6 hours, at least 12 hours, at least 24 hours, at least 48 hours, at least 72 hours, at least 96 hours, for example, at least 1 week after in vivo administration, relative to the half-life of the molecule not linked to an anti-HSA AFFIMER® polypeptide.

In some embodiments, an anti-HSA AFFIMER® polypeptide has an extended serum half-life and comprises an amino acid sequence selected from any one of SEQ ID NOS: 117-127 (Table 3).

TABLE 3
Examples of half-life extension in-line fusion
AFFIMER ® Polypeptide sequences

| | SEQ |
|---|---|
| | ID |
| Sequence | NO: |
| MIPRGLSEAKPATPEIQEIVDKVKPQLEEKTNETYGKLEAVQYKT | 117 |
| QVLAKEHGPDSWWSTNYYIKVRAGDNKYMHLKVFNGPQEKNQWVE | |
| EADRVLTGYQVDKNKDDELTGFGGGGSGGGGSGGGGSGGGGSGGG | |
| GSGGGGSMIPRGLSEAKPATPEIQEIVDKVKPQLEEKTNETYGKL | |
| EAVQYKTQVLANFFQRRWPGSTNYYIKVRAGDNKYMHLKVENGPW | |
| KFRNTDRGADRVLTGYQVDKNKDDELTGF | |
| MIPRGLSEAKPATPEIQEIVDKVKPQLEEKTNETYGKLEAVQYKT | 118 |
| QVLAKEHGPDSWWSTNYYIKVRAGDNKYMHLKVFNGPQEKNQWVE | |
| EADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLANFFQRRWPGSTNYYIKVRAGDNKYMHLKVFNGP | |
| WKFRNTDRGADRVLTGYQVDKNKDDELTGF | |
| MIPRGLSEAKPATPEIQEIVDKVKPQLEEKTNETYGKLEAVQYKT | 119 |
| QVLAHAYGPRDWDSTNYYIKVRAGDNKYMHLKVFNGPPADHVLEE | |
| AADRVLTGYQVDKNKDDELTGFGGGGSGGGGSGGGGSGGGGSGGG | |
| GSGGGGSMIPRGLSEAKPATPEIQEIVDKVKPQLEEKTNETYGKL | |
| EAVQYKTQVLANFFQRRWPGSTNYYIKVRAGDNKYMHLKVFNGPW | |
| KFRNTDRGADRVLTGYQVDKNKDDELTGF | |
| MIPRGLSEAKPATPEIQEIVDKVKPQLEEKTNETYGKLEAVQYKT | 120 |
| QVLAHAYGPRDWDSTNYYIKVRAGDNKYMHLKVFNGPPADHVLEE | |
| AADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLANFFQRRWPGSTNYYIKVRAGDNKYMHLKVFNGP | |
| WKFRNTDRGADRVLTGYQVDKNKDDELTGF | |
| MIPRGLSEAKPATPEIQEIVDKVKPQLEEKTNETYGKLEAVQYKT | 121 |
| QVLAKEYGPEEWWSTNYYIKVRAGDNKYMHLKVFNGPGDYEQVLI | |
| HADRVLTGYQVDKNKDDELTGFGGGGSGGGGSGGGGSGGGGSGGG | |
| GSGGGGSMIPRGLSEAKPATPEIQEIVDKVKPQLEEKTNETYGKL | |
| EAVQYKTQVLANFFQRRWPGSTNYYIKVRAGDNKYMHLKVFNGPW | |
| KFRNTDRGADRVLTGYQVDKNKDDELTGF | |
| MIPRGLSEAKPATPEIQEIVDKVKPQLEEKTNETYGKLEAVQYKT | 122 |
| QVLAKEYGPEEWWSTNYYIKVRAGDNKYMHLKVFNGPGDYEQVLI | |
| HADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLANFFQRRWPGSTNYYIKVRAGDNKYMHLKVFNGP | |
| WKFRNTDRGADRVLTGYQVDKNKDDELTGF | |
| MIPRGLSEAKPATPEIQEIVDKVKPQLEEKTNETYGKLEAVQYKT | 123 |
| QVLAREGRQDWVLSTNYYIKVRAGDNKYMHLKVFNGPWVPFPHQQ | |
| LADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAREGRQDWVLSTNYYIKVRAGDNKYMHLKVFNGP | |
| WVPFPHQQLADRVLTGYQVDKNKDDELTGFAEAAAKEAAAKEAAA | |
| KEAAAKEAAAKEAAAKMIPRGLSEAKPATPEIQEIVDKVKPQLEE | |
| KTNETYGKLEAVQYKTQVLANFFQRRWPGSTNYYIKVRAGDNKYM | |
| HLKVFNGPWKFRNTDRGADRVLTGYQVDKNKDDELTGF | |
| MIPRGLSEAKPATPEIQEIVDKVKPQLEEKTNETYGKLEAVQYKT | 124 |
| QVLAREGRQDWVLSTNYYIKVRAGDNKYMHLKVFNGPWVPFPHQQ | |
| LADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLANFFQRRWPGSTNYYIKVRAGDNKYMHLKVFNGP | |
| WKFRNTDRGADRVLTGYQVDKNKDDELTGFAEAAAKEAAAKEAAA | |
| KEAAAKEAAAKEAAAKMIPRGLSEAKPATPEIQEIVDKVKPQLEE | |
| KTNETYGKLEAVQYKTQVLAREGRQDWVLSTNYYIKVRAGDNKYM | |
| HLKVFNGPWVPFPHQQLADRVLTGYQVDKNKDDELTGF | |
| MIPRGLSEAKPATPEIQEIVDKVKPQLEEKTNETYGKLEAVQYKT | 125 |
| QVLANFFQRRWPGSTNYYIKVRAGDNKYMHLKVFNGPWKFRNTDR | |
| GADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAREGRQDWVLSTNYYIKVRAGDNKYMHLKVFNGP | |
| WVPFPHQQLADRVLTGYQVDKNKDDELTGFAEAAAKEAAAKEAAA | |
| KEAAAKEAAAKEAAAKMIPRGLSEAKPATPEIQEIVDKVKPQLEE | |
| KTNETYGKLEAVQYKTQVLAREGRQDWVLSTNYYIKVRAGDNKYM | |
| HLKVFNGPWVPFPHQQLADRVLTGYQVDKNKDDELTGF | |
| MIPRGLSEAKPATPEIQEIVDKVKPQLEEKTNETYGKLEAVQYKT | 126 |
| QVLAREGRQDWVLSTNYYIKVRAGDNKYMHLKVFNGPWVPFPHQQ | |
| LADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAREGRQDWVLSTNYYIKVRAGDNKYMHLKVFNGP | |
| WVPFPHQQLADRVLTGYQVDKNKDDELTGFAEAAAKEAAAKEAAA | |
| KEAAAKEAAAKEAAAKMIPRGLSEAKPATPEIQEIVDKVKPQLEE | |
| KTNETYGKLEAVQYKTQVLANFFQRRWPGSTNYYIKVRAGDNKYM | |
| HLKVFNGPWKFRNTDRGADRVLTGYQVDKNKDDELTGFC | |
| MIPRGLSEAKPATPEIQEIVDKVKPQLEEKTNETYGKLEAVQYKT | 127 |
| QVLAFALPEFEYMSTNYYIKVRAGDNKYMHLKVFNGPPMIRRKNE | |
| VADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAFALPEFEYMSTNYYIKVRAGDNKYMHLKVFNGP | |
| PMIRRKNEVADRVLTGYQVDKNKDDELTGFAEAAAKEAAAKEAAA | |
| KEAAAKEAAAKEAAAKMIPRGLSEAKPATPEIQEIVDKVKPQLEE | |
| KTNETYGKLEAVQYKTQVLANFFQRRWPGSTNYYIKVRAGDNKYM | |
| HLKVFNGPWKFRNTDRGADRVLTGYQVDKNKDDELTGF | |
| MIPRGLSEAKPATPEIQEIVDKVKPQLEEKTNETYGKLEAVQYKT | 128 |
| QVLAGGGGGGGGGSTNYYIKVRAGDNKYMHLKVFNGPGGGGGGGG | |
| GADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAGGGGGGGGGSTNYYIKVRAGDNKYMHLKVFNGP | |
| GGGGGGGGGADRVLTGYQVDKNKDDELTGFAEAAAKEAAAKEAAA | |
| KEAAAKEAAAKEAAAKMIPRGLSEAKPATPEIQEIVDKVKPQLEE | |
| KTNETYGKLEAVQYKTQVLANFFQRRWPGSTNYYIKVRAGDNKYM | |
| HLKVFNGPWKFRNTDRGADRVLTGYQVDKNKDDELTGF | |
| MIPRGLSEAKPATPEIQEIVDKVKPQLEEKTNETYGKLEAVQYKT | 129 |
| QVLAREGRQDWVLSTNYYIKVRAGDNKYMHLKVFNGPWVPFPHQQ | |
| LADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAREGRQDWVLSTNYYIKVRAGDNKYMHLKVFNGP | |
| WVPFPHQQLADRVLTGYQVDKNKDDELTGF | |
| MIPRGLSEAKPATPEIQEIVDKVKPQLEEKTNETYGKLEAVQYKT | 130 |
| QVLAREGRQDWVLSTNYYIKVRAGDNKYMHLKVFNGPWVPFPHQQ | |
| LADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLAREGRQDWVLSTNYYIKVRAGDNKYMHLKVFNGP | |
| WVPFPHQQLADRVLTGYQVDKNKDDELTGFC | |
| MIPRGLSEAKPATPEIQEIVDKVKPQLEEKTNETYGKLEAVQYKT | 131 |
| QVLANFFQRRWPGSTNYYIKVRAGDNKYMHLKVFNGPWKFRNTDR | |
| GADRVLTGYQVDKNKDDELTGFAEAAAKEAAAKEAAAKEAAAKEA | |
| AAKEAAAKMIPRGLSEAKPATPEIQEIVDKVKPQLEEKTNETYGK | |
| LEAVQYKTQVLANFFQRRWPGSTNYYIKVRAGDNKYMHLKVFNGP | |
| WKFRNTDRGADRVLTGYQVDKNKDDELTGF | |
| MIPRGLSEAKPATPEIQEIVDKVKPQLEEKTNETYGKLEAVQYKT | 132 |
| QVLANFFQRRWPGSTNYYIKVRAGDNKYMHLKVFNGPWKFRNTDR | |
| GADRVLTGYQVDKNKDDELTGFGGGGSGGGGSGGGGSGGGGSGGG | |
| GSGGGGSMIPRGLSEAKPATPEIQEIVDKVKPQLEEKTNETYGKL | |
| EAVQYKTQVLANFFQRRWPGSTNYYIKVRAGDNKYMHLKVFNGPW | |
| KFRNTDRGADRVLTGYQVDKNKDDELTGF | |

In some embodiments, an anti-HSA AFFIMER® polypeptide comprises an amino acid sequence having at least 80% or at least 90% identity to the amino acid sequence of any one of SEQ ID NOS: 117-127. In some embodiments, an anti-HSA AFFIMER® polypeptide comprises an amino acid sequence having at least 80% identity to the amino acid sequence of SEQ ID NO: 117. In some embodiments, an anti-HSA AFFIMER® polypeptide comprises an amino acid sequence having at least 80% identity to the amino acid sequence of SEQ ID NO: 118. In some embodiments, an anti-HSA AFFIMER® polypeptide comprises an amino acid sequence having at least 80% identity to the amino acid sequence of SEQ ID NO: 119. In some embodiments, an anti-HSA AFFIMER® polypeptide comprises an amino acid sequence having at least 80% identity to the amino acid sequence of SEQ ID NO: 120. In some embodiments, an anti-HSA AFFIMER® polypeptide comprises an amino acid sequence having at least 80% identity to the amino acid sequence of SEQ ID NO: 121. In some embodiments, an anti-HSA AFFIMER® polypeptide comprises an amino acid sequence having at least 80% identity to the amino acid sequence of SEQ ID NO: 122. In some embodiments, an anti-HSA AFFIMER® polypeptide comprises an amino acid sequence having at least 80% identity to the amino acid sequence of SEQ ID NO: 123. In some embodiments, an anti-HSA AFFIMER® polypeptide comprises an amino acid sequence having at least 80% identity to the amino acid sequence of SEQ ID NO: 124. In some embodiments, an anti-HSA AFFIMER® polypeptide comprises an amino acid sequence having at least 80% identity to the amino acid sequence of SEQ ID NO: 125. In some embodiments, an anti-HSA AFFIMER® polypeptide comprises an amino acid sequence having at least 80% identity to the amino acid sequence of SEQ ID NO: 126. In some embodiments, an anti-HSA AFFIMER® polypeptide comprises an amino acid sequence having at least 80% identity to the amino acid sequence of SEQ ID NO: 127.

In some embodiments, an anti-HSA AFFIMER® polypeptide comprises an amino acid sequence having at least 90% identity to the amino acid sequence of SEQ ID NO: 117. In some embodiments, an anti-HSA AFFIMER® polypeptide comprises an amino acid sequence having at least 90% identity to the amino acid sequence of SEQ ID NO: 118. In some embodiments, an anti-HSA AFFIMER® polypeptide comprises an amino acid sequence having at least 90% identity to the amino acid sequence of SEQ ID NO: 119. In some embodiments, an anti-HSA AFFIMER® polypeptide comprises an amino acid sequence having at least 90% identity to the amino acid sequence of SEQ ID NO: 120. In some embodiments, an anti-HSA AFFIMER® polypeptide comprises an amino acid sequence having at least 90% identity to the amino acid sequence of SEQ ID NO: 121. In some embodiments, an anti-HSA AFFIMER® polypeptide comprises an amino acid sequence having at least 90% identity to the amino acid sequence of SEQ ID NO: 122. In some embodiments, an anti-HSA AFFIMER® polypeptide comprises an amino acid sequence having at least 90% identity to the amino acid sequence of SEQ ID NO: 123. In some embodiments, an anti-HSA AFFIMER® polypeptide comprises an amino acid sequence having at least 90% identity to the amino acid sequence of SEQ ID NO: 124. In some embodiments, an anti-HSA AFFIMER® polypeptide comprises an amino acid sequence having at least 90% identity to the amino acid sequence of SEQ ID NO: 125. In some embodiments, an anti-HSA AFFIMER® polypeptide comprises an amino acid sequence having at least 90% identity to the amino acid sequence of SEQ ID NO: 126. In some embodiments, an anti-HSA AFFIMER® polypeptide comprises an amino acid sequence having at least 90% identity to the amino acid sequence of SEQ ID NO: 127.

In some embodiments, an anti-HSA AFFIMER® polypeptide comprises an amino acid sequence having 80% to 90% identity to the amino acid sequence of any one of SEQ ID NOS: 117-127. In some embodiments, an anti-HSA AFFIMER® polypeptide comprises the amino acid sequence of any one of SEQ ID NOS: 117-127. In some embodiments, an anti-HSA AFFIMER® polypeptide comprises an amino acid sequence having 80% to 90% identity to the amino acid sequence of SEQ ID NO: 117. In some embodiments, an anti-HSA AFFIMER® polypeptide comprises an amino acid sequence having 80% to 90% identity to the amino acid sequence of SEQ ID NO: 118. In some embodiments, an anti-HSA AFFIMER® polypeptide comprises an amino acid sequence having 80% to 90% identity to the amino acid sequence of SEQ ID NO: 119. In some embodiments, an anti-HSA AFFIMER® polypeptide comprises an amino acid sequence having 80% to 90% identity to the amino acid sequence of SEQ ID NO: 120. In some embodiments, an anti-HSA AFFIMER® polypeptide comprises an amino acid sequence having 80% to 90% identity to the amino acid sequence of SEQ ID NO: 121. In some embodiments, an anti-HSA AFFIMER® polypeptide comprises an amino acid sequence having 80% to 90% identity to the amino acid sequence of SEQ ID NO: 122. In some embodiments, an anti-HSA AFFIMER® polypeptide comprises an amino acid sequence having 80% to 90% identity to the amino acid sequence of SEQ ID NO: 123. In some embodiments, an anti-HSA AFFIMER® polypeptide comprises an amino acid sequence having 80% to 90% identity to the amino acid sequence of SEQ ID NO: 124. In some embodiments, an anti-HSA AFFIMER® polypeptide comprises an amino acid sequence having 80% to 90% identity to the amino acid sequence of SEQ ID NO: 125. In some embodiments, an anti-HSA AFFIMER® polypeptide comprises an amino acid sequence having 80% to 90% identity to the amino acid sequence of SEQ ID NO: 126. In some embodiments, an anti-HSA AFFIMER® polypeptide comprises an amino acid sequence having 80% to 90% identity to the amino acid sequence of SEQ ID NO: 127.

Fc Domains

In some embodiments, the half-life extension moiety comprises an antibody Fc domain. The term “Fc domain” includes functional fragments of antibody Fc domains. A therapeutic conjugate may comprise, for example, the Fc region of an antibody (which facilitates effector functions and pharmacokinetics) linked to a drug moiety through an enzyme-cleavable linker (also referred to herein as an “Fc fusion”). In some embodiments, Fc fusions can be dimerized to form Fc fusion homodimers, or using non-identical Fc domains, to form Fc fusion heterodimers.

There are several reasons for choosing the Fc domain of human antibodies for use in generating the therapeutic conjugates of the present disclosure. The principle rationale is to produce a stable protein, large enough to demonstrate a similar pharmacokinetic profile compared with those of antibodies, and to take advantage of the properties imparted by the Fc domain; this includes the salvage neonatal FcRn receptor pathway involving FcRn-mediated recycling of the Fc fusion to the cell surface post endocytosis, avoiding lysosomal degradation and resulting in release back into the bloodstream, thus contributing to an extended serum half-life. Another obvious advantage is the Fc domain's binding to Protein A, which can simplify downstream processing during production of the therapeutic conjugates and permit generation of highly pure preparation of the therapeutic conjugates.

In some embodiments, an Fc domain includes the constant region of an antibody excluding the first constant region immunoglobulin domain. Thus, Fc domain refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Oy2 and Cy3 and the hinge between Oy1 and Oy2. Although the boundaries of the Fc domain may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, NIH, Bethesda, Md. (1991)). Fc may refer to this region in isolation, or this region in the context of a whole antibody, antibody fragment, or Fc fusion protein. Polymorphisms have been observed at a number of different Fc positions and are also included as Fc domains as used herein.

An Fc domain includes functional fragments of antibody Fc domains. A functional Fc fragment retains the ability to bind FcRn. A functional Fc fragment binds to FcRn but does not possess effector function. The ability of the Fc region or fragment thereof to bind to FcRn can be determined by standard binding assays known in the art. Exemplary effector functions include Clq binding; complement dependent cytotoxicity (CDC); Fc receptor binding; antibody-dependent cell-mediated cytotoxicity (ADCC); phagocytosis; and down regulation of cell surface receptors (e.g., B cell receptor; BCR). Such effector functions can be assessed using various assays known in the art for evaluating such antibody effector functions.

In an exemplary embodiment, the Fc domain is derived from an IgG1 subclass, however, other subclasses (e.g., IgG2, IgG3, and IgG4) may also be used. In some embodiments, the Fc domain is derived from an IgG1 subclass and further comprises a LALA mutation.

Other non-limiting examples of Fc domains, functional Fc fragments and particular sequences are provided in WO 2019/236567, incorporated herein by reference.

### Biocompatible Polymers

A wide variety of biocompatible macromolecular polymers and other molecules can be used as a half-life extension moiety. The molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

For the purpose of half-life extension, various methods including pegylation, polysialylation, HESylation, glycosylation, or recombinant PEG analogue fused to flexible and hydrophilic amino acid chain (500 to 600 amino acids) have been developed (See Chapman, (2002) Adv Drug Deliv Rev. 54. 531-545; Schlapschy et al., (2007) Prot Eng Des Sel. 20, 273-283; Contermann (2011) Curr Op Biotechnol. 22, 868-876; Jevsevar et al., (2012) Methods Mol Biol. 901, 233-246).

Examples of biocompatible polymers that may be used as half-life extension moieties include but are not limited to polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethylene glycol or PEG); discrete PEG (dPEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof; hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof; terpolymers thereof; mixtures thereof; and derivatives of the foregoing.

The polymer selected may be water soluble so that the therapeutic conjugate of which it is a component does not precipitate in an aqueous environment, such as a physiological environment. The water-soluble polymer may be any structural form including but not limited to linear, forked or branched. In some embodiments, the water soluble polymer is a poly(alkylene glycol), such as poly(ethylene glycol) (PEG), but other water soluble polymers can also be used. By way of example, PEG is used in some embodiments of this disclosure, provided the properties of the particular PEG selected extends the circulating serum half-life of the therapeutic conjugate in accordance with the present disclosure.

In some embodiments, the biocompatible polymer is selected from the group consisting of poly(ethylene glycol), hydroxyethyl starch, XTEN, and a proline-alanine-serine polymer.

Other non-limiting examples of polymers are provided in WO 2019/236567, incorporated herein by reference.

### Enzyme-cleavable Linkers

A therapeutic conjugate, in some embodiments, comprises an enzyme-cleavable linker, which links the half-life extension moiety to a drug moiety. The linker (e.g., the substrate recognition sequence (SRS) of the linker) is selectively cleaved in the vicinity of the target cells so that the free drug moiety is released from the conjugate in the vicinity of the target cells so as to exert its pharmacological activities preferentially on the cells/tissue nearby to the target cells, rather than on wanted (healthy) cells. Thus, in some embodiments, the SRS is selectively cleaved such that the drug moiety is released as the free drug moiety in the vicinity of the target cells at least five times or ten times more than the extent to which the free drug moiety it is released in the vicinity of healthy cells/tissues, and in some embodiments, at least 100 or 500 or 1000 times more.

For a given target cell, the skilled person will be able to identify appropriate SRS that is selectively cleavable in the vicinity of the target cell, using established methods in the art. For example, which proteases cleave which peptides can be assessed by consulting peptide libraries and studying an MS analysis of the fragmentation profile following cleavage. Also, published literature of protease cleavage motifs and peptide cleavage data can be searched as described further below.

In some aspects, the SRS is a protease cleavage site. Thus, when the target cells are tumor cells, the SRS may be cleavable selectively by proteases that reside in the vicinity of the tumor cells. Thus, the SRS may be one that is cleavable by a tumor associated protease. It is well known that during tumor development, tumors aberrantly express proteases which allow them to invade local tissues and eventually metastasize.

For example, the protease may be present extracellularly in the diseased state tissue in a subject at levels at least 2, 3, 4, 5, 6, 7, 8, 9, 10, 15, 20, 30, 40, 50, 60, 70, 80, 90, or 100 times greater than the healthy state of the tissue in the subject.

As another example, the protease may be present extracellularly in the diseased state of the tissue in a subject at levels at least 2, 3, 4, 5, 6, 7, 8, 9, 10, 15, 20, 30, 40, 50, 60, 70, 80, 90 or 100 times greater than other tissue of the subject.

In some embodiments, the protease is a serine protease, metal protease, or cysteine protease.

The protease may be a metalloproteinase (MMP1-28) including both membrane bound (MMP14-17 and MMP24-25) and secreted forms (MMP1-13 and MMP 18-23 and MMP26-28). The protease may belong to the A Disintegrin and Metalloproteinase (ADAM) and A Disintegrin, or Metalloproteinase with Thrombospondin Motifs (ADAMTS) families of proteases. Other examples include CD 10 (CALLA) and prostate specific antigen (PSA). It is appreciated that the proteases may or may not be membrane bound.

Protease cleavage sites are well known in the scientific literature, and can readily serve as the basis for a given SRS being included in the drug-conjugate moieties using established synthetic techniques known in the art.

To the extent representing a protease whose extracellular concentration is upregulated/increased in the target tissue by changes in expression, cellular trafficking or, in the case of intracellular enzymes that may become extracellular, by cell lysis caused by the disease state, SRS may utilized which are designed to be selectively cleavable by one or a select sub-group of human proteases selected from the group consisting of (MEROPS peptidase database number provided in parentheses; Rawlings N. D., Morton F. R., Kok, C. Y., Kong, J. & Barrett A. J. (2008) MEROPS: the peptidase database. Nucleic Acids Res. 36 Database issue, D320-325): pepsin A (MER000885), gastricsin (MER000894), memapsin-2 (MER005870), renin (MER000917), cathepsin D (MER000911), cathepsin E (MER000944), memapsin-1 (MER005534), napsin A (MER004981), Mername-AA034 peptidase (MER014038), pepsin A4 (MER037290), pepsin A5 (Homo sapiens) (MER037291), hCG1733572 (Homo sapiens)-type putative peptidase (MER107386), napsin B pseudogene (MER004982), CYMP g.p. (Homo sapiens) (MER002929), subfamily A1A unassigned peptidases (MER181559), mouse mammary tumor virus retropepsin (MER048030), rabbit endogenous retrovirus endopeptidase (MER043650), S71-related human endogenous retropepsin (MER001812), RTVL-H-type putative peptidase (MER047117), RTVL-H-type putative peptidase (MER047133), RTVL-H-type putative peptidase (MER047160), RTVL-H-type putative peptidase (MER047206), RTVL-H-type putative peptidase (MER047253), RTVL-H-type putative peptidase (MER047260), RTVL-H-type putative peptidase (MER047291), RTVL-H-type putative peptidase (MER047418), RTVL-H-type putative peptidase (MER047440), RTVL-H-type putative peptidase (MER047479), RTVL-H-type putative peptidase (MER047559), RTVL-H-type putative peptidase (MER047583), RTVL-H-type putative peptidase (MERO 15446), human endogenous retrovirus retropepsin homologue 1 (MERO 15479), human endogenous retrovirus retropepsin homologue 2 (MERO 15481), endogenous retrovirus retropepsin pseudogene 1 (Homo sapiens chromosome 14) (MER029977), endogenous retrovirus retropepsin pseudogene 2 (Homo sapiens chromosome 8) (MER029665), endogenous retrovirus retropepsin pseudogene 3 (Homo sapiens chromosome 17) (MER002660), endogenous retrovirus retropepsin pseudogene 3 (Homo sapiens chromosome 17) (MER030286), endogenous retrovirus retropepsin pseudogene 3 (Homo sapiens chromosome 17) (MER047144), endogenous retrovirus retropepsin pseudogene 5 (Homo sapiens chromosome 12) (MER029664), endogenous retrovirus retropepsin pseudogene 6 (Homo sapiens chromosome 7) (MER002094), endogenous retrovirus retropepsin pseudogene 7 (Homo sapiens chromosome 6) (MER029776), endogenous retrovirus retropepsin pseudogene 8 (Homo sapiens chromosome Y) (MER030291), endogenous retrovirus retropepsin pseudogene 9 (Homo sapiens chromosome 19) (MER029680), endogenous retrovirus retropepsin pseudogene 10 (Homo sapiens chromosome 12) (MER002848), endogenous retrovirus retropepsin pseudogene 11 (Homo sapiens chromosome 17) (MER004378), endogenous retrovirus retropepsin pseudogene 12 (Homo sapiens chromosome 11) (MER003344), endogenous retrovirus retropepsin pseudogene 13 (Homo sapiens chromosome 2 and similar) (MER029779), endogenous retrovirus retropepsin pseudogene 14 (Homo sapiens chromosome 2) (MER029778), endogenous retrovirus retropepsin pseudogene 15 (Homo sapiens chromosome 4) (MER047158), endogenous retrovirus retropepsin pseudogene 15 (Homo sapiens chromosome 4) (MER047332), endogenous retrovirus retropepsin pseudogene 15 (Homo sapiens chromosome 4) (MER003182), endogenous retrovirus retropepsin pseudogene 16 (MER047165), endogenous retrovirus retropepsin pseudogene 16 (MER047178), endogenous retrovirus retropepsin pseudogene 16 (MER047200), endogenous retrovirus retropepsin pseudogene 16 (MER047315), endogenous retrovirus retropepsin pseudogene 16 (MER047405), endogenous retrovirus retropepsin pseudogene 16 (MER030292), endogenous retrovirus retropepsin pseudogene 17 (Homo sapiens chromosome 8) (MER005305), endogenous retrovirus retropepsin pseudogene 18 (Homo sapiens chromosome 4) (MER030288), endogenous retrovirus retropepsin pseudogene 19 (Homo sapiens chromosome 16) (MER001740), endogenous retrovirus retropepsin pseudogene 21 (Homo sapiens) (MER047222), endogenous retrovirus retropepsin pseudogene 21 (Homo sapiens) (MER047454), endogenous retrovirus retropepsin pseudogene 21 (Homo sapiens) (MER047477), endogenous retrovirus retropepsin pseudogene 21

(Homo sapiens) (MER004403), endogenous retrovirus retropepsin pseudogene 22 (Homo sapiens chromosome X) (MER030287), subfamily A2A non peptidase homologues (MER047046), subfamily A2A non-peptidase homologues (MER047052), subfamily A2A non-peptidase homologues (MER047076), subfamily A2A non-peptidase homologues (MER047080), subfamily A2A non-peptidase homologues (MER047088), subfamily A2A non-peptidase homologues (MER047089), subfamily A2A non-peptidase homologues (MER047091), subfamily A2A non-peptidase homologues (MER047092), subfamily A2A non-peptidase homologues (MER047093), subfamily A2A non-peptidase homologues (MER047094), subfamily A2A non-peptidase homologues (MER047097), subfamily A2A non-peptidase homologues (MER047099), subfamily A2A non-peptidase homologues (MER047101), subfamily A2A non-peptidase homologues (MER047102), subfamily A2A non-peptidase homologues (MER047107), subfamily A2A non-peptidase homologues (MER047108), subfamily A2A non-peptidase homologues (MER047109), subfamily A2A non-peptidase homologues (MER047110), subfamily A2A non-peptidase homologues (MER047111), subfamily A2A non-peptidase homologues (MER047114), subfamily A2A non-peptidase homologues (MER047118), subfamily A2A non-peptidase homologues (MER047121), subfamily A2A non-peptidase homologues (MER047122), subfamily A2A non-peptidase homologues (MER047126), subfamily A2A non-peptidase homologues (MER047129), subfamily A2A non-peptidase homologues (MER047130), subfamily A2A non-peptidase homologues (MER047134), subfamily A2A non-peptidase homologues (MER047135), subfamily A2A non-peptidase homologues (MER047137), subfamily A2A non-peptidase homologues (MER047140), subfamily A2A non-peptidase homologues (MER047141), subfamily A2A non-peptidase homologues (MER047142), subfamily A2A non-peptidase homologues (MER047148), subfamily A2A non-peptidase homologues (MER047149), subfamily A2A non-peptidase homologues (MER047151), subfamily A2A non-peptidase homologues (MER047154), subfamily A2A non-peptidase homologues (MER047155), subfamily A2A non-peptidase homologues (MER047156), subfamily A2A non-peptidase homologues (MER047157), subfamily A2A non-peptidase homologues (MER047159), subfamily A2A non-peptidase homologues (MER047161), subfamily A2A non-peptidase homologues (MER047163), subfamily A2A non-peptidase homologues (MER047166), subfamily A2A non-peptidase homologues (MER047171), subfamily A2A non-peptidase homologues (MER047173), subfamily A2A non-peptidase homologues (MER047174), subfamily A2A non-peptidase homologues (MER047179), subfamily A2A non-peptidase homologues (MER047183), subfamily A2A non-peptidase homologues (MER047186), subfamily A2A non-peptidase homologues (MER047190), subfamily A2A non-peptidase homologues (MER047191), subfamily A2A non-peptidase homologues (MER047196), subfamily A2A non-peptidase homologues (MER047198), subfamily A2A non-peptidase homologues (MER047199), subfamily A2A non-peptidase homologues (MER047201), subfamily A2A non-peptidase homologues (MER047202), subfamily A2A non-peptidase homologues (MER047203), subfamily A2A non-peptidase homologues (MER047204), subfamily A2A non-peptidase homologues (MER047205), subfamily A2A non-peptidase homologues (MER047207), subfamily A2A non-peptidase homologues (MER047208), subfamily A2A non-peptidase homologues (MER047210), subfamily A2A non-peptidase homologues (MER047211), subfamily A2A non-peptidase homologues (MER047212), subfamily A2A non-peptidase homologues (MER047213), subfamily A2A non-peptidase homologues (MER047215), subfamily A2A non-peptidase homologues (MER047216), subfamily A2A non-peptidase homologues (MER047218), subfamily A2A non-peptidase homologues (MER047219), subfamily A2A non-peptidase homologues (MER047221), subfamily A2A non-peptidase homologues (MER047224), subfamily A2A non-peptidase homologues (MER047225), subfamily A2A non-peptidase homologues (MER047226), subfamily A2A non-peptidase homologues (MER047227), subfamily A2A non-peptidase homologues (MER047230), subfamily A2A non-peptidase homologues (MER047232), subfamily A2A non-peptidase homologues (MER047233), subfamily A2A non-peptidase homologues (MER047234), subfamily A2A non-peptidase homologues (MER047236), subfamily A2A non-peptidase homologues (MER047238), subfamily A2A non-peptidase homologues (MER047239), subfamily A2A non-peptidase homologues (MER047240), subfamily A2A non-peptidase homologues (MER047242), subfamily A2A non-peptidase homologues (MER047243), subfamily A2A non-peptidase homologues (MER047249), subfamily A2A non-peptidase homologues (MER047251), subfamily A2A non-peptidase homologues (MER047252), subfamily A2A non-peptidase homologues (MER047254), subfamily A2A non-peptidase homologues (MER047255), subfamily A2A non-peptidase homologues (MER047263), subfamily A2A non-peptidase homologues (MER047265), subfamily A2A non-peptidase homologues (MER047266), subfamily A2A non-peptidase homologues (MER047267), subfamily A2A non-peptidase homologues (MER047268), subfamily A2A non-peptidase homologues (MER047269), subfamily A2A non-peptidase homologues (MER047272), subfamily A2A non-peptidase homologues (MER047273), subfamily A2A non-peptidase homologues (MER047274), subfamily A2A non-peptidase homologues (MER047275), subfamily A2A non-peptidase homologues (MER047276), subfamily A2A non-peptidase homologues (MER047279), subfamily A2A non-peptidase homologues (MER047280), subfamily A2A non-peptidase homologues (MER047281), subfamily A2A non-peptidase homologues (MER047282), subfamily A2A non-peptidase homologues (MER047284), subfamily A2A non-peptidase homologues (MER047285), subfamily A2A non-peptidase homologues (MER047289), subfamily A2A non-peptidase homologues (MER047290), subfamily A2A non-peptidase homologues (MER047294), subfamily A2A non-peptidase homologues (MER047295), subfamily A2A non-peptidase homologues (MER047298), subfamily A2A non-peptidase homologues (MER047300), subfamily A2A non-peptidase homologues (MER047302), subfamily A2A non-peptidase homologues (MER047304), subfamily A2A non-peptidase homologues (MER047305), subfamily A2A non-peptidase homologues (MER047306), subfamily A2A non-peptidase homologues (MER047307), subfamily A2A non-peptidase homologues (MER047310), subfamily A2A non-peptidase homologues (MER047311), subfamily A2A non-peptidase homologues (MER047314), subfamily A2A non-peptidase homologues (MER047318), subfamily A2A non-peptidase homologues (MER047320), subfamily A2A non-peptidase homologues (MER047321), subfamily A2A non-peptidase homologues (MER047322), subfamily A2A non-peptidase homologues (MER047326), subfamily A2A non-peptidase homologues (MER047327), subfamily A2A non-peptidase homologues (MER047330), subfamily A2A non-peptidase homologues (MER047333), subfamily A2A non-peptidase homologues (MER047362), subfamily A2A non-peptidase homologues (MER047366), subfamily A2A non-peptidase homologues (MER047369), subfamily A2A non-peptidase homologues (MER047370), subfamily A2A non-peptidase homologues (MER047371), subfamily A2A non-peptidase homologues (MER047375), subfamily A2A non-peptidase homologues (MER047376), subfamily A2A non-peptidase homologues (MER047381), subfamily A2A non-peptidase homologues (MER047383), subfamily A2A non-peptidase homologues (MER047384), subfamily A2A non-peptidase homologues (MER047385), subfamily A2A non-peptidase homologues (MER047388), subfamily A2A non-peptidase homologues (MER047389), subfamily A2A non-peptidase homologues (MER047391), subfamily A2A non-peptidase homologues (MER047394), subfamily A2A non-peptidase homologues (MER047396), subfamily A2A non-peptidase homologues (MER047400), subfamily A2A non-peptidase homologues (MER047401), subfamily A2A non-peptidase homologues (MER047403), subfamily A2A non-peptidase homologues (MER047406), subfamily A2A non-peptidase homologues (MER047407), subfamily A2A non-peptidase homologues (MER047410), subfamily A2A non-peptidase homologues (MER047411), subfamily A2A non-peptidase homologues (MER047413), subfamily A2A non-peptidase homologues (MER047414), subfamily A2A non-peptidase homologues (MER047416), subfamily A2A non-peptidase homologues (MER047417), subfamily A2A non-peptidase homologues (MER047420), subfamily A2A non-peptidase homologues (MER047423), subfamily A2A non-peptidase homologues (MER047424), subfamily A2A non-peptidase homologues (MER047428), subfamily A2A non-peptidase homologues (MER047429), subfamily A2A non-peptidase homologues (MER047431), subfamily A2A non-peptidase homologues (MER047434), subfamily A2A non-peptidase homologues (MER047439), subfamily A2A non-peptidase homologues (MER047442), subfamily A2A non-peptidase homologues (MER047445), subfamily A2A non-peptidase homologues (MER047449), subfamily A2A non-peptidase homologues (MER047450), subfamily A2A non-peptidase homologues (MER047452), subfamily A2A non-peptidase homologues (MER047455), subfamily A2A non-peptidase homologues (MER047457), subfamily A2A non-peptidase homologues (MER047458), subfamily A2A non-peptidase homologues (MER047459), subfamily A2A non-peptidase homologues (MER047463), subfamily A2A non-peptidase homologues (MER047468), subfamily A2A non-peptidase homologues (MER047469), subfamily A2A non-peptidase homologues (MER047470), subfamily A2A non-peptidase homologues (MER047476), subfamily A2A non-peptidase homologues (MER047478), subfamily A2A non-peptidase homologues (MER047483), subfamily A2A non-peptidase homologues (MER047488), subfamily A2A non-peptidase homologues (MER047489), subfamily A2A non-peptidase homologues (MER047490), subfamily A2A non-peptidase homologues (MER047493), subfamily A2A non-peptidase homologues (MER047494), subfamily A2A non-peptidase homologues (MER047495), subfamily A2A non-peptidase homologues (MER047496), subfamily A2A non-peptidase homologues (MER047497), subfamily A2A non-peptidase homologues (MER047499), subfamily A2A non-peptidase homologues (MER047502), subfamily A2A non-peptidase homologues (MER047504), subfamily A2A non-peptidase homologues (MER047511), subfamily A2A non-peptidase homologues (MER047513), subfamily A2A non-peptidase homologues (MER047514), subfamily A2A non-peptidase homologues (MER047515), subfamily A2A non-peptidase homologues (MER047516), subfamily A2A non-peptidase homologues (MER047520), subfamily A2A non-peptidase homologues (MER047533), subfamily A2A non-peptidase homologues (MER047537), subfamily A2A non-peptidase homologues (MER047569), subfamily A2A non-peptidase homologues (MER047570), subfamily A2A non-peptidase homologues (MER047584), subfamily A2A non-peptidase homologues (MER047603), subfamily A2A non-peptidase homologues (MER047604), subfamily A2A non-peptidase homologues (MER047606), subfamily A2A non-peptidase homologues (MER047609), subfamily A2A non-peptidase homologues (MER047616), subfamily A2A non-peptidase homologues (MER047619), subfamily A2A non-peptidase homologues (MER047648), subfamily A2A non-peptidase homologues (MER047649), subfamily A2A non-peptidase homologues (MER047662), subfamily A2A non-peptidase homologues (MER048004), subfamily A2A non-peptidase homologues (MER048018), subfamily A2A non-peptidase homologues (MER048019), subfamily A2A non-peptidase homologues (MER048023), subfamily A2A non-peptidase homologues (MER048037), subfamily A2A unassigned peptidases (MER047164), subfamily A2A unassigned peptidases (MER047231), subfamily A2A unassigned peptidases (MER047386), skin aspartic protease (MER057097), presenilin 1 (MER005221), presenilin 2

(MER005223), impas 1 peptidase (MER019701), impas 1 peptidase (MER184722), impas 4 peptidase (MER019715), impas 2 peptidase (MERO 19708), impas 5 peptidase (MER019712), impas 3 peptidase (MER019711), possible family A22 pseudogene (Homo sapiens chromosome 18) (MER029974), possible family A22 pseudogene (Homo sapiens chromosome 11) (MER023159), cathepsin V (MER004437), cathepsin X (MER004508), cathepsin F (MER004980), cathepsin L (MER000622), cathepsin S (MER000633), cathepsin 0 (MER001690), cathepsin K (MER000644), cathepsin W (MER003756), cathepsin H (MER000629), cathepsin B (MER000686), dipeptidyl-peptidase I (MER001937), bleomycin hydrolase (animal) (MER002481), tubulointerstitial nephritis antigen (MER016137), tubulointerstitial nephritis antigen-related protein (MER021799), cathepsin L-like pseudogene 1 (Homo sapiens) (MER002789), cathepsin B-like pseudogene (chromosome 4, Homo sapiens) (MER029469), cathepsin B-like pseudogene (chromosome 1, Homo sapiens) (MER029457), CTSLL2 g.p. (Homo sapiens) (MER005210), CTSLL3 g.p. (Homo sapiens) (MER005209), calpain-1 (MER000770), calpain-2 (MER000964), calpain-3 (MER001446), calpain-9 (MER004042), calpain-8 (MER021474), calpain-1 5 (MER004745), calpain-5 (MER002939), calpain-11 (MER005844), calpain-12 (MER029889), calpain-10 (MER013510), calpain-13 (MER020139), calpain-14 (MER029744), Mername-AA253 peptidase (MER005537), calpamodulin (MER000718), hypothetical protein flj40251 (MER003201), ubiquitinyl hydrolase-L1 (MER000832), ubiquitinyl hydrolase-L3 (MER000836), ubiquitinyl hydrolase-BAP1 (MER003989), ubiquitinyl hydrolase-UCH37 (MER005539), ubiquitin-specific peptidase 5 (MER002066), ubiquitin-specific peptidase 6 (MER000863), ubiquitin-specific peptidase 4 (MEROO 1795), ubiquitin-specific peptidase 8 (MER001884), ubiquitin-specific peptidase 13 (MER002627), ubiquitin-specific peptidase 2 (MER004834), ubiquitin-specific peptidase 11 (MER002693), ubiquitin-specific peptidase 14 (MER002667), ubiquitin-specific peptidase 7 (MER002896), ubiquitin-specific peptidase 9X (MER005877), ubiquitin-specific peptidase 10 (MER004439), ubiquitin-specific peptidase 1 (MER004978), ubiquitin-specific peptidase 12 (MER005454), ubiquitin-specific peptidase 16 (MER005493), ubiquitin-specific peptidase 15 (MER005427), ubiquitin-specific peptidase 17 (MER002900), ubiquitin-specific peptidase 19 (MER005428), ubiquitin-specific peptidase 20 (MER005494), ubiquitin-specific peptidase 3 (MER005513), ubiquitin-specific peptidase 9Y (MER004314), ubiquitin-specific peptidase 18 (MER005641), ubiquitin-specific peptidase 21 (MER006258), ubiquitin-specific peptidase 22 (MER012130), ubiquitin-specific peptidase 33 (MER014335), ubiquitin-specific peptidase 29 (MER012093), ubiquitin-specific peptidase 25 (MER011115), ubiquitin-specific peptidase 36 (MER014033), ubiquitin-specific peptidase 32 (MER014290), ubiquitin-specific peptidase 26 (Homo sapiens-type) (MERO 14292), ubiquitin-specific peptidase 24 (MER005706), ubiquitin-specific peptidase 42 (MER011852), ubiquitin-specific peptidase 46 (MER014629), ubiquitin-specific peptidase 37 (MER014633), ubiquitin-specific peptidase 28 (MER014634), ubiquitin-specific peptidase 47 (MERO 14636), ubiquitin-specific peptidase 38 (MERO 14637), ubiquitin-specific peptidase 44 (MER014638), ubiquitin-specific peptidase 50 (MER030315), ubiquitin-specific peptidase 35 (MERO 14646), ubiquitin-specific peptidase 30 (MERO 14649), Mername-AA091 peptidase (MER014743), ubiquitin-specific peptidase 45 (MER030314), ubiquitin-specific peptidase 51 (MER014769), ubiquitin-specific peptidase 34 (MER014780), ubiquitin-specific peptidase 48 (MER064620), ubiquitin-specific peptidase 40 (MERO 15483), ubiquitin-specific peptidase 41 (MER045268), ubiquitin-specific peptidase 31 (MER015493), Mername-AA129 peptidase (MER016485), ubiquitin-specific peptidase 49 (MER016486), Mername-AA187 peptidase (MER052579), ETSP17-like peptidase (MER030192), ubiquitin-specific peptidase 54 (MER028714), ubiquitin-specific peptidase 53 (MER027329), ubiquitin-specific endopeptidase 39 [misleading] (MER064621), Memame-AA090 non-peptidase homologue (MERO 14739), ubiquitin-specific peptidase [misleading] (MER030140), ubiquitin-specific peptidase 52 [misleading] (MER030317), NEK2 pseudogene (MER014736), C19 pseudogene (Homo sapiens: chromosome 5) (MER029972), Memame-AA088 peptidase (MER014750), autophagin-2 (MER013564), autophagin-1 (MER013561), autophagin-3 (MER014316), autophagin-4 (MER064622), Cezanne deubiquitinylating peptidase (MER029042), Cezanne-2 peptidase (MER029044), tumor necrosis factor alpha-induced protein 3 (MER029050), trabid peptidase (MER029052), VCIP135 deubiquitinating peptidase (MER152304), otubain-1 (MER029056), otubain-2 (MER029061), CylD protein (MER030104), UfSP1 peptidase (MER042724), ETfSP2 peptidase (MER060306), DEIBA deubiquitinylating enzyme (MER086098), KIAA0459 (Homo sapiens)-like protein (MER122467), Otud1 protein (MER125457), glycosyltransferase 28 domain containing 1, isoform CRA c (Homo sapiens)-like (MER123606), hin1L g.p. (Homo sapiens) (MER139816), ataxin-3 (MER099998), ATXN3L putative peptidase (MER115261), Josephin domain containing 1 (Homo sapiens) (MER125334), Josephin domain containing 2 (Homo sapiens) (MER124068), YOD1 peptidase (MER116559), legumain (plant alpha form) (MER044591), legumain (MER001800), glycosylphosphatidylinositokprotein transamidase (MER002479), legumain pseudogene (Homo sapiens) (MER029741), family C13 unassigned peptidases (MER175813), caspase-1 (MER000850), caspase-3 (MER000853), caspase-7 (MER002705), caspase-6 (MER002708), caspase-2 (MER001644), caspase-4 (MER001938), caspase-5 (MER002240), caspase-8 (MER002849), caspase-9 (MER002707), caspase-lO (MER002579), caspase-14 (MER012083), paracaspase (MER019325), Memame-AA143 peptidase (MER021304), Mername-AA186 peptidase (MER020516), putative caspase (Homo sapiens) (MER021463), FLIP protein (MER003026), Memame-AA142 protein (MER021316), caspase-12 pseudogene (Homo sapiens) (MER019698), Mername-AA093 caspase pseudogene (MER014766), subfamily C14A non-peptidase homologues (MER185329), subfamily C14A non-peptidase homologues (MER179956), separase (Homo sapiens-type) (MER011775), separase-like pseudogene (MER014797), SENP1 peptidase (MER011012), SENP3 peptidase (MER011019), SENP6 peptidase (MER011109), SENP2 peptidase (MER012183), SENP5 peptidase (MER014032), SENP7 peptidase (MER014095), SENP8 peptidase (MER016161), SENP4 peptidase (MER005557), pyroglutamyl-peptidase I (chordate) (MER011032), Memame-AA073 peptidase (MER029978), Sonic hedgehog protein (MER002539), Indian hedgehog protein (MER002538), Desert hedgehog protein (MER012170), dipeptidyl-peptidase III (MER004252), Mername-AA164 protein (MER020410), LOC138971 g.p. (Homo sapiens) (MER020074), Atp23 peptidase (MER060642), prenyl peptidase 1 (MER004246), aminopeptidase N (MER000997), aminopeptidase A (MER001012), leukotriene A4 hydrolase (MER001013), pyroglutamyl-peptidase II (MER012221), cytosol alanyl aminopeptidase (MER002746), cystinyl aminopeptidase (MER002060), aminopeptidase B (MER001494), aminopeptidase PILS (MER005331), arginyl aminopeptidase-like 1 (MERO 12271), leukocyte-derived arginine aminopeptidase (MER002968), aminopeptidase Q (MER052595), aminopeptidase 0 (MER019730), Tata binding protein associated factor (MER026493), angiotensin-converting enzyme peptidase unit 1 (MER004967), angiotensin-converting enzyme peptidase unit 2 (MER001019), angiotensin-converting enzyme-2 (MER011061), Memame-AA153 protein (MER020514), thimet oligopeptidase (MER001737), neurolysin (MERO 10991), mitochondrial intermediate peptidase (MER003665), Mername-AA154 protein (MER021317), leishmanolysin-2 (MER014492), leishmanolysin-3 (MER180031), matrix metallopeptidase-1 (MER001063), matrix metallopeptidase-8 (MER001084), matrix metallopeptidase-2 (MER001080), matrix metallopeptidase-9 (MER001085), matrix metallopeptidase-3 (MER001068), matrix metallopeptidase-lO (Homo sapiens-type) (MER001072), matrix metallopeptidase-1 1 (MER001075), matrix metallopeptidase-7 (MER001092), matrix metallopeptidase-1 2 (MER001089), matrix metallopeptidase-1 3 (MER001411), membrane-type matrix metallopeptidase-1 (MER001077), membrane-type matrix metallopeptidase-2 (MER002383), membrane-type matrix metallopeptidase-3 (MER002384), membrane-type matrix metallopeptidase-4 (MER002595), matrix metallopeptidase-20 (MER003021), matrix metallopeptidase-1 9 (MER002076), matrix metallopeptidase-23B (MER004766), membrane-type matrix metallopeptidase-5 (MER005638), membrane-type matrix metallopeptidase-6 (MERO 12071), matrix metallopeptidase-21 (MER006101), matrix metallopeptidase-22 (MERO 14098), matrix metallopeptidase-26 (MERO 12072), matrix metallopeptidase-28 (MER013587), matrix metallopeptidase-23A (MER037217), macrophage elastase homologue (chromosome 8, Homo sapiens) (MER030035), Memame-AA156 protein (MER021309), matrix metallopeptidase-like 1 (MER045280), subfamily M10A non-peptidase homologues (MER175912), subfamily M10A non-peptidase homologues (MER187997), subfamily M10A non-peptidase homologues (MER187998), subfamily M10A non-peptidase homologues (MER180000), meprin alpha subunit (MER001111), meprin beta subunit (MER005213), procollagen C-peptidase (MER001113), mammalian tolloid-like 1 protein (MER005124), mammalian-type tolloid-like 2 protein (MER005866), ADAMTS9 peptidase (MER012092), ADAMTS14 peptidase (MER016700), ADAMTS15 peptidase (MERO 17029), ADAMTS16 peptidase (MER015689), ADAMTS17 peptidase (MERO 16302), ADAMTS18 peptidase (MERO 16090), ADAMTS19 peptidase (MERO 15663), ADAMS peptidase (MER003902), ADAM9 peptidase (MER001140), ADAM 10 peptidase (MER002382), ADAM 12 peptidase (MER005107), ADAM 19 peptidase (MERO 12241), ADAM 15 peptidase (MER002386), ADAM 17 peptidase (MER003094), ADAM20 peptidase (MER004725), ADAMDEC1 peptidase (MER000743), ADAMTS3 peptidase (MER005100), ADAMTS4 peptidase (MER005101), ADAMTS1 peptidase (MER005546), ADAM28 peptidase (Homo sapiens-type) (MER005495), ADAMTS5 peptidase (MER005548), ADAMTS8 peptidase (MER005545), ADAMTS6 peptidase (MER005893), ADAMTS7 peptidase (MER005894), ADAM30 peptidase (MER006268), ADAM21 peptidase (Homo sapiens-type) (MER004726), ADAMTS10 peptidase (MER014331), ADAMTS 12 peptidase (MER014337), ADAMTS13 peptidase (MER015450), ADAM33 peptidase (MER015143), ovastacin (MER029996), ADAMTS20 peptidase (Homo sapiens-type) (MER026906), procollagen I N-peptidase (MER004985), ADAM2 protein (MER003090), ADAM6 protein (MER047044), ADAM7 protein (MER005109), ADAM18 protein (MER012230), ADAM32 protein (MER026938), non-peptidase homologue (Homo sapiens chromosome 4) (MER029973), family M12 non-peptidase homologue (Homo sapiens chromosome 16) (MER047654), family M12 non-peptidase homologue (Homo sapiens chromosome 15) (MER047250), ADAM3B protein (Homo sapiens-type) (MER005199), ADAM11 protein (MER001146), ADAM22 protein (MER005102), ADAM23 protein (MER005103), ADAM29 protein (MER006267), protein similar to ADAM21 peptidase preproprotein (Homo sapiens) (MER026944), Memame-AA225 peptidase homologue (Homo sapiens) (MER047474), putative ADAM pseudogene (chromosome 4, Homo sapiens) (MER029975), ADAM3A g.p. (Homo sapiens) (MER005200), ADAM1 g.p. (Homo sapiens) (MER003912), subfamily M12B non peptidase homologues (MER188210), subfamily M12B non-peptidase homologues (MER188211), subfamily M12B non-peptidase homologues (MER188212), subfamily M12B non-peptidase homologues (MER188220), neprilysin (MER001050), endothelin-converting enzyme 1 (MEROO 1057), endothelin-converting enzyme 2 (MER004776), DINE peptidase (MER005197), neprilysin-2 (MER013406), Kell blood-group protein (MEROO 1054), PHEX peptidase (MER002062), i-AAA peptidase (MEROO 1246), i-AAA peptidase (MER005755), paraplegin (MER004454), Afg3-like protein 2 (MER005496), Afg3-like protein 1A (MER014306), pappalysin-1 (MER002217), pappalysin-2 (MER014521), famesylated-protein converting enzyme 1 (MER002646), metalloprotease-related protein-1 (MER030873), aminopeptidase AMZ2 (MER011907), aminopeptidase AMZ1 (MER058242), carboxypeptidase A1 (MER001190), carboxypeptidase A2 (MEROO 1608), carboxypeptidase B (MEROO 1194), carboxypeptidase N (MEROO 1198), carboxypeptidase E (MEROO 1199), carboxypeptidase M (MEROO 1205), carboxypeptidase U (MEROO 1193), carboxypeptidase A3 (MEROO 1187), metallocarboxypeptidase D peptidase unit 1 (MER003781), metallocarboxypeptidase Z (MER003428), metallocarboxypeptidase D peptidase unit 2 (MER004963), carboxypeptidase A4 (MER013421), carboxypeptidase A6 (MER013456), carboxypeptidase A5 (MER017121), metallocarboxypeptidase 0 (MER016044), cytosolic carboxypeptidase-like protein 5 (MER033174), cytosolic carboxypeptidase 3 (MER033176), cytosolic carboxypeptidase 6 (MER033178), cytosolic carboxypeptidase 1 (MER033179), cytosolic carboxypeptidase 2 (MER037713), metallocarboxypeptidase D non-peptidase unit (MER004964), adipocyte-enhancer binding protein 1 (MER003889), carboxypeptidase-like protein XI (MER013404), carboxypeptidase-like protein X2 (MER078764), cytosolic carboxypeptidase (MER026952), family M14 non-peptidase homologues (MER199530), insulysin (MER001214), mitochondrial processing peptidase beta-subunit (MER004497), nardilysin (MER003883), eupitrilysin (MER004877), mitochondrial processing peptidase non-peptidase alpha subunit (MER001413), ubiquinol-cytochrome c reductase core protein I (MER003543), ubiquinol-cytochrome c reductase core protein II (MER003544), ubiquinol-cytochrome c reductase core protein domain 2 (MER043998), insulysin unit 2 (MER046821), nardilysin unit 2 (MER046874), insulysin unit 3 (MER078753), mitochondrial processing peptidase subunit alpha unit 2 (MER124489), nardilysin unit 3 (MER142856), LOC133083 g.p. (Homo sapiens) (MER021876), subfamily M16B non-peptidase homologues (MER188757), leucyl aminopeptidase (animal) (MER003100), Mername-AA040 peptidase (MER003919), leucyl aminopeptidase-1 (Caenorhabditis-type) (MER013416), methionyl aminopeptidase 1 (MEROO 1342), methionyl aminopeptidase 2 (MER001728), aminopeptidase P2 (MER004498), Xaa-Pro dipeptidase (eukaryote) (MEROO 1248), aminopeptidase P1 (MER004321), mitochondrial intermediate cleaving peptidase 55 kDa (MER013463), mitochondrial methionyl aminopeptidase (MER014055), Mername-AA020 peptidase homologue (MERO 10972), proliferation-association protein 1 (MER005497), chromatin-specific transcription elongation factor 140 kDa subunit (MER026495), proliferation-associated protein 1-like (Homo sapiens chromosome X) (MER029983), Mername-AA226 peptidase homologue (Homo sapiens) (MER056262), Mername-AA227 peptidase homologue (Homo sapiens) (MER047299), subfamily M24A non-peptidase homologues (MER179893), aspartyl aminopeptidase (MER003373), Gly-Xaa carboxypeptidase (MER033182), carnosine dipeptidase II (MERO 14551), carnosine dipeptidase I (MER015142), Memame-AA161 protein (MER021873), aminoacylase (MER001271), glutamate carboxypeptidase II (MER002104), NAALADASE L peptidase (MER005239), glutamate carboxypeptidase III (MER005238), plasma glutamate carboxypeptidase (MER005244), Mername-AA103 peptidase (MER015091), Fxna peptidase (MER029965), transferrin receptor protein (MER002105), transferrin receptor 2 protein (MER005152), glutaminyl cyclise (MERO 15095), glutamate carboxypeptidase II (Homo sapiens)-type non peptidase homologue (MER026971), nicalin (MER044627), membrane dipeptidase (MER001260), membrane-bound dipeptidase-2 (MER013499), membrane-bound dipeptidase-3 (MERO 13496), dihydro-orotase (MER005767), dihydropyrimidinase (MER033266), dihydropyrimidinase related protein-1 (MER030143), dihydropyrimidinase related protein-2 (MER030155), dihydropyrimidinase related protein-3 (MER030151), dihydropyrimidinase related protein-4 (MER030149), dihydropyrimidinase related protein-5 (MER030136), hypothetical protein like 5730457F11RIK (MER033184), 1300019j08rik protein (MER033186)), guanine aminohydrolase (MER037714), Kael putative peptidase (MEROO 1577), OSGEPL1-like protein (MER013498), S2P peptidase (MER004458), subfamily M23B non-peptidase homologues (MER199845), subfamily M23B non-peptidase homologues (MER199846), subfamily M23B non-peptidase homologues (MER199847), subfamily M23B non-peptidase homologues (MER137320), subfamily M23B non-peptidase homologues (MER201557), subfamily M23B non-peptidase homologues (MER199417), subfamily M23B non-peptidase homologues (MER199418), subfamily M23B non-peptidase homologues (MER199419), subfamily M23B non-peptidase homologues (MER199420), subfamily M23B non-peptidase homologues (MER175932), subfamily M23B non-peptidase homologues (MER199665), Pohl peptidase (MER020382), Jab1/MPN domain metalloenzyme (MER022057), Mername-AA165 peptidase (MER021865), Brcc36 isopeptidase (MER021890), histone H2A deubiquitinase MYSM1 (MER021887), AMSH deubiquitinating peptidase (MER030146), putative peptidase (Homo sapiens chromosome 2) (MER029970), Memame-AA168 protein (MER021886), COP9 signalosome subunit 6 (MER030137), 26S proteasome non-ATPase regulatory subunit 7 (MER030134), eukaryotic translation initiation factor 3 subunit 5 (MER030133), 1FP38 peptidase homologue (MER030132), subfamily M67A non-peptidase homologues (MER191181), subfamily M67A unassigned peptidases (MER191144), granzyme B (Homo sapiens-type) (MER000168), testisin (MER005212), tryptase beta (MER000136), kallikrein-related peptidase 5 (MER005544), conn (MER005881), kallikrein-related peptidase 12 (MER006038), DESC1 peptidase (MER006298), tryptase gamma 1 (MER011036), kallikrein-related peptidase 14 (MER011038), hyaluronan-binding peptidase (MER003612), transmembrane peptidase, serine 4 (MER011104), intestinal serine peptidase (rodent) (MER016130), adrenal secretory serine peptidase (MER003734), tryptase delta 1 (Homo sapiens) (MER005948), matriptase-3 (MER029902), marapsin (MER006119), tryptase-6 (MER006118), ovochymase-1 domain 1 (MER099182), transmembrane peptidase, serine 3 (MER005926), kallikrein-related peptidase 15 (MER000064), Mername-AA031 peptidase (MER014054), DMPRSS13 peptidase (MER014226), Mername-AA038 peptidase (MER062848), Mername-AA204 peptidase (MER029980), cationic trypsin (Homo sapiens-type) (MER000020), elastase-2 (MER000118), mannan-binding lectin-associated serine peptidase-3 (MER031968), cathepsin G (MER000082), myeloblastin (MER000170), granzyme A (MER001379), granzyme M (MER001541), chymase (Homo sapiens-type) (MER000123), tryptase alpha (MER000135), granzyme K (MER001936), granzyme H (MER000166), chymotrypsin B (MER000001), elastase-1 (MER003733), pancreatic endopeptidase E (MER000149), pancreatic elastase II (MER000146), enteropeptidase (MER002068), chymotrypsin C (MER000761), prostasin (MER002460), kallikrein 1 (MER000093), kallikrein-related peptidase 2 (MER000094), kallikrein-related peptidase 3 (MER000115), mesotrypsin (MER000022), complement component Clr-like peptidase (MER016352), complement factor D (MER000130), complement component activated Clr (MER000238), complement component activated Cls (MER000239), complement component C2a (MER000231), complement factor B (MER000229), mannan-binding lectin-associated serine peptidase 1 (MER000244), complement factor I (MER000228), pancreatic endopeptidase E form B (MER000150), pancreatic elastase IIB (MER000147), coagulation factor Xlla (MER000187), plasma kallikrein (MER000203) coagulation factor Xia (MER000210), coagulation factor IXa (MER000216), coagulation factor Vila (MER000215), coagulation factor Xa (MER000212), thrombin (MER000188), protein C (activated) (MER000222), acrosin (MER000078), hepsin (MER000156), hepatocyte growth factor activator (MER000186), mannan-binding lectin-associated serine peptidase 2 (MER002758), u-plasminogen activator (MER000195), t-plasminogen activator (MER000192), plasmin (MER000175), kallikrein-related peptidase 6 (MER002580), neurotrypsin (MER004171), kallikrein-related peptidase 8 (MER005400), kallikrein-related peptidase 10 (MER003645), epitheliasin (MER003736), kallikrein-related peptidase 4 (MER005266), prosemin (MER004214), chymopasin (MER001503), kallikrein-related peptidase 11 (MER004861), kallikrein-related peptidase 11 (MER216142), trypsin-2 type A (MER000021), HtrA1 peptidase (Homo sapiens-type) (MER002577), HtrA2 peptidase (MER208413), HtrA2 peptidase (MER004093), HtrA3 peptidase (MER014795), HtrA4 peptidase (MER016351), Tysndl peptidase (MER050461), DMPRSS12 peptidase (MER017085), HAT-like putative peptidase 2 (MER021884), trypsin C (MER021898), kallikrein-related peptidase 7 (MER002001), matriptase (MER003735), kallikrein-related peptidase 13 (MER005269), kallikrein-related peptidase 9 (MER005270), matriptase-2 (MER005278), umbelical vein peptidase (MER005421), LCLP peptidase (MER001900), spinesin (MER014385), marapsin-2 (MER021929), complement factor D-like putative peptidase (MER056164), ovochymase-2 (MER022410), HAT-like 4 peptidase (MER044589), ovochymase 1 domain 1 (MER022412), epidermis-specific SP-like putative peptidase (MER029900), testis serine peptidase 5 (MER029901), Mername-AA258 peptidase (MER000285), polyserase-IA unit 1 (MER030879), polyserase-IA unit 2 (MER030880), testis serine peptidase 2 (human-type) (MER033187), hypothetical acrosin-like peptidase (Homo sapiens) (MER033253), HAT-like 5 peptidase (MER028215), polyserase-3 unit 1 (MER061763), polyserase-3 unit 2 (MER061748), peptidase similar to tryptophan/serine protease (MER056263), polyserase-2 unit 1 (MER061777), Memame-AA123 peptidase (MER021930), HAT-like 2 peptidase (MER099184), hCG2041 452-like protein (MER099172), hCG22067 (Homo sapiens) (MER099169), brain-rescue-factor-1 (Homo sapiens) (MER098873), hCG204H08 (Homo sapiens) (MER099173), polyserase-2 unit 2 (MER061760), polyserase-2 unit 3 (MER065694), Mername-AA201 (peptidase homologue) MER099175, secreted trypsin-like serine peptidase homologue (MER030000), polyserase-1A unit 3 (MER029880), azurocidin (MER000119), haptoglobin-1 (MER000233), haptoglobin-related protein (MER000235), macrophage-stimulating protein (MER001546), hepatocyte growth factor (MER000185), protein Z (MER000227), TESP1 protein (MER047214), LOC136242 protein (MER016132), plasma kallikrein-like protein 4 (MERO 16346), PRSS35 protein (MER016350), DKFZp586H2123-like protein (MER066474), apolipoprotein (MER000183), psi-KLKI pseudogene (Homo sapiens) (MER033287), tryptase pseudogene I (MER015077), tryptase pseudogene II (MER015078), tryptase pseudogene III (MER015079), subfamily S1A unassigned peptidases (MER216982), subfamily S1A unassigned peptidases (MER216148), amidophosphoribosyltransferase precursor (MER003314), glutamine-fructose-6-phosphate transaminase 1 (MER003322), glutamine:fructose-6-phosphate amidotransferase (MER012158), Mername-AA144 protein (MER021319), asparagine synthetase (MER033254), family C44 non-peptidase homologues (MER159286), family C44 unassigned peptidases (MER185625) family C44 unassigned peptidases (MER185626), secernin 1 (MER045376), secernin 2 (MER064573), secernin 3 (MER064582), acid ceramidase precursor (MER100794), N-acylethanolamine acid amidase precursor (MER141667), proteasome catalytic subunit 1 (MER000556), proteasome catalytic subunit 2 (MER002625), proteasome catalytic subunit 3 (MER002149), proteasome catalytic subunit 1i (MER000552), proteasome catalytic subunit 2i (MER001515), proteasome catalytic subunit 3i (MER000555), proteasome catalytic subunit 5t (MER026203), protein serine kinase cl7 (MER026497), proteasome subunit alpha 6 (MER000557), proteasome subunit alpha 2 (MER000550), proteasome subunit alpha 4 (MER000554), proteasome subunit alpha 7 (MER033250), proteasome subunit alpha 5 (MER000558), proteasome subunit alpha 1 (MER000549), proteasome subunit alpha 3 (MER000553), proteasome subunit XAPC7 (MER004372), proteasome subunit beta 3 (MER001710), proteasome subunit beta 2 (MER002676), proteasome subunit beta 1 (MER000551), proteasome subunit beta 4 (MER001711), Mername-AA230 peptidase homologue (Homo sapiens) (MER047329), Memame-AA23 1 pseudogene (Homo sapiens) (MER047172), Mername-AA232 pseudogene (Homo sapiens) (MER047316), glycosylasparaginase precursor (MER003299), isoaspartyl dipeptidase (threonine type) (MER031622), taspase-1 (MERO 16969), gamma-glutamyltransferase 5 (mammalian-type) (MEROO 1977), gamma-glutamyltransferase 1 (mammalian-type) (MEROO 1629), gamma-glutamyltransferase 2 (Homo sapiens) (MER001976), gamma-glutamyltransferase-like protein 4 (MER002721), gamma-glutamyltransferase-like protein 3 (MERO 16970), similar to gamma-glutamyltransferase 1 precursor (Homo sapiens) (MER026204), similar to gamma-glutamyltransferase 1 precursor (Homo sapiens) (MER026205), Memame-AA211 putative peptidase (MER026207), gamma-glutamyltransferase 6 (MER159283), gamma-glutamyl transpeptidase homologue (chromosome 2, Homo sapiens) (MER037241), polycystin-1 (MER126824), KIAA1879 protein (MER159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647) DJ-1 putative peptidase (MER003390), Mername-AA100 putative peptidase (MER014802), Memame-AAlOl non-peptidase homologue (MER014803), KTAA0361 protein (Homo sapiens-type) (MER042827), F1 134283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER122199), latrophilin-1 (MER126380), latrophilin 3 (MER124612), protocadherin Flamingo 2 (MER124239), ETL protein (MER126267), G protein-coupled receptor 112 (MER126114), seven transmembrane helix receptor (MER125448), Gprl 14 protein (MER159320), GPR126 vascular inducible G protein-coupled receptor (MER140015), GPR125 (Homo sapiens)-type protein (MER159279), GPR116 (Homo sapiens)-type G-protein coupled receptor (MER159280), GPR128 (Homo sapiens)-type G-protein coupled receptor (MER162015), GPR133 (Homo sapiens)-type protein (MER159334), GPR110 G-protein coupled receptor (MER159277), GPR97 protein (MER159322), KPG 006 protein (MER161773), KPG 008 protein (MER161835), KPG 009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Mername-AA195 putative peptidase (MER017367), Memame-AA196 putative peptidase (MER017368), Memame-AA197 putative peptidase (MER017371), Cl4orf29 protein (MER033244), hypothetical protein (MER033245), hypothetical esterase/lipase/thioesterase (MER047309), protein bat5 (MER037840), hypothetical protein flj40219 (MER033212), hypothetical protein flj 37464 (MER033240), hypothetical protein flj33678 (MER033241), dipeptidylpeptidase homologue DPP6 (MER000403), dipeptidylpeptidase homologue DPP 10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase Dl (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MERO 10960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc3285744ike protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccgl-interacting factor b (MER210738), glycosylasparaginase precursor (MER003299), isoaspartyl dipeptidase (threonine type) (MER031622), taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721), gamma-glutamyltransferase-like protein 3 (MERO 16970), similar to gamma-glutamyltransferase 1 precursor (Homo sapiens) (MER026204), similar to gamma-glutamyltransferase 1 precursor (Homo sapiens) (MER026205), Mername-AA21 1 putative peptidase (MER026207), gamma-glutamyltransferase 6 (MER159283), gamma-glutamyl transpeptidase homologue (chromosome 2, Homo sapiens) (MER037241), polycystin-1 (MER126824), KIAA1879 protein (MER159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647), DJ-1 putative peptidase (MER003390), Memame-AAlOO putative peptidase (MER014802), Memame-AAlOl non-peptidase homologue (MER014803), KIAA0361 protein (Homo sapiens-type) (MER042827), FLJ34283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non-peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER122199), latrophilin-1 (MER126380), latrophilin 3 (MER124612), protocadherin Flamingo 2 (MER124239), ETL protein (MER126267), G protein-coupled receptor 112 (MER126114), seven transmembrane helix receptor (MER125448), Gprl 14 protein (MER159320), GPR126 vascular inducible G protein-coupled receptor (MER140015), GPR125 (Homo sapiens)-type protein (MER159279), GPR116 (Homo sapiens)-type G-protein coupled receptor (MER159280), GPR128 (Homo sapiens)-type G-protein coupled receptor (MER162015), GPR133 (Homo sapiens)-type protein (MER159334) GPR110 G-protein coupled receptor (MER159277), GPR97 protein (MER159322), KPG 006 protein (MER161773) KPG 008 protein (MER161835), KPG 009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MFJC1 self-cleaving mucin (MER074260), dystroglycan (MER054741), proprotein convertase 9 (MER022416), site-1 peptidase (MEROO 1948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MERO 17240), Mername-AA194 putative peptidase (MERO 17353), Memame-AA195 putative peptidase (MER017367), Mername-AA196 putative peptidase (MER017368), Mername-AA197 putative peptidase (MER017371), Cl4orf29 protein (MER033244), hypothetical protein (MER033245), hypothetical esterase/lipase/thioesterase (MER047309), protein bat5 (MER037840), hypothetical protein flj40219 (MER033212), hypothetical protein flj 37464 (MER033240), hypothetical protein flj33678 (MER033241), dipeptidylpeptidase homologue DPP6 (MER000403), dipeptidylpeptidase homologue DPP 10 (MER005988), protein similar to Mus musculus chromosome 20 open reading frame 135 (MER037845), kynurenine formamidase (MER046020), thyroglobulin precursor (MERO 11604), acetylcholinesterase (MER033188), cholinesterase (MER033198), carboxylesterase Dl (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA13634ike protein (MER033242), hormone-sensitive lipase (MER033274), neuroligin 1 (MER033280), neuroligin 2 (MER033283), family S9 non peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MERO 10960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccgl-interacting factor b (MER210738).

In some embodiments, the SRS is a peptide moiety of up to 15 amino acids in length. In some embodiments, the SRS is cleaved by a protease co-localized with the target of the cell binding moiety in a tissue, and the protease cleaves the SRS in the therapeutic conjugate when the therapeutic conjugate is exposed to the protease. In some embodiments, the protease is not active or is significantly less active in tissues that do not significantly express the cell surface feature. In some embodiments, the protease is not active or is significantly less active in healthy, e.g., non-diseased tissues.

In some embodiments, the SRS is cleaved by a protease selected from the following:
ADAMS or ADAMTS, e g. ADAM8, ADAM9, ADAM10, ADAM12, ADAM15, ADAM 17/T ACE, ADAMDEC1, ADAMTS 1, ADAMTS4 or ADAMTS5;
Aspartate proteases, e.g., BACE or Renin;
Aspartic cathepsins (to the extent upregulated or released by cell lysis in the extracellular space), e.g., Cathepsin D or Cathepsin E;
Caspases (to the extent upregulated or released by cell lysis in the extracellular space), e.g., Caspase 1, Caspase 2, Caspase 3, Caspase 4, Caspase 5, Caspase 6, Caspase 7, Caspase 8, Caspase 9, Caspase 10 or Caspase 14;
Cysteine cathepsins, e.g., Cathepsin B, Cathepsin C, Cathepsin K, Cathepsin L, Cathepsin S, Cathepsin V/L2, Cathepsin X/Z/P;
Cysteine proteinases, e.g., Cruzipain, Legumain or Otubain-2;
KLKs, e.g., KLK4, KLK5, KLK6, KLK7, KLK8, KLK10, KLK11, KLK13 or KLK14;
Metallo-proteinases, e.g., Meprin, Neprilysin, PSMA or BMP-1;
MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27;
Serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrAl, Human Neutrophil Elastase, Lactoferrin, Marapsin, NS3/4A, PACE4, Plasmin, PSA, tPA, Thrombin, Tryptase or uPA; and/or
Type II Transmembrane Serine Proteases (TTSPs), e.g., DESC1, DPP-4, Hepsin, Matriptase-2, MT-SPl/Matriptase, DMPRSS2, DMPRSS3, DMPRSS4.

For example, suitable SRSs that can be included binder-drug conjugate, i.e., SRS is peptide moiety selected from the group consisting of: TGRGPSWV (SEQ ID NO: 179), SARGPSRW (SEQ ID NO: 180), TARGPSFK (SEQ ID NO: 181), LSGRSDNH (SEQ ID NO: 182), GGWHTGRN (SEQ ID NO: 183), HTGRSGAL (SEQ ID NO: 184), PLTGRSGG (SEQ ID NO: 185), AARGPAIH (SEQ ID NO: 186), RGPAFNPM (SEQ ID NO: 187), SSRGPAYL (SEQ ID NO: 188), RGPATPIM (SEQ ID NO: 189), RGPA (SEQ ID NO: 190), GGQPSGMWGW (SEQ ID NO: 191), FPRPLGITGL (SEQ ID NO: 192), VHMPLGFLGP (SEQ ID NO: 193), SPLTGRSG (SEQ ID NO: 194), SAGFSLPA (SEQ ID NO: 195), LAPLGLQRR (SEQ ID NO: 196), SGGPLGVR (SEQ ID NO: 197), PLGL (SEQ ID NO: 198), GPRSFGL (SEQ ID NO: 199), and GPRSFG (SEQ ID NO: 200).

In some embodiments, the SRS is a substrate for an MMP, such as a sequence selected from the group consisting of ISSGLLSS (SEQ ID NO: 201), QNQALRMA (SEQ ID NO: 202), AQNLLGMV (SEQ ID NO: 203), STFPFGMF (SEQ ID NO: 204), PVGYTSSL (SEQ ID NO: 205), DWLYWPGI (SEQ ID NO: 206), MIAPVAYR (SEQ ID NO: 207), RPSPMWAY (SEQ ID NO: 208), WATPRPMR (SEQ ID NO: 209), FRLLDWQW (SEQ ID NO: 210), LKAAPRWA (SEQ ID NO: 211), GPSHLVLT (SEQ ID NO: 212), LPGGLSPW (SEQ ID NO: 213), MGLFSEAG (SEQ ID NO: 214), SPLPLRVP (SEQ ID NO: 215), RMHLRSLG (SEQ ID NO: 216), LAAPLGLL (SEQ ID NO: 217), AVGLLAPP (SEQ ID NO: 218), LLAPSHRA (SEQ ID NO: 219), PAGLWLDP (SEQ ID NO: 220), and ISSGLSS (SEQ ID NO: 221).

In some embodiments, the SRS is a substrate for an MMP, such as a sequence selected from the group consisting of ISSGLSS (SEQ ID NO: 221), QNQALRMA (SEQ ID NO: 202), AQNLLGMV (SEQ ID NO: 203), STFPFGMF (SEQ ID NO: 204), PVGYTSSL (SEQ ID NO: 205), DWLYWPGI (SEQ ID NO: 206), ISSGLLSS (SEQ ID NO: 201), LKAAPRWA (SEQ ID NO: 211), GPSHLVLT (SEQ ID NO: 212), LPGGLSPW (SEQ ID NO: 213), MGLFSEAG (SEQ ID NO: 214), SPLPLRVP (SEQ ID NO: 215), RMHLRSLG (SEQ ID NO: 216), LAAPLGLL (SEQ ID NO: 217), AVGLLAPP (SEQ ID NO: 218), LLAPSHRA (SEQ ID NO: 219), and PAGLWLDP (SEQ ID NO: 220).

In some embodiments, the SRS is a substrate for thrombin, such as GPRSFGL (SEQ ID NO: 199) or GPRSFG (SEQ ID NO: 200).

Spacers

In some embodiments, a therapeutic conjugate comprises a spacer or bond (L) between the half-life extension moiety and the substrate recognition sequence (SRS) cleavable by the enzyme, e.g., present in a tumor microenvironment.

The spacer may be any molecule, for example, one or more nucleotides, amino acids, chemical functional groups. In some embodiments, the spacer is a peptide linker (e.g., two or more amino acids). Spacers should not adversely affect the expression, secretion, or bioactivity of the polypeptides. In some embodiments, spacers are not antigenic and do not elicit an immune response. An immune response includes a response from the innate immune system and/or the adaptive immune system. Thus, an immune response may be a cell-mediate response and/or a humoral immune response. The immune response may be, for example, a T cell response, a B cell response, a natural killer (NK) cell response, a monocyte response, and/or a macrophage response. Other cell response are contemplated herein. In some embodiments, linkers are non-protein-coding.

In some embodiments, L¹ is a hydrocarbon (straight chain or cyclic) such as 6-maleimidocaproyl, maleimidopropanoyl and maleimidom ethyl cyclohexane-1-carboxylate, or L¹ is N-Succinimidyl 4-(2-pyridylthio) pentanoate, N-Succinimidyl 4-(N-maleimidomethyl) cyclohexane-1 carboxylate, N-Succinimidyl (4-iodo-acetyl) aminobenzoate.

In some embodiments, L¹ is a polyether such as a poly(ethylene glycol) or other hydrophilic linker. For instance, where the CBM includes a thiol (such as a cysteine residue), L1 can be a polyethylene glycol) coupled to the thiol group through a maleimide moiety. Non-limiting examples of linkers for use in accordance with the present disclosure are described in International Publication No. WO 2019/236567, published Dec. 12, 2019, incorporated by reference herein.

Self-Immolative Linkers

In some embodiments, a therapeutic conjugate comprises a self-immolative linker (L²) between the substrate recognition sequence (SRS) for the enzyme and the drug moiety, such as represented in the formula

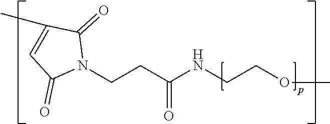

wherein, p represents an integer from 1 to 100, preferably 6 to 50, more preferably 6 to 12. In other embodiments, where the CBM includes a thiol and L¹ is a hydrocarbon moiety coupled to the thiol group through a maleimide moiety, L¹ can be represented in the formula

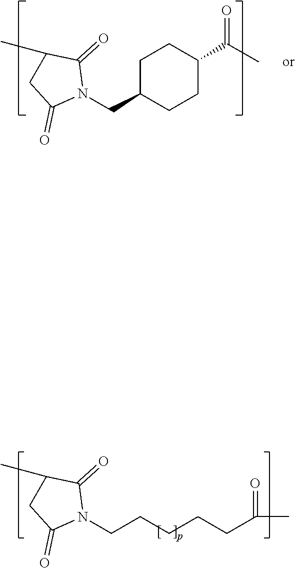

wherein, p represent an integer from 1 to 20, preferably 1 to 4. A self-immolative moiety may be defined as a bifunctional chemical group that is capable of covalently linking together two spaced chemical moieties into a normally stable molecule, releasing one of the spaced chemical moieties from the molecule by means of enzymatic cleavage; and following enzymatic cleavage, spontaneously cleaving from the remainder of the bifunctional chemical group to release the other of said spaced chemical moieties. Therefore, in some embodiments, the self-immolative moiety is covalently linked at one of its ends, directly or indirectly through a spacer unit, to the ligand by an amide bond and covalently linked at its other end to a chemical reactive site (functional group) pending from the drug moiety. The derivatization of the drug moiety with the self-immolative moiety may render the drug less pharmacologically active (e.g. less toxic) or not active at all until the drug is cleaved.

A therapeutic conjugate is generally stable in circulation, or at least that should be the case in the absence of an enzyme capable of cleaving the amide bond between the substrate recognition sequence (enzyme-cleavable linker) and the self-immolative moiety. Upon exposure of a therapeutic conjugate to a suitable enzyme, the amide bond is cleaved initiating a spontaneous self-immolative reaction resulting in the cleavage of the bond covalently linking the self-immolative moiety to the drug moiety, to thereby effect release of the free drug moiety in its underivatized or pharmacologically active form. The self-immolative moiety in conjugates either incorporate one or more heteroatoms and thereby provides improved solubility, improves the rate of cleavage and decreases propensity for aggregation of the conjugate.

In some embodiments, L² is a benzyl oxy carbonyl group. In other embodiments, the self-immolative linker L² is —NH—(CH₂)₄—C(═O)— or NH—(CH₂)₃—C(═O)—. In yet other embodiments, the self-immolative linker L² is p-aminobenzyloxycarbonyl (PABC). In still other embodiments, the self-immolative linker L² is 2,4-bis(hydroxymeihyl)aniline.

The therapeutic conjugates of the present disclosure can employ a heterocyclic self-immolative moiety covalently linked to the therapeutic moiety and the cleavable substrate recognition sequence. A self-immolative moiety may be defined as a bifunctional chemical group which is capable of covalently linking together two spaced chemical moieties into a normally stable molecule, releasing one of said spaced chemical moieties from the molecule by means of enzymatic cleavage; and following said enzymatic cleavage, spontaneously cleaving from the remainder of the bifunctional chemical group to release the other of said spaced chemical moieties. In accordance with the present present disclosure, the self-immolative moiety may be covalently linked at one of its ends, directly or indirectly through a spacer unit, to the ligand by an amide bond and covalently linked at its other end to a chemical reactive site (functional group) pending from the drug. The derivatization of the therapeutic moiety with the self-immolative moiety may render the drug less pharmacologically active (e.g. less toxic) or not active at all until the drug is cleaved.

The therapeutic conjugate is generally stable in circulation, or at least that should be the case in the absence of an enzyme capable of cleaving the amide bond between the substrate recognition sequence and the self-immolative moiety. However, upon exposure of the therapeutic conjugate to a suitable enzyme, the amide bond is cleaved initiating a spontaneous self-immolative reaction resulting in the cleavage of the bond covalently linking the self-immolative moiety to the drug, to thereby effect release of the free therapeutic moiety in its underivatized or pharmacologically active form.

The self-immolative moiety in conjugates of the present disclosure, in some embodiments, either incorporate one or more heteroatoms and thereby provides improved solubility, improves the rate of cleavage and decreases propensity for aggregation of the conjugate. These improvements of the heterocyclic self-immolative linker constructs of the present disclosure over non-heterocyclic, PAB-type linkers may result in surprising and unexpected biological properties such as increased efficacy, decreased toxicity, and more desirable pharmacokinetics.

In some embodiments, L² is a benzyloxycarbonyl group.

In some embodiments, L² is

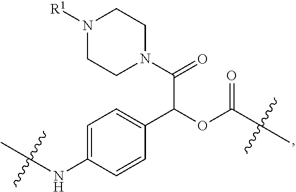

wherein R¹ is hydrogen, unsubstituted or substituted C₁₋₃ alkyl, or unsubstituted or substituted heterocyclyl. In some embodiments, R¹ is hydrogen. In some instances, R¹ is methyl.

In some embodiments, L² is selected from

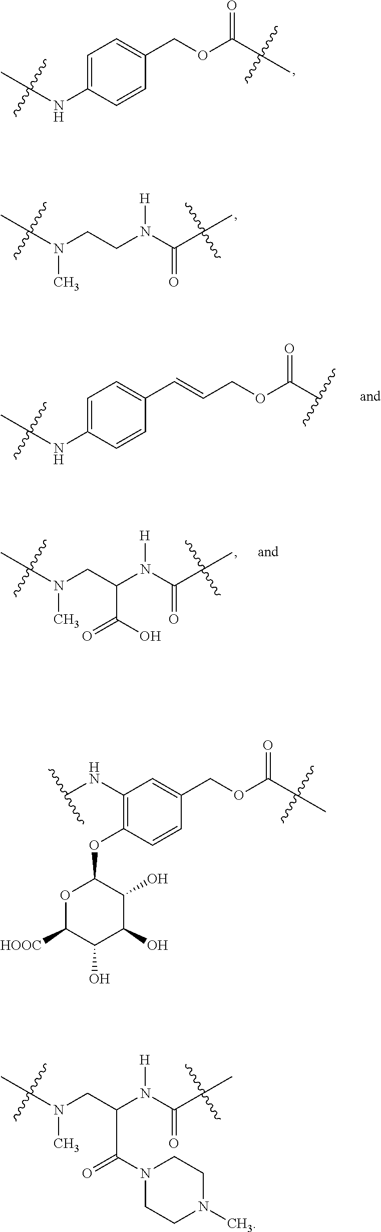

In some embodiments, the self-immolative moiety L2 is selected from

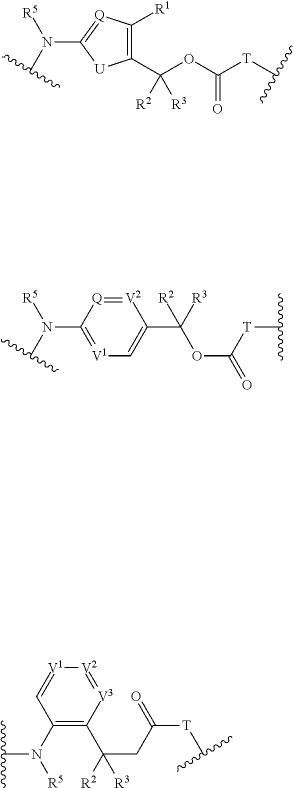

wherein
U is O, S or NR⁶;
Q is CR⁴ or N;
V¹, V² and V³ are independently CR⁴ or N provided that for formula II and III at least one of Q, V¹ and V² is N;
T is NH, NR⁶, O or S pending from said therapeutic moiety;
R¹, R², R³ and R⁴ are independently selected from H, F, Cl, Br, I, OH, —N(R⁵)₂, —N(R⁵)₃⁺, C₁-C₈ alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and
R⁵ and R⁶ are independently selected from H, C₁-C₈ alkyl, C₁-C₈ substituted alkyl, C₂-C₈ alkenyl, C₂-C₈ substituted alkenyl, C₂-C₈alkynyl, C₂-C₈ substituted alkynyl, C₆-C₂₀ aryl, C₆-C₂₀ substituted aryl, C₁-C₂₀ heterocycle, and C₁-C₂₀ substituted heterocycle;
where C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate.

It will be understood that when T is NH, it is derived from a primary amine (—NH₂) pending from the therapeutic moiety (prior to coupling to the self-immolative moiety) and when T is N, it is derived from a secondary amine (—NH—) from the therapeutic moiety (prior to coupling to the self-immolative moiety). Similarly, when T is O or S, it is derived from a hydroxyl (—OH) or sulfhydryl (—SH) group respectively pending from the therapeutic moiety prior to coupling to the self-immolative moiety.

In some embodiments, the self-immolative linker L² is —NH—(CH₂)₄—C(═O) or —NH—(CH₂)₃—C(═O)—.

In some embodiments, the self-immolative linker L² is p-aminobenzyloxycarbonyl (PABC).

In some embodiments, the self-immolative linker L² is 2,4-bis(hydroxymethyl)aniline.

Other examples of self-immolative linkers that are readily adapted for use in therapeutic conjugates described herein are taught in, for example, U.S. Pat. No. 7,754,681; WO 2012/074693A1; U.S. Pat. No. 9,089,614; EP 1,732,607; WO 2015/038426A1 (all of which are incorporated by reference); Walther et al. “Prodrugs in medicinal chemistry and enzyme prodrug therapies” Adv Drug Deliv Rev. 2017 Sep. 1; 118:65-77; and Tranoy-Opalinski et al. “Design of self-immolative linkers for tumor-activated prodrug therapy”, Anticancer Agents Med Chem. 2008 August; 8(6):618-37; the teachings of each of which are incorporated by reference herein.

Yet other non-limiting examples of self-immolative linkers for use in accordance with the present disclosure are described in International Publication No. WO 2019/236567, published Dec. 12, 2019, incorporated by reference herein.

### Drug Moieties

In some embodiments, the drug moiety (-DM) is an immune inducing agent, such as an agent that induces inflammation resulting in activation of innate and/or adaptive immune responses.

In some embodiments, the drug moiety (-DM) is a cytotoxic agent, i.e., which when released from the therapeutic conjugate causes cell death of the target cells at the concentration at which the therapeutic conjugate is administered.

In some embodiments, the drug moiety (-DM) is a cytostatic agent, i.e., which when released from the therapeutic conjugate causes mitotic arrest or quiescence of the target cells at the concentration at which the therapeutic conjugate is administered.

In some embodiments, the drug moiety (-DM) is an epigenetic agent, i.e., which when released from the therapeutic conjugate causes epigenetic alteration of the target cells at the concentration at which the therapeutic conjugate is administered, which may, for example, result in differentiation (or dedifferentiation) of the cell to another cellular phenotype.

In some embodiments, the drug moiety DM is represented by the general formula

—RBM [symbol] Z)ₚ

wherein RBM is a receptor binding moiety, Z is cytotoxic, cytostatic or epigenetic moiety or a radioisotope containing moiety, and p is 0 (Z is absent) or an integer from 1 to 8. In some embodiments, p is 1.

In some embodiments, the drug moiety DM is represented by the general formula

—RBM-L³-Z

wherein RBM is a receptor binding moiety, Z is cytotoxic, cytostatic or epigenetic moiety or a radioisotope containing moiety, and L³ is a bond or a cleavable or non-cleavable linker. For instance, L³ can be a linker that is acid labile or enzyme sensitive (such as includes a cathepsin cleavage site) such that Z is released intracellularly on internalization of the moiety —RBM-L³-Z through cell binding dependent on the receptor binding moiety RBM.

To further illustrate, the receptor binding moiety (RBM) can be a ligand for a receptor, such as ligand that when released from the therapeutic conjugate is able to bind to an extracellular ligand binding domain of a cell surface receptor. Exemplary receptor ligands include somatostatin, cholecystokinin-2 (CCK2), folate (folic acid), bombesin, gastrin-releasing peptide, calcitonin, oxytocin, EGF, α-melanocyte-stimulating hormone, minigastrin, neurotensin, substance P, glucagon-like peptide 1, neuropeptide Y and analogs of those ligands. The receptor ligand can itself have pharmacological activity in and of itself or can be used to deliver a conjugated drug moiety, toxin or radioisotope to (and preferably into) the cell expressing the receptor.

Additional examples include embodiments where the receptor binding moiety is a ligand or receptor agonist selected from the group consisting of: folate derivatives (proteins that bind to folate receptors and are overexpressed in ovarian cancer and other malignancies) (Low, P S et al 2008, acc. chem. res. 41, 120-9); glutamate urea derivatives (binding to prostate specific membrane antigen, surface markers for prostate Cancer cells) (Hillier, S M et al, 2009, Cancer res. 69, 6932-40); somatostatin (also known as Growth Hormone Inhibiting Hormone (GHIH) or growth hormone release inhibiting factor (SRIF) or growth hormone release inhibiting hormone) and its homologues, such as octreotide (Sandostatin) and lanreotide (Somatuline) (particularly for neuroendocrine tumors, GH-producing pituitary adenomas, paragangliomas, non-functional pituitary adenomas, pheochromocytomas (Ginj, M., et al, 2006, Proc. Natl. Acad. Sci. USA 103, 16436-41), the somatostatin receptor subtypes in the following tumors (sst1, sst2, sst3, sst4 and sst5), GH-secreting pituitary adenomas (Reubi J C, L and olt, AM1984J. Clin. Endocrinol Metab 59: 1148-51; Re J C, L and olt A M 1987 Clin. Endocrinol Metab 65: 65. C.73; Moyse E, et al, J Clin Endocrinol Metab 61: 98-103), gastroenteropancreatic tumors (Reubi J C, et al, 1987J Clin Endocrinol Metab 65: 1127-34; Reubi, J. C, et al, 1990 Cancer Res 50: 5969-77), pheochromocytoma (Epel-baum J, et al, 1995J Clin Endocrinol Metab 80: 1837-44; Reubi J C, et al, 1992J Clin Endocrinol Metab 74: 1082-9), neuroblastoma (Prevost G, 1996 neoendocrinolology 63: 188) 197; Mortel, Mill. L, et al, 1994 Amob J P102: 752-752), thyroid medullary carcinoma (Reubi, J. C, et al, 1991 Lab 64: 7J, 1987J, 1987, et al, 1987J, 1987J, mouse colon receptor for colon Cancer, mouse colon receptor, mouse colon receptor, mouse colon receptor, mouse colon receptor, mouse colon receptor, mouse colon receptor, mouse colon receptor, mouse The somatic subtype (CCK); bombesin (Pyr-Gln-Arg-Leu-Gly-Asn-Gln-Trp-Ala-Val-Gly-His-Leu-Met-NH)2) (SEQ ID NO: 143) Or Gastrin Releasing Peptide (GRP) and its receptor subclasses (BB1, GRP receptor subclasses (BB2), BB3 and BB4) (Ohlsson, b., et al, 1999, Sc and j.gastroenterology 34(12) 1224-9; weber, H C, 2009, cur. opin. endocri. dib. obesity 16(1): 66-71, Gonzalez N, et al, 2008, cur. opin. endocri. dib. obesity 15(1), 58-64); neurotensin receptors and their receptor subtypes (NTR1, NTR2, NTR 3); substance P receptors and their receptor subtypes (e.g., NK1 receptor for glial tumors, Hennig I M, et al 1995 int. J. cancer 61, 786-cake 792); neuropeptide Y (npy) receptor and its receptor subtype (Y1-Y6); homing peptides include RGD (Arg-Gly-Asp), NGR (Asn-Gly-Arg), dimeric and multimeric cyclic RGD peptides (e.g., cRGDfV) (Laakkonen P, Vuorine K. 2010, Integr Biol (Camb). sub.2 (7-8): 326-337; Chen K, Chen X. 2011, Theranostics. 1: 189-200; Garanger E, et al, Anti-Cancer Agents Med. 7(5): 552-558; Kerr, J S et al, Antincerner Research, 19(2A), 959-968; Thumshirn, G, et al, 2003 Chem. Eur. J. 9, 7-2725), and TAASGVRSMH (SEQ ID NO: 144) or LTLRWVGLMS (SEQ ID NO: 145) (chondroitin sulfate proteoglycan NG2 receptor) and F3 (nucleolin receptor binding peptides) for cell surface expression (RG receptor) (amino acid receptor 31, BTE-Gly-26, WO 31, 92-26, Biodamen K. 31, Biodamen K. 326; And K. 32, Biodamn. 31, Biodamn K. 32; And K. 32, Biodamn. 9, JP 31, 9, K, 9, K, 9, No. 35, No. 20, No. 35, No., proc. nat. acad. sci. usa 99(11), 7444-9); cell Penetrating Peptides (CPPs) (Nakase I, et al, 2012, J. Control Release. 159(2), 181-188); peptide hormones, such as agonists and antagonists of Luteinizing Hormone Releasing Hormone (LHRH), gonadotropin releasing hormone (GnRH) agonists, acting by targeting Follicle Stimulating Hormone (FSH) and Luteinizing Hormone (LH) as well as testosterone production, such as buserelin (Pyr-His-Trp-Ser-Tyr-D-Ser (OtBu)-Leu-Arg-Pro-NHEt) (SEQ ID NO: 146), gonadorelin (Pyr-His-Trp-Ser-Tyr-Gly-Leu-Arg-Pro-Gly-NH2) (SEQ ID NO: 147), goserelin (Pyr-His-Trp-Ser-Tyr-D-Ser (OtBu)-Leu-Arg-Pro-AzGly-NH)2) (SEQ ID NO: 148) Himalarelin (Pyr-His-Trp-Ser-Tyr-D-His (N-Bn)-Leu-Arg-Pro-NHEt) (SEQ ID NO: 149), leuprolide (Pyr-His-Trp-Ser-Tyr-D-Leu-Leu-Arg-Pro-NHEt) (SEQ ID NO: 150), nafarelin (Pyr-His-Trp-Ser-Tyr-2 Nal-Leu-Arg-Pro-Gly-NH)2) (SEQ ID NO: 151), Triptorelin (Pyr-His-Trp-Ser-Tyr-D-Trp-Leu-Arg-Pro-Gly-NH)2) (SEQ ID NO: 152) Nafarelin, desloralin, Aberelin (Ac-D-2Nal-D-4-chloroPhe-D-3-(3-pyridol) Ala-Ser-(N-Me) Tyr-D-Asn-Leu-isopyLLys-Pro-DAla-NH2) (SEQ ID NO: 153) Cetrorelix (Ac-D-2Nal-D-4-chloro-Phe-D-3-(3-pyridol) Ala-Ser-Tyr-D-Cit-Leu-Arg-Pro-D-Ala-NH2) (SEQ ID NO: 154), degarelix (Ac-D-2Nal-D-4-chloroPhe-D-3-(3-pyridol) Ala-Ser-4-aminoPhe (L-hydroxyl)-D-4-aminoPhe (carba-moyl)-Leu-isoproylLys-Pro-D-Ala-NH)2) (SEQ ID NO: 155) And galangal (Ac-D-2Nal-D-4-chloroPhe-D-3-(3-pyridol) Ala-Ser-Tyr-D-(N9,N10-diethyl)-homoArg-Leu-(N9,N10-diethyl)-homoArg-Pro-D-Ala-NH2) (SEQ ID NO: 156) (Thundimadathil, J., J. Aminoacids in Urology 3(3): 127-140; Debruyne, F., 2006, Future Oncology,2 (6); 677 696; Schally A. V; Nagy, A.1999 Eur J Endocrinol 141: 1-14; Koppan M, et al 1999 Protate 38: 151-158); Pattern Recognition Receptors (PRRs), such as Toll-like receptors (TLRs), C-type lectins and nodular receptors (NLRs) (Fukata, M. et al, 2009, Semin. 21, Immunol 242-253; Maisoneune euonymus, C. et al, 2014, J. Ach. 56, J.56.56, J.2011., J.56, J.2011.56, J.2011, 19870, 19856, USA, 14, III, V.56, III, V.11, III. Short peptides, GRGDSPK (SEQ ID NO: 157) and cyclic RGD pentapeptides, such as cyclo (RGDfV) (SEQ ID NO: 160) (L1) and its derivatives (cyclo (-N (Me) R-GDfV) SEQ ID NO: 158), cyclo (R-Sar-DfV) SEQ ID NO: 159), cyclo-(RG-N(Me) D-fV) SEQ ID NO: 176), cyclo (RGD-N(Me) fV) SEQ ID NO: 177), cyclo (RGDf-N(Me) V-) (cilengitide)) SEQ ID NO: 178) have high affinity for integrin receptors (Dechantsreiter, M A et al, 1999 J. Med. chem. 42, 3033-40, Goodman, S L, et al, 2002 J. Med. chem. 45, 1045-51).

In some embodiments, the receptor binding moiety binds to a integrin αvβ3, a gastrin-releasing peptide receptor (GRPR), a somatostatin receptor (such as somatostatin receptor subtype 2), a melanocortin receptor, a cholecystokinin-2 receptor, a neuropeptide Y receptor or a neurotensin receptor.

In some embodiments, the receptor binding moiety binds to folate receptor α, and can be a folate receptor ligand, such as folic acid or folic acid analogs (such as etarfolatide, vintafolide, leucovorin and methotrexate).

In some embodiments, the receptor binding moiety binds to somatostatin receptor, and can be somatostatin or a somatostatin analogs, such as octreotate, octreotide or pentetreotide.

In some embodiments, the receptor binding moiety binds to αIIbβ3, and can be an αIIbβ3-targeted ligand, such as RGD or an RGD analog (i.e., dimer or multimeric analog), including illustrative cyclic RGD peptides like cyclo(-Arg-Gly-Asp-D-Phe Val-) [“c(RGDfV)” ](SEQ ID NO: 160), c(RGDfK) (SEQ ID NO: 161), c(RGDfC) (SEQ ID NO: 162), c(RADfC) (SEQ ID NO: 163), c(RADfK) (SEQ ID NO: 164), c(RGDfE) (SEQ ID NO: 165), c(RADfE) (SEQ ID NO: 166), RGDSK (SEQ ID NO: 167), RADSK (SEQ ID NO: 168), RGDS(SEQ ID NO: 169), c(RGDyC) (SEQ ID NO: 170), c(RADyC) (SEQ ID NO: 171), c(RGDyE) (SEQ ID NO: 172), c(RGDyK) (SEQ ID NO: 173), c(RADyK) (SEQ ID NO: 174) and H-E[c(RGDyK)]2 (SEQ ID NO: 175), EMD 12194, DMP728, DMP757 and SK&F107260.

The drug moieties provided herein are acutely toxic. As used herein, a moiety that is “acutely toxic” refers to a moiety that has adverse effects within 14 days of administration to a subject. Acute toxicity is compared to chronic toxicity or cumulative toxicity, in which the build-up of a drug moiety over time results in the drug moiety's toxicity.

The drug moieties provided herein, in some embodiments, induce an innate immune response in vivo. Innate immune responses include cellular responses to exogenous nucleic acids or proteins, typically of viral or bacterial origin, resulting in increased cytokine expression and release and cell death. Therefore, the innate immune response can be measured by the expression or activity level of cytokines (e.g., Type 1 interferons) or the expression levels of interferon-regulated genes (e.g., toll-like receptors). In some embodiments, the innate immune response is measured by the expression or activity level of granulocyte colony-stimulating factor (G-CSF). The response may also be measured by the level of cell death.

In some embodiments, a therapeutic conjugate induces a lower innate immune response compared to the drug moiety alone. For example, in some embodiments, a therapeutic conjugate induces an innate immune response that is at least 10%, 20%, 30%, 40%, 50%, 60%, 70%, 80%, 90%, or 100% lower than the innate immune response induced when the drug moiety is administered alone.

Toxicity may be measured using any method. In some embodiments, toxicity is measured by liver activity. In some embodiments, toxicity is measured by kidney activity. In some embodiments, toxicity is measured by pancreas activity.

In some embodiments, toxicity is measured by assessing circulating liver enzymes such as aspartate transaminase (AST) and/or alanine transaminase (ALT). AST and/or ALT levels at timepoints after the administration of drug moiety may be compared to baseline values obtained prior to administration, for example, to those of control animals that did not receive the drug moiety, or to values known to be associated with normal animals from previous experiments (historical controls) or from literature.

In some embodiments, toxicity is measured by assessing alkaline phosphatase (ALP) levels. ALP levels at timepoints after the administration of a drug moiety may be compared to baseline values obtained prior to administration, for example, to those of control animals that did not receive the drug moiety, or to values known to be associated with normal animals from previous experiments (historical controls) or from literature.

In some embodiments, toxicity is measured by assessing total bilirubin (TBIL) levels. TBIL levels at timepoints after the administration of a drug moiety may be compared to baseline values obtained prior to administration, for example, to those of control animals that did not receive the drug moiety, or to values known to be associated with normal animals from previous experiments (historical controls) or from literature.

In some embodiments, toxicity is measured by assessing albumin levels. Albumin levels at timepoints after the administration of a drug moiety may be compared to baseline values obtained prior to administration, for example, to those of control animals that did not receive the drug moiety, or to values known to be associated with normal animals from previous experiments (historical controls) or from literature.

In some embodiments, toxicity is measured by assessing serum glucose levels. Serum glucose levels at timepoints after the administration of a drug moiety may be compared to baseline values obtained prior to administration, for example, to those of control animals that did not receive the drug moiety, or to values known to be associated with normal animals from previous experiments (historical controls) or from literature.

In some embodiments, toxicity is measured by assessing lactate dehydrogenase (LDH) levels. Lactate dehydrogenase (LDH) levels at timepoints after the administration of a drug moiety may be compared to baseline values obtained prior to administration, for example, to those of control animals that did not receive the drug moiety, or to values known to be associated with normal animals from previous experiments (historical controls) or from literature.

Without wishing to be bound by theory, it is thought that a therapeutic conjugate holds the drug moiety from being active until the enzyme cleaves the linker. In this way, a therapeutic conjugate is not acutely toxic and has a higher therapeutic index (TI) than the free drug moiety. TI is a quantitative measure of the relative safety of the drug moiety, calculated by comparing the amount of the drug moiety (or conjugate) that causes the therapeutic effect to the amount of the drug moiety (or conjugate) that causes toxicity. In some embodiments, a therapeutic conjugate has a therapeutic index (TI) when delivered systemically that is at least 2-fold greater than the systemic delivery of the free drug moiety, for example, at least 5, 10, 20, 30, 40, 50, 100, 250, 500 or even 1000 greater than the systemic delivery of the free drug moiety. In some embodiments, the therapeutic index (TI) of a therapeutic conjugate is at least 5 times greater than the therapeutic index for the free drug moiety when given systemically, for example at least 10, 20, 30, 40, 50, 75 or even 100 times greater.

In this way, drug moieties that were previously ineffective due to dose-limiting toxicities (e.g., talabostat), can be administered at therapeutic doses (e.g., intratumoral concentrations of 75 nM, 80 nM, 85 nM, 90 nM, 95 nM, 100 nM, 125 nM, 150 nM, 175 nM, 200 nM, or more), as described herein.

Drug moieties provided herein may be prodrugs, which are drugs (e.g., small molecules having a molecular weight of 10 kD or lower) that are inert in their prodrug form (e.g., have a reduced risk of systemic toxicities, have reduced cell permeability), but are activated by cleavage of the enzyme-cleavable linker. Upon cleavage, the drug moiety is released into the extracellular space of the diseased tissue (e.g., tumor), where it may cause a local inflammatory response, promote immune cell infiltration, and in some embodiments, induce an innate immune response.

When the diseased tissue is a tumor, the activated drug moiety may also degrade the tumor stroma, and/or kill tumor cells.

In some embodiments, free drug moiety interacts with an intracellular target and the pharmacological effect of the drug moiety is dependent on the free drug moiety being cell permeable, i.e., and able to interact with its intracellular target, whereas when part of the binder-drug conjugate the drug moiety is substantially cell impermeable. For instance, the rate of accumulation of the binder-drug conjugate intracellularly is less than 50% of the rate for the free drug moiety, for example, less than 25%, 10%, 5%, 1% or even less than 0.1% of the rate for the free drug moiety. For instance, the EC50 for the pharmacological effect of the free drug moiety is at least 2 fold less than (more potent than) the binder-drug conjugate, for example, at least 5, 10, 20, 30, 40, 50, 100, 250, 500 or even 1000 less than the binder-drug conjugate.

In some embodiments, the free drug moiety interacts with an extracellular target and the pharmacological effect of the drug moiety is substantially attenuated when covalently linked to L¹. For instance, the EC50 for the pharmacological effect of the free drug moiety is at least 2 fold less than (more potent than) the binder-drug conjugate, for example, at least 5, 10, 20, 30, 40, 50, 100, 250, 500 or even 1000 less than the binder-drug conjugate.

In some embodiments, the binder-drug conjugate has a therapeutic index when delivered systemically that is at least 2-fold greater than the systemic delivery of the free drug moiety, for example, at least 5, 10, 20, 30, 40, 50, 100, 250, 500 or even 1000 greater than the systemic delivery of the free drug moiety.

In some embodiments, the free drug moiety is an immunomodulator—which includes drug moieties acting as immune activating agents and/or inducers of an innate immunity pathway response. In some embodiments, the free drug moiety induces the production of IFN-α. In some embodiments, the free drug moiety induces the production of proinflammatory cytokines. In some embodiments, the free drug moiety induces the production of IL-1β. In some embodiments, the free drug moiety induces the production of IL-18.

In some embodiments, the free drug moiety promotes the expansion and survival of effector cells including NK, γδ T, and CD8+ T cells.

In some embodiments, the free drug moiety is a damage-associated molecular pattern molecule. In some embodiments, the free drug moiety is a pathogen-associated molecular pattern molecule.

In some embodiments, the drug moiety is a toll-like receptor (TLR) agonist, retinoic acid-inducible gene I (RIG-I) agonist, iDASH inhibitor, or STING agonist, as described in more detail below. Other non-limiting examples of drug moieties are provided in WO 2019/236567, incorporated herein by reference.

Toll-Like Receptor Agonists

In some embodiments, the free drug moiety is a Toll-like receptor (TLR) agonist, such as a selected from the group consisting of a TLR1/2 agonist, a TLR2 agonist, a TLR3 agonist, a TLR4 agonist, a TLR5 agonist, a TLR6/2 agonist, a TLR7 agonist, a TLR7/8 agonist, a TLR7/9 agonist, a TLR8 agonist, a TLR9 agonist, and a TLR11 agonist, for example, selected from the group consisting of a TLR3 agonist, a TLR7 agonist, a TLR7/8 agonist, and a TLR9 agonist. Toll-like receptor (TLR) agonists are involved in activating both innate and adaptive immune responses. Most TLR agonists activate professional antigen-presenting cells (APCs), such as dendritic cells and interact with T cells. As TLRs are central to innate immunity, TLR agonists have been used to enhance TLR activity and, relatedly, activate the innate immune response. TLR agonists are short, synthetic stretches of single-stranded DNA typically comprising a CpG dinucleotide motif.

Examples of toll-like receptor (TLR) agonists include, but are not limited to, TLR1/2 agonists, TLR2 agonists, TLR3 agonists (e.g., PolyTC), TLR4 agonists (e.g., S-type lipopolysaccharide, paclitaxel, lipid A, and monophosphoryl lipid A), TLR5 agonists (e.g., flagellin), TLR6/2 agonists (e.g., MALP-2), TLR7 agonist, TLR7/8 agonists (e.g., gardiquimod, imiquimod, loxoribine, and resiquimod (R848)), TLR7/9 agonists (e.g., hydroxychloroquine sulfate), TLR8 agonists (e.g., motolimod (VTX-2337)), TLR9 agonists (e.g., CpG-ODN), and TLR11 agonists (e.g., profilin).

Examples of TLR agonists that can be used as the drug moiety in the conjugates of include S-27609, CL307, UC-IV150, imiquimod, gardiquimod, resiquimod, motolimod, VTS-1463GS-9620, GSK2245035, DMX-101, DMX-201, DMX-202, isatoribine, AZD8848, MEDI9197, 3M-051, 3M-852, 3M-052, 3M-854A, S-34240, KU34B, or CL663, or as appropriate, analogs thereof with appropriate functional groups for directed linkage and release from the substrate recognition sequence or by linkage to a self-immolative linker.

Examples of agonists of TLRs, particularly TLR7 agonists, TLR8 agonists and TLR7/8 agonists include: isatoribine, loxoribine, bropirimine, imiquimod, resiquimod, gardiquimod, CL097, 3M-002, R848, SM360320, CL264, 3M-003, IMDQ, PF-04878691, motolimod (VTX-2337), and GSK2245035. See, e.g., International Publication No. WO 2019/236567, published Dec. 12, 2019, for chemical structures of the foregoing TLR agonists.

In some embodiments, the drug moiety is a TRL7/8 agonist represented in the general formula

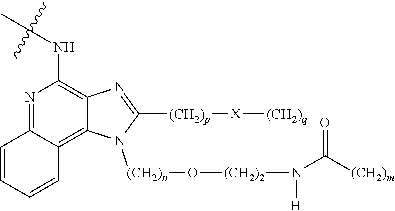

wherein
X is CH₂, O, S or N, for example, CH₂, O or N;
n is 0 (direct bond from N to O), or an integer from 1 to 5, for example 1 or 2; z is an integer from 1 to 5;
m is an integer from 1 to 20, for example from 1 to 16;
p is 0 (direct bond from ring to X), or an integer from 1 to 5, for example 1 or 2; and q is an integer from 1 to 5, for example 1 or 2. For instance, the TRL agonist is a TRL7/8 agonist such as one of:

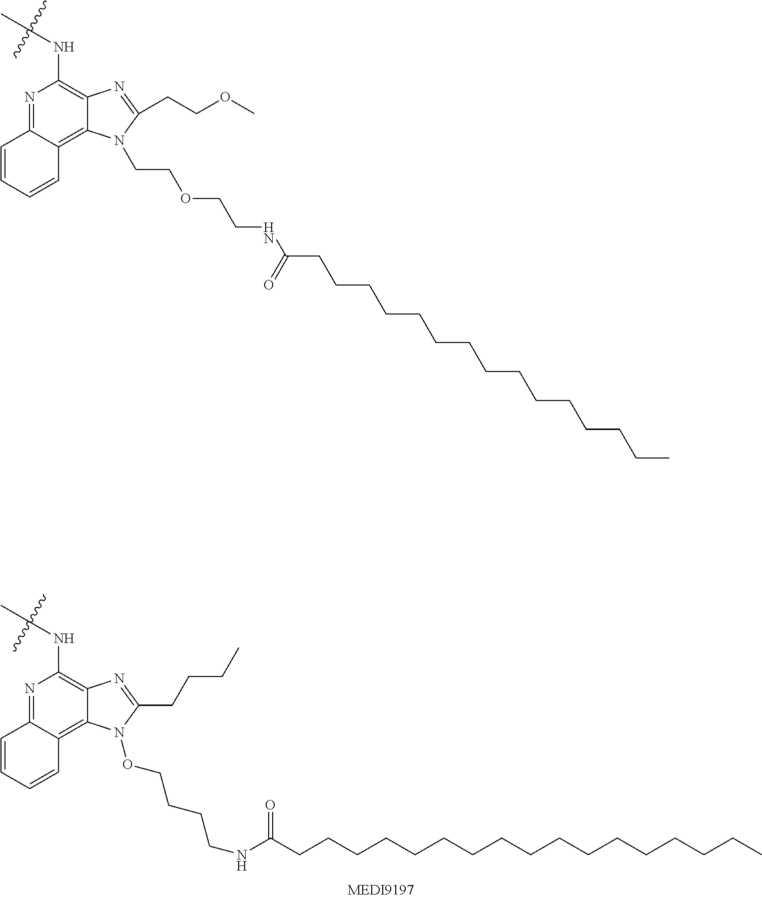 [SMILES: *Nc1nc2ccccc2c2c1nc(CCOC)n2CCOCCNC(=O)CCCCCCCCCCCCCCC ; *Nc1nc2ccccc2c2c1nc(CCCC)n2OCCCCNC(=O)CCCCCCCCCCCCCCCCC]

International Publication Nos. WO 2008/135791 and WO 2016/141092 also describe classes of imidazoquinoline compounds having immuno-modulating properties which act via TLR7.

Other examples of TRL agonists that be readily adapted for use as the drug moiety of the conjugates are disclosed in, for example, Yoo et al. “Structure-activity relationships in Toll-like receptor 7 agonistic 1H-imidazo[4,5-cjpyridines” Org. Biomol. Chem., 2013, 11, 6526-6545; Fletcher et al. “Masked oral prodrugs of Toll-like receptor 7 agonists: a new approach for the treatment of infectious disease”, 2006 Current opinion in investigational drugs (London, England). 7. 702-708; and Pryde et al. “The discovery of a novel prototype small molecule TLR7 agonist for the treatment of hepatitis C virus infection” Med. Chem. Commun., 2011, 2, 185-189.

Other non-limiting examples of TLR agonists for use in accordance with the present disclosure are described in International Publication No. WO 2019/236567, published Dec. 12, 2019, incorporated by reference herein.

It will also be appreciated by those skilled in the art that, particularly with the use of a self-immolative linker, the TRL agonists can be coupled to the linker though functional groups other than amines as shown above, such as through free hydroxyl groups for example.

Retinoic Acid-Inducible Gene I (RIG-I) Agonists

In some embodiments, the free drug moiety is a RIG-1 agonist. Retinoic acid-inducible gene I (RIG-1) agonists are used to induce innate immune responses. RIG-I is responsible for the type-1 interferon (IFN1) response. Upon its activation, RIG-I activates its RIG-I inflammasome, which leads to pyroptosis, an immunogenic mechanism of programmed cell death as well as the induction of pro-inflammatory cyotkines (Elion et al., Oncotarget. 2018; 9(48): 29007-29017). Pyroptosis results in the formation of pores in the plasma membrane, leading to hypotonic cell swelling and leakage of intracellular contents, including danger associated molecular patterns (DAMPs). The DAMPs and cytokines lead to a local acute inflammatory immune response.

Examples of RIG-I agonists include KIN700, KIN1148, KIN600, KIN500, KIN100, KIN101, KIN400, KIN2000, RGT100, and SD-9200.

Other non-limiting examples of RIG-I agonists for use in accordance with the present disclosure are described in International Publication No. WO 2019/236567, published Dec. 12, 2019, incorporated by reference herein.

iDASH Inhibitors

In some embodiments, the free drug moiety is an immuno-DASH inhibitor that inhibits the enzymatic activity of DPP8 and DPP9, and induces macrophage pyroptosis in vitro and/or in vivo. iDASH inhibitors are inhibitors of the of the DPP4, DPP8 and DPP9, post-proline cleaving enzymes, and act as checkpoint inhibitors of an immuno-checkpoint involving DASH enzymes. Inhibition of these target enzymes, which include both intracellular and extracellular targets, results in an increase in T cell cytotoxicity (release of cancer cell antigens, resulting in cancer cell death), an increase in dendritic cell trafficking (cancer antigen presentation), an increase in immunostimulatory cytokines (priming and activation of APCs and T cells), an increase in intratumoral CXCL10 half-life (trafficking of T cells to tumors), infiltration of T cells into tumors, recognition of cancer cells by T cells, and myeloid-derived suppressor cell (MDSC) depletion and impairment of MDSC immunosuppressive activity. Non-limiting examples of iDASH inhibitors are described in WO 2019/236567, the entire contents of which are incorporated herein by reference.

In some embodiments, the iDASH inhibitor is a boronic acid inhibitor of the DASH enzymes DPP8 and DPP9 (and in some embodiments also DPP-4).

In some embodiments, the iDASH inhibitor is a dipeptide boronic acid inhibitor of the DASH enzymes DPP8 and DPP9 (and in some embodiments also DPP-4). In some embodiments, the iDASH inhibitor the dipeptide boronic acid has a proline or proline analog in the P1 position. The subject iDASH inhibitors can mediate tumor regression by immune-mediated mechanisms. The subject iDASH inhibitors induce macrophage pyroptosis, and directly or indirectly have such activities as immunogenic modulation, sensitize tumor cells to antigen-specific CTL killing, alter immune-cell subsets and function, accelerate T cell priming via modulation of dendritic cell trafficking, and invoke a general T-cell mediated antitumor activity.

Examples of iDASH inhibitors include saxagliptin, sitagliptin, linagliptin, alogliptin, talabostat (Valine-boroProline; (PT100), vildagliptin, gemigliptin, anagliptin, teneligliptin, trelagliptin, omarigliptin, evogliptin, gosogliptin, dutogliptin.

In some embodiments, the immuno-DASH inhibitor for use in the method of the present disclosure are represented by the general formula;

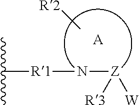

wherein
A represents a 4-8 membered heterocycle including the N and the Ca carbon;
Z represents C or N;
W represents —CN, CH═NR5,

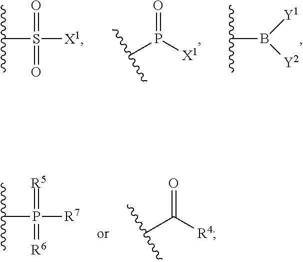

R′1 represents a C-terminally linked amino acid residue or amino acid analog, or a C-terminally linked peptide or peptide analog, the amine terminus of which forms a covalent with L1, or if L1 is a bond then with the substrate recognition sequence;
R′2 is absent or represents one or more substitutions to the ring A, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R7, —(CH₂)ₘ—OH, —(CH₂)ₘ—O-lower alkyl, —(CH₂)ₘ—O-lower alkenyl, —(CH₂)ₙ—O(CH₂)ₘ—R7, —(CH₂)ₘ—SH, —(CH₂)ₘ—S-lower alkyl, —(CH₂)ₘ—S-lower alkenyl, —(CH₂)ₙ—S—(CH₂)ₘ—R7;
if X is N, R′3 represents hydrogen, if X is C, R′3 represents hydrogen or a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R7, —(CH₂)ₘ—OH, —(CH₂)ₘ—O-lower alkyl, —(CH₂)ₘ—O-lower alkenyl, —(CH₂)ₙ—O(CH₂)ₘ—R7, —(CH₂)ₘ—SH, —(CH₂)ₘ—S-lower alkyl, —(CH₂)ₘ—S-lower alkenyl, —(CH₂)—S—(CH₂)ₘ—R7;
R5 represents H, an alkyl, an alkenyl, an alkynyl, —C(X1)(X2)X3, —(CH₂)ₘ—R7, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R7, —(CH₂)ₙ—SH, —(CH₂)ₙ—S-alkyl, —(CH₂)ₙ—S-alkenyl, —(CH₂)ₙ—S-alkynyl, —(CH₂)ₙ—S(CH₂)ₘ—R7, —C(O)C(O)ₙH2, —C(O)C(O)OR′7;
R6 represents hydrogen, a halogen, a alkyl, a alkenyl, a alkynyl, an aryl, —(CH₂)ₘ—R7, —(CH₂)ₘ—OH, —(CH₂)ₘ—O-lower alkyl, —(CH₂)ₘ—O-lower alkenyl, —(CH₂)ₙ—O(CH₂)ₘ—R7, —(CH₂)ₘ—SH, —(CH₂)ₘ—S-lower alkyl, —(CH₂)ₘ—S-lower alkenyl, —(CH₂)ₙ—S—(CH₂)ₘ—R7,
R7 represents, for each occurrence, a substituted or unsubstituted aryl, aralkyl, cycloalkyl, cycloalkenyl, or heterocycle;
R′7 represents, for each occurrence, hydrogen, or a substituted or unsubstituted alkyl, alkenyl, aryl, aralkyl, cycloalkyl, cycloalkenyl, or heterocycle; and
Y1 and Y2 can independently or together be OH, or a group capable of being hydrolyzed to a hydroxyl group, including cyclic derivatives where Y1 and Y2 are connected via a ring having from 5 to 8 atoms in the ring structure (such as pinacol or the like),
R50 represents O or S;
R51 represents N₃, SH₂, NH₂, NO₂ or O—R′7;
R52 represents hydrogen, a lower alkyl, an amine, OR′7, or a pharmaceutically acceptable salt, or R51 and R52 taken together with the phosphorous atom to which they are attached complete a heterocyclic ring having from 5 to 8 atoms in the ring structure
X1 represents a halogen;
X2 and X3 each represent a hydrogen or a halogen
m is zero or an integer in the range of 1 to 8; and
n is an integer in the range of 1 to 8.

In preferred embodiments, the ring A is a 5, 6 or 7 membered ring, e.g., represented by the formula

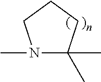

and more preferably a 5 or 6 membered ring (e.g., n is 1 or 2, though n may also be 3 or 4). The ring may, optionally, be further substituted.

In preferred embodiments, W represents

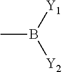

In preferred embodiments, R′1 is

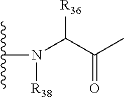

wherein R36 is a small hydrophobic group, e.g., a lower alkyl or a halogen and R38 is hydrogen, or R36 and R37 together form a 4-7 membered heterocycle including the N and the Cα carbon, as defined for A above.

In preferred embodiments, R′2 is absent, or represents a small hydrophobic group such as a lower alkyl or a halogen.

In preferred embodiments, R′3 is a hydrogen, or a small hydrophobic group such as a lower alkyl or a halogen.

In preferred embodiments, R′5 is a hydrogen, or a halogenated lower alkyl.

In preferred embodiments, X1 is a fluorine, and X2 and X3, if halogens, are fluorine.

Also deemed as equivalents are any compounds which can be hydrolytically converted into any of the aforementioned compounds including boronic acid esters and halides, and carbonyl equivalents including acetals, hemiacetals, ketals, and hemiketals, and cyclic dipeptide analogs.

In some preferred embodiments, the subject method utilizes, as a immuno-DASH inhibitor, a boronic acid analogs of an amino acid. For example, the present disclosure contemplates the use of boro-prolyl derivatives in the subject method. Exemplary boronic acid derived inhibitors of the present disclosure are represented by the general formula:

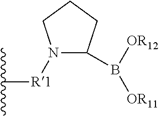

wherein
R′1 represents a C-terminally linked amino acid residue or amino acid analog, or a C-terminally linked peptide or peptide analog, the amine terminus of which forms a covalent with L1, or if L1 is a bond then with the substrate recognition sequence; and
R11 and R12 each independently represent hydrogen, a alkyl, or a pharmaceutically acceptable salt, or R11 and R12 taken together with the O—B—O atoms to which they are attached complete a heterocyclic ring having from 5 to 8 atoms in the ring structure.

In some embodiments, the immuno-DASH inhibitor is a peptide or peptidomimetic including a prolyl group or analog thereof in the P1 specificity position, and a nonpolar (and preferably hydrophobic) amino acid in the P2 specificity position, e.g., a nonpolar amino acid such as alanine, leucine, isoleucine, valine, proline, phenylalanine, tryptophan or methionine, or an analog thereof. In other embodiments, the P2 position an amino acid with charged sidechain, such as Arginine, Lysine, Aspartic acid or Glutamic Acid. For example, the immuno-DASH inhibitor may include an Ala-Pro or Val-Pro dipeptide sequence or equivalent thereof, and be represented in the general formulas:

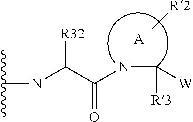

In preferred embodiments, the ring A is a 5, 6 or 7 membered ring, e.g., represented by the formula

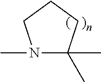

In some preferred embodiments, R32 is a small hydrophobic group, e.g., a lower alkyl or a halogen.

In some preferred embodiments, R32 is -lower alkyl-guanidine, -lower-alkyl-amine, lower-alkyl-C(O)OH, such as —(CH₂)ₘ—NH—C(═N)(NH₂), —(CH₂)ₘ—NH₂ or —(CH₂)ₘ—COOH, where m is 1-6, and preferably 1-3.

In preferred embodiments, R′2 is absent, or represents a small hydrophobic group such as a lower alkyl or a halogen.

In preferred embodiments, R′3 is a hydrogen, or a small hydrophobic group such as a lower alkyl or a halogen.

Another aspect of the disclosure relates to the immuno-DASH inhibitor represented by formula III, or a pharmaceutical salt thereof:

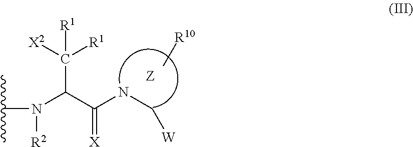

wherein
ring Z represents a 4-10 membered heterocycle including the N and the Ca carbon;
W represents —CN, —CH═NR4, a functional group which reacts with an active site residue of the target, or

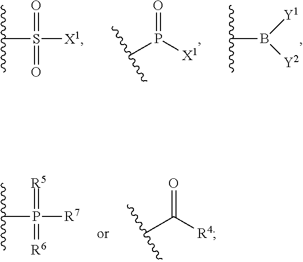

X is O or S;
X2 is H, a halogen, or a lower alkyl;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1 represents, independently for each occurrence, a halogen, a lower alkyl, a lower alkenyl, a lower alkynyl, a carbonyl, a thiocarbonyl, an amino, an acylamino, an amido, a cyano, a nitro, an azido, a sulfate, a sulfonate, a sulfonamido, —CF₃, —(CH₂)ₘ—R3, —(CH₂)ₘ—OH, —(CH₂)ₘ—O-lower alkyl, —(CH₂)ₘ—O-lower alkenyl, —(CH₂)ₙ—O(CH₂)ₘ—R3, —(CH₂)ₘ—SH, —(CH₂)ₘ—S-lower alkyl, —(CH₂)ₘ—S-lower alkenyl, or —(CH₂)ₙ—S—(CH₂)ₘ—R3;
R2 represents, for each occurrence, hydrogen, lower alkyl, lower alkynyl, —(CH₂)ₘ—R3, —C(═O)-alkyl, —C(═O)-alkenyl, —C(═O)-alkynyl, or —C(═O)—(CH₂)ₘ—R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH₂)ₘ—R3, —(CH₂)ₙ—OH, —(CH₂)ₙ—O-lower alkyl, —(CH₂)ₙ—O-alkenyl, —(CH₂)ₙ—O-alkynyl, —(CH₂)ₙ—O(CH₂)ₘ—R7, —(CH₂)ₙ—SH, —(CH₂)ₙ—S-lower alkyl, —(CH₂)ₙ—S-lower alkenyl, —(CH₂)ₙ—S-lower alkynyl, —(CH₂)ₙ—S—(CH₂)ₘ—R3, —C(O)C(O)NH₂, or —C(O)C(O)OR8;
R5 represents O or S;
R6 represents N3, SH, NH2, NO2 or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R10 is absent or represents one to three substitutions to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, lower alkyl-C(O)OH, —O-lower alkyl-C(O)OH, -guanidinyl; —(CH₂)ₘ—R7, —(CH₂)ₘ—OH, —(CH₂)ₘ—O-lower alkyl, —(CH₂)ₘ—O-lower alkenyl, —(CH₂)ₙ—O(CH₂)ₘ—R3, —(CH₂)ₘ—SH, —(CH₂)ₘ—S-lower alkyl, —(CH₂)ₘ—S-lower alkenyl, —(CH₂)ₘ—S—(CH₂)ₘ—R3;
n is 0, 1, 2, or 3; and
m is 0, 1, 2, or 3.

Another aspect of the disclosure relates to the immuno-DASH inhibitor represented by formula IV, or a pharmaceutical salt thereof:

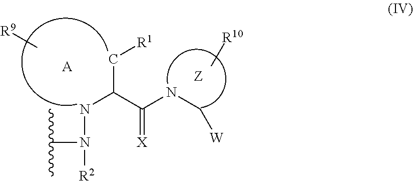

wherein
ring A represents a 3-10 membered ring structure including the N;
ring Z represents a 4-10 membered heterocycle including the N and the Ca carbon;
W represents —CN, —CH═NR4, a functional group which reacts with an active site residue of the target, or

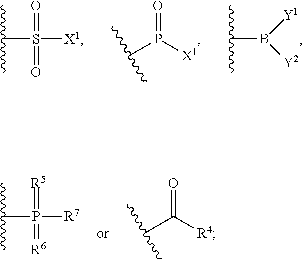

X is O or S;
X1 represents a halogen;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1 represents a halogen, a lower alkyl, a lower alkenyl, a lower alkynyl, a carbonyl, a thiocarbonyl, an amino, an acylamino, an amido, a cyano, a nitro, an azido, a sulfate, a sulfonate, a sulfonamido, —CF3, —(CH2)m-R3, —(CH2)mOH, —(CH2)m-O-lower alkyl, —(CH2)m-O-lower alkenyl, —(CH2)n-O—(CH2)m-R3, —(CH2)m-SH, —(CH2)m-S-lower alkyl, —(CH2)m-S-lower alkenyl, or —(CH2)n-S—(CH2)m-R3;
R2 represents, for each occurrence, hydrogen, lower alkyl, lower alkynyl, —(CH2)m-R3, —C(═O)-alkyl, —C(═O)-alkenyl, —C(═O)-alkynyl, or —C(═O)—(CH2)m-R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH2)m-R3, —(CH2)n-OH, —(CH2)n-O-lower alkyl, —(CH2)n-O-alkenyl, —(CH2)n-O-alkynyl, —(CH2)n-O—(CH2)m-R7, —(CH2)n-SH, —(CH2)n-S-lower alkyl, —(CH2)n-S-lower alkenyl, —(CH2)n-S-lower alkynyl, —(CH2)n-S—(CH2)m-R3, —C(O)C(O)NH2, or —C(O)C(O)OR8;
R5 represents O or S;
R6 represents N3, SH, NH2, NO2 or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R9 and R10, each independently, are absent or represents one to three substitutions to the ring A or to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, —(CH2)m-R7, —(CH2)m-OH, —(CH2)m-O-lower alkyl, —(CH2)m-O-lower alkenyl, —(CH2)n-O—(CH2)m-R3, —(CH2)m-SH, —(CH2)m-S-lower alkyl, —(CH2)m-S-lower alkenyl, —(CH2)n-S—(CH2)m-R3;
n is 0, 1, 2, or 3; and
m is 0, 1, 2, or 3.

In some preferred embodiments, the immuno-DASH inhibitor is a boronic acid inhibitor of the DASH enzymes DPP8 and DPP9 (and optionally also DPP-4 and/or FAP).

In some preferred embodiments, the immuno-DASH inhibitor is a dipeptide boronic acid inhibitor of the DASH enzymes DPP8 and DPP9 (and optionally also DPP-4 and/or FAP). In some preferred embodiments, the immuno-DASH inhibitor the dipeptide boronic acid has a proline or proline analog in the P1 position. The subject immuno-DASH inhibitors can mediate tumor regression by immune-mediated mechanisms. The subject immuno-DASH inhibitors induce macrophage pyroptosis, and directly or indirectly have such activities as immunogenic modulation, sensitize tumor cells to antigen-specific CTL killing, alter immune-cell subsets and function, accelerate T cell priming via modulation of dendritic cell trafficking, and invoke a general T-cell mediated antitumor activity.

In some embodiments, the subject combination of immuno-DASH inhibitor and PD-1 inhibitor can be administered as part of a therapy involving one or more other chemotherapeutic agents, immuno-oncology agents or radiation. It can also be used a part of therapy including tumor vaccines, adoptive cell therapy, gene therapy, oncolytic viral therapies and the like.

In some embodiments, the immuno-DASH inhibitor of the present methods is represented by formula I, or a pharmaceutical salt thereof:

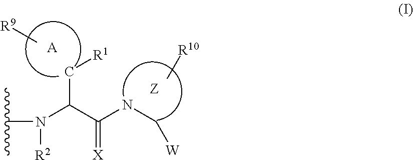

wherein
ring A represents a 3-10 membered ring structure;
ring Z represents a 4-10 membered heterocycle including the N and the Ca carbon;
W represents —CN, CH═NR4, a functional group which reacts with an active site residue of the target, or

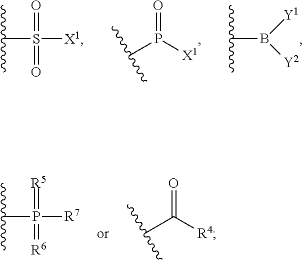

X is O or S;
X1 represents a halogen;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1 represents a halogen, a lower alkyl, a lower alkenyl, a lower alkynyl, a carbonyl, a thiocarbonyl, an amino, an acylamino, an amido, a cyano, a nitro, an azido, a sulfate, a sulfonate, a sulfonamido, —CF3, —(CH2)m-R3, —(CH2)mOH, —(CH2)m-O-lower alkyl, —(CH2)m-O-lower alkenyl, —(CH2)n-O—(CH2)m-R3, —(CH2)m-SH, —(CH2)m-S-lower alkyl, —(CH2)m-S-lower alkenyl, or —(CH2)n-S—(CH2)m-R3;
R2 represents, for each occurrence, hydrogen, lower alkyl, lower alkynyl, —(CH2)m-R3, —C(═O)-alkyl, —C(═O)-alkenyl, —C(═O)-alkynyl, or —C(═O)—(CH2)m-R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH2)m-R3, —(CH2)n-OH, —(CH2)n-O-lower alkyl, —(CH2)n-O-alkenyl, —(CH2)n-O-alkynyl, —(CH2)n-O—(CH2)m-R7, —(CH2)n-SH, —(CH2)n-S-lower alkyl, —(CH2)n-S-lower alkenyl, —(CH2)n-S-lower alkynyl, —(CH2)n-S—(CH2)m-R3, —C(O)C(O)NH2, or —C(O)C(O)OR8;
R5 represents O or S;
R6 represents N3, SH, NH2, NO2 or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R9 and R10, each independently, are absent or represents one, two, or three substitutions to the ring A or to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, lower alkyl-C(O)OH, —O-lower alkyl-C(O)OH, -guanidinyl; —(CH2)m-R7, —(CH2)m-OH, —(CH2)m-O-lower alkyl, —(CH2)m-O-lower alkenyl, —(CH2)n-O—(CH2)m-R3, —(CH2)m-SH, —(CH2)m-S-lower alkyl, —(CH2)m-S-lower alkenyl, —(CH2)n-S—(CH2)m-R3;
n is 0, 1, 2, or 3; and
m is 0, 1, 2, or 3.

In some embodiments, the immuno-DASH inhibitor of Formula I is represented in Formula Ia, or is a pharmaceutical salt thereof:

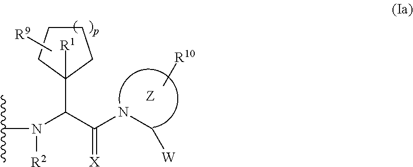

wherein X, W, Z, R1, R2, R9 and R10 are as defined above for Formula I, and p is 1, 2 or 3.

In some preferred embodiments of Ia: R1 is a lower alkyl; R9 is absent, or independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor of Formula I is represented in Formula Ib, or is a pharmaceutical salt thereof:

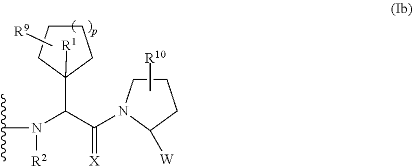

wherein X, W, R1, R2, R9 and R10 are as defined above for Formula I, and p is 1, 2 or 3.

In some preferred embodiments of Ib: R1 is a lower alkyl; R9 is absent, or independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor of Formula I is represented in Formula Ic, or is a pharmaceutical salt thereof:

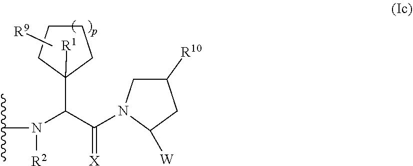

wherein X, W, R1, R2, R9 and R10 are as defined above for Formula I, and p is 1, 2 or 3.

In some preferred embodiments of Ic: R1 is a lower alkyl; R9 is absent, or independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor is represented by:

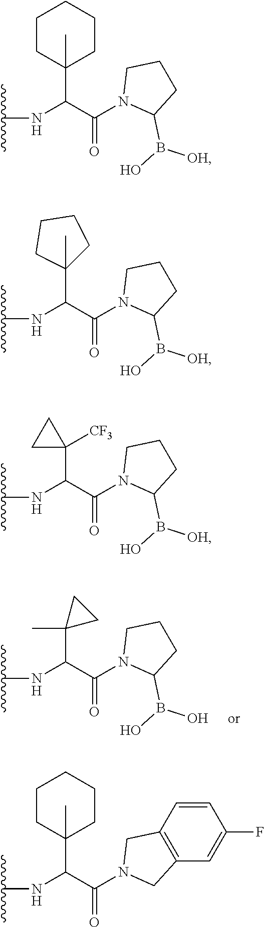

Another aspect of the disclosure relates to the immuno-DASH inhibitor represented by formula II, or a pharmaceutical salt thereof:

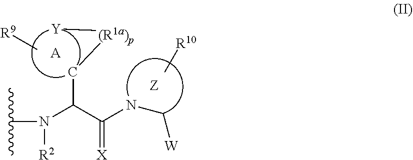

wherein
ring A, along with each occurrence of R1a, represents a 7-12 membered polycyclic ring structure;
ring Z represents a 4-10 membered heterocycle including the N and the Ca carbon;
W represents —CN, CH═NR4, a functional group which reacts with an active site residue of the target, or

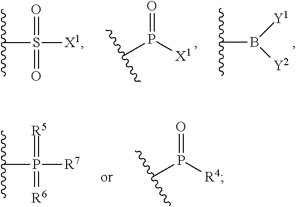

X is O or S;
X1 represents a halogen;
Y is C or N;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1a represents a lower alkyl, —(CH2)m-, —(CH2)m-O—(CH2)m-; —(CH2)m-N—(CH2)m-; or —(CH2)m-S—(CH2)m;
R2 represents, for each occurrence, hydrogen, lower alkyl, lower alkynyl, —(CH2)m
R3, C(═O)-alkyl, C(═O)-alkenyl, C(═O)-alkynyl, or C(═O)—(CH2)m-R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH2)m
R3, —(CH2)n-OH, —(CH2)n-O-lower alkyl, —(CH2)n-O-alkenyl, —(CH2)n-O-alkynyl, —(CH2)n-O—(CH2)m-R7, —(CH2)n-SH, —(CH2)n-S-lower alkyl, —(CH2)n-S-lower alkenyl, —(CH2)n-S-lower alkynyl, —(CH2)n-S—(CH2)m-R3, —C(O)C(O)NH2, or —C(O)C(O)OR8;
R5 represents O or S;
R6 represents N3, SH, NH2, NO2 or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R9 and R10, each independently, are absent or represents one, two, or three substitutions to the ring A or to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, lower alkyl-C(O)OH, —O-lower alkyl-C(O)OH, -guanidinyl; —(CH2)m-R7, —(CH2)m-OH, —(CH2)m-O-lower alkyl, —(CH2)m-O-lower alkenyl, —(CH2)n-O—(CH2)m-R3, —(CH2)m-SH, —(CH2)m-S-lower alkyl, —(CH2)m-S-lower alkenyl, —(CH2)n-S—(CH2)m-R3;
n is 0, 1, 2, or 3;
m is 0, 1, 2, or 3; and
p is 1, 2, or 3.

In some embodiments, the immuno-DASH inhibitor of Formula II is represented in Formula IIa, or is a pharmaceutical salt thereof:

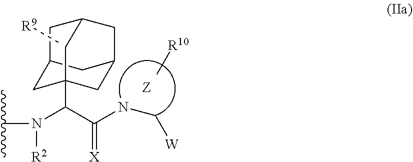

wherein X, W, Z, R2, R9 and R10 are as defined above for Formula II.

In some preferred embodiments of IIa: R9, independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor of Formula II is represented in Formula IIb, or is a pharmaceutical salt thereof:

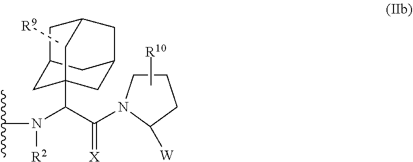

wherein X, W, R2, R9 and R10 are as defined above for Formula II.

In some preferred embodiments of IIb: R9, independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor of Formula II is represented in Formula IIc, or is a pharmaceutical salt thereof:

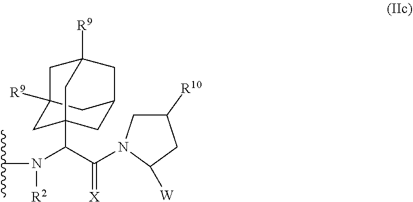

wherein X, W, R2, R9 and R10 are as defined above for Formula II.

In some preferred embodiments of IIe: R9, independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor of Formula II is represented in Formula IId, or is a pharmaceutical salt thereof:

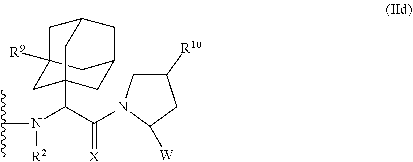

wherein X, W, R2, R9 and R10 are as defined above for Formula II.

In some preferred embodiments of IId: R9, independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; and W is —B(OH)2 or —CN (and more preferably —B(OH)2).

In some embodiments, the immuno-DASH inhibitor of Formula II is represented in Formula IIe, or is a pharmaceutical salt thereof:

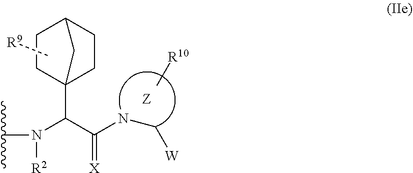

wherein X, W, Z, R2, R9 and R10 are as defined above for Formula II.

In some preferred embodiments of IIe: R9, independently for each occurrence, is a lower alkyl, —OH, —NH2, —N3, -lower alkyl-C(O)OH, —O-lower alkyl, —O-lower alkyl-C(O)OH, -guanidinyl; X is O; each R2 is hydrogen, R10 is absent, or represents a single substitution of —OH, —NH2, —CN or —N3; Z is a pyrrolidine or piperidine ring (and more preferably a pyrrolidine ring); and W is —B(OH)2 or —CN (and more preferably —B(OH)2).

In some embodiments, the immuno-DASH inhibitor is one of the following:

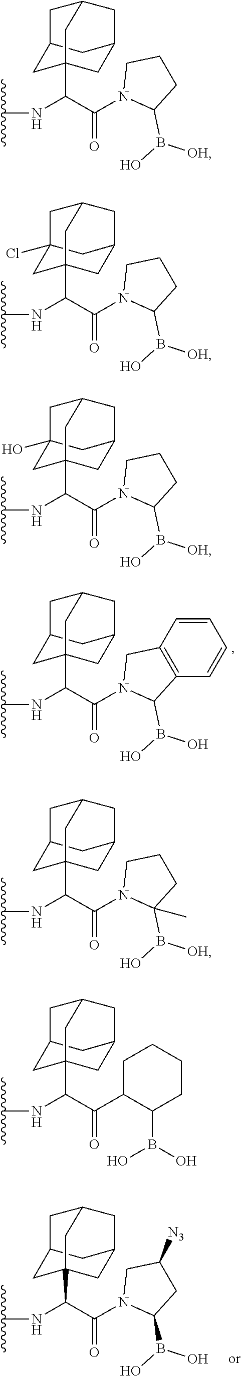

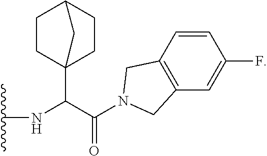 [SMILES: *NC(C(=O)N1Cc2ccc(F)cc2C1)C12CCC(CC1)C2]

Other non-limiting examples of iDASH inhibitors for use in accordance with the present disclosure are described in International Publication No. WO 2019/236567, published Dec. 12, 2019, incorporated by reference herein.

Stimulator of Interferon Genes Protein (STING) Agonists

In some embodiments, the free drug moiety is a STING agonist. Stimulator of interferon genes protein (STING) agonists bind to STING, activating the STING pathway, which promotes IκK-related kinase TANK-binding kinase 1 (TBK1) signaling. TBK1 signaling activates nuclear factor-kappa B (NF-kB) and interferon regulatory factor 3 (IRF3) in immune cells in the tumor microenvironment. Their activation leads to the production of pro-inflammatory cytokines, including interferons (IFNs). Expression of IFN-beta promotes the cross-presentation of tumor-associated antigens by CD8α+ and CD103+ dendritic cells to cytotoxic T lymphocytes (CTLs). This results in a CTL-mediated immune response against tumor cells and causes tumor cell lysis.

STING agonists include cyclic dinucleotides and derivatives thereof, such as modified cyclic dinucleotides including, for example, modified nucleobases, modified ribose or modified phosphate linkages. In some embodiments, the modified cyclic dinucleotide comprises a modified phosphate linkage, e.g., thiophosphate. In some embodiment, the STING agonist comprises a cyclic dinucleotide (e.g., a modified cyclic dinucleotide) having 2′,5′ or 3′,5′ phosphate linkages. In some embodiments, STING agonists include cyclic dinucleotides that have Rp or Sp stereochemistry around the phosphate bond.

In some embodiments, the STING agonist is Rp, Rp dithio 2′,3′ c-di-AMP (eg, Rp, Rp-dithio c-[A (2′,5′) pA (3′,5′) p]) or its cyclic dinucleotide analog. In some embodiments, the STING agonist is a compound described in US Patent Publication US 2015/0056224. In some embodiments, the STING agonist is c-[G (2′,5′) pG (3′,5′) p], a dithioribose O-substituted derivative thereof. In some embodiments, the STING agonist is c-[A (2′,5′) pA (3′,5′) p] or a dithioribose O-substituted derivative thereof. In some embodiments, the STING agonist is c-[G (2′,5′) pA (3′,5′) p] or a dithioribose O-substituted derivative thereof. In some embodiments, the STING agonist is 2′-O-propargyl-cyclic-[A (2′,5′) pA (3′,5′) p] (2′-O-propargyl-ML-CDA).

STING agonists also include xanthenone and derivatives thereof, including flavone acetic acid (FAA), xanthene-acetic acid (XAA), dimethylxanthenone-4-acetic acid (DMXAA), and derivatives thereof. Examples of STING agonists include ADU-S100 (ML-RR-S2-CDA or MIW815), MK-1454, vadimezan (5,6-dimethylxanthenone-4-acetic acid (DMXAA)), 2′3′-cGAMP, c-di-GMP, 3′3′-cGAMP, and ML-RR-CDA.

Non-limiting examples of STING agonists include agonists represented in the one of the general formulas

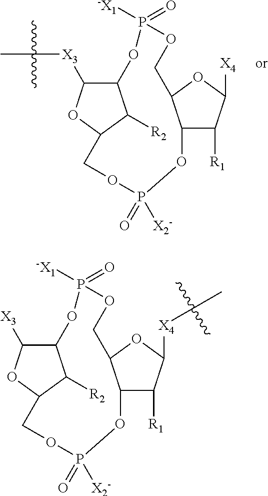

wherein
X₁ and X₂ are, independently, O or S, and preferably are the same (O,O or S,S);
X₃ and X₄ are, independently, a purine, such as a guanine or guanine analog, or a pymridine, and wherein the wavy lines indicate covalent attachment site to L1, or where L1 is a bond, to the substrate recognition sequence;
R₁ and R₂ are, independently, H, hydroxyl, a halogen (preferably F or Cl) or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, —OH, ═O, —COOR′ or —OR′, wherein R₁ and R₂ are not both H,
R′ is H or lower alkyl, —CH₂OH, or —CONH₂.

In some embodiments, the STING agonist is represented in one of the formula:

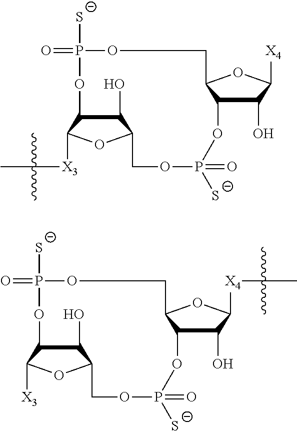

In the STING agonist structures above, X₃ and X₄ may each independently be, for example, 9-purine, 9-adenine, 9-guanine, 9-hypoxanthine, 9-xanthine, 9-uric acid, or 9-isoguanine, provided that one of X3 or X4 includes a functional group with which L² shares a bond if L² is a self immolative linker, or a functional group with which DM shares a bond if L² is (that) a bond.

X₃ and X₄ may be identical or different.

In some embodiments, the STING agonists may be provided in the form of predominantly Rp,Rp or Rp,Sp stereoisomers. In some embodiments, the STING agonists may be provided in the form of predominantly Rp,Rp stereoisomers.

Exemplary STING agonists include:

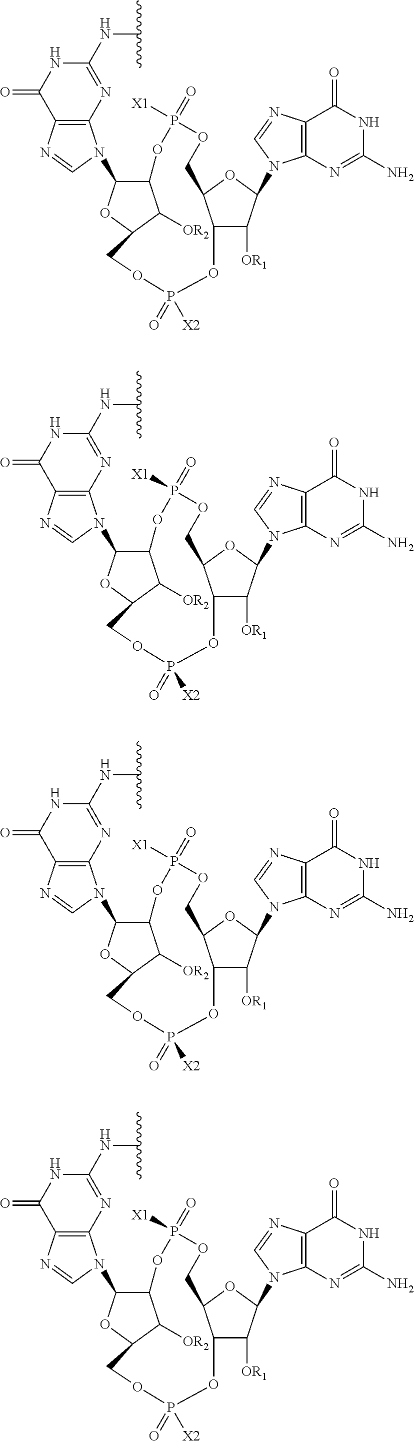

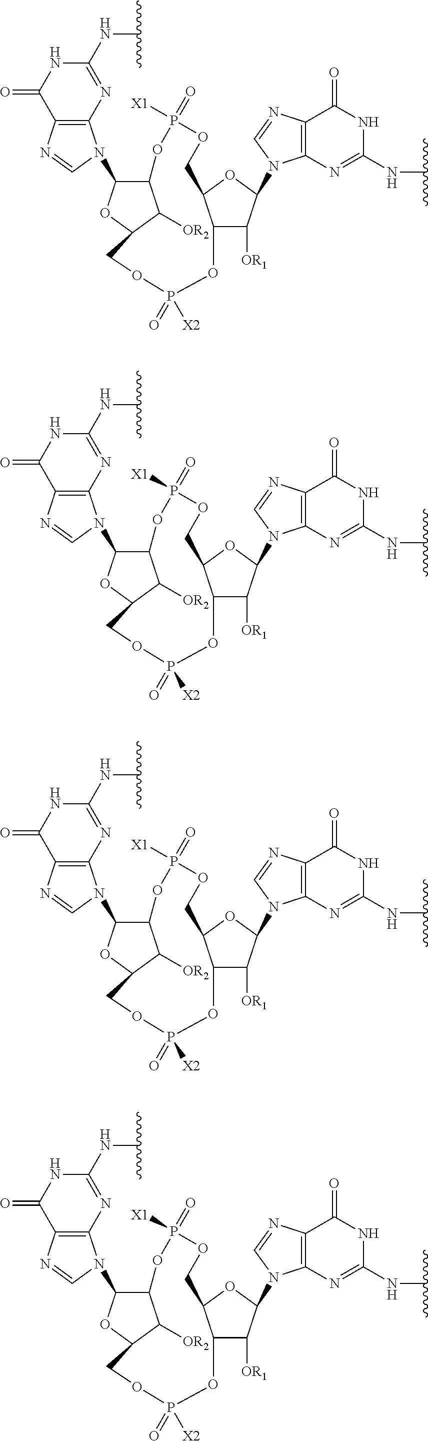

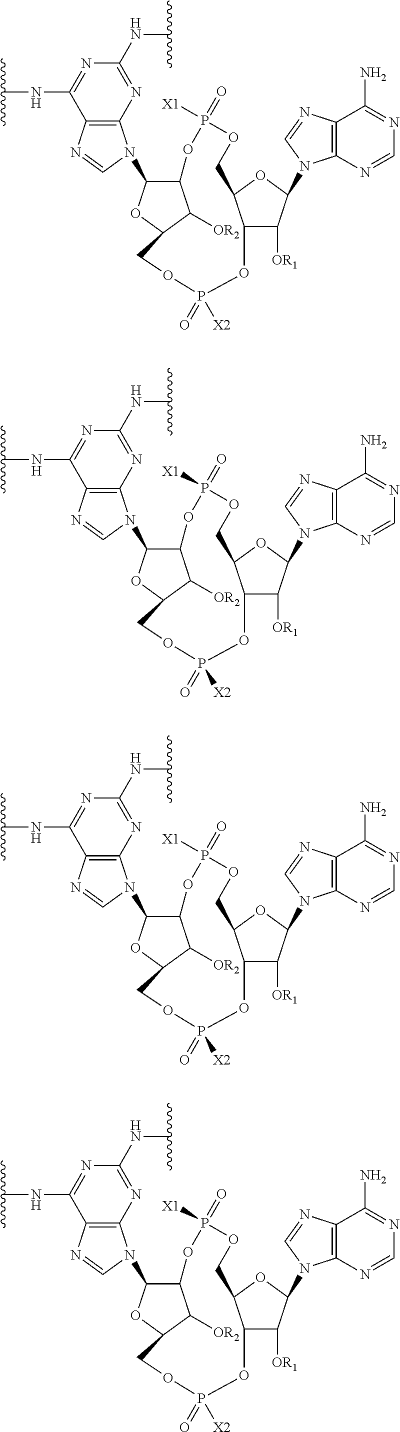

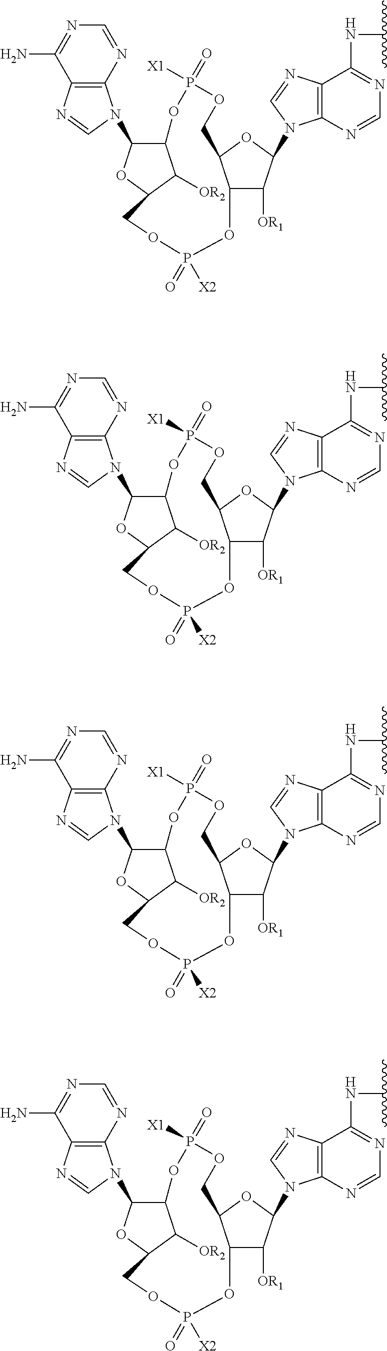

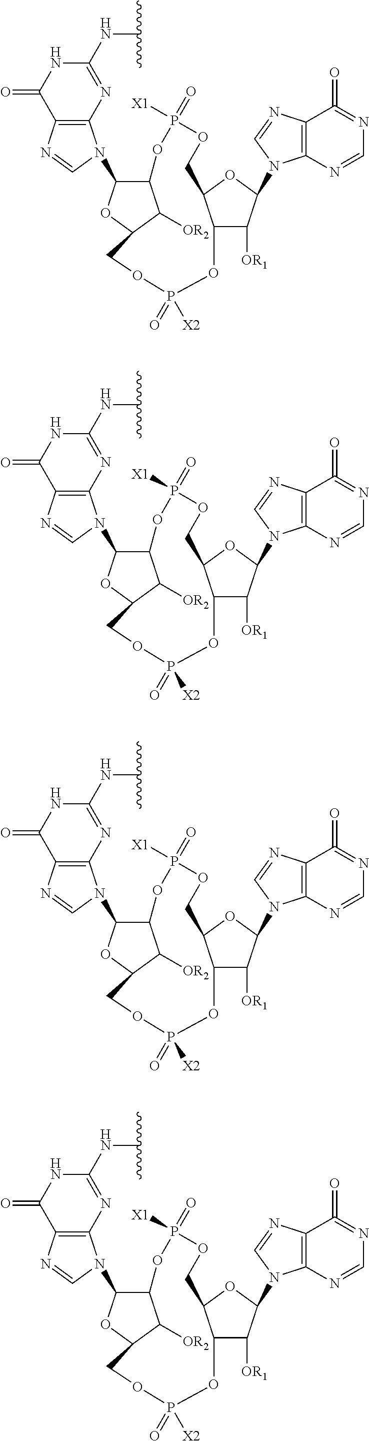

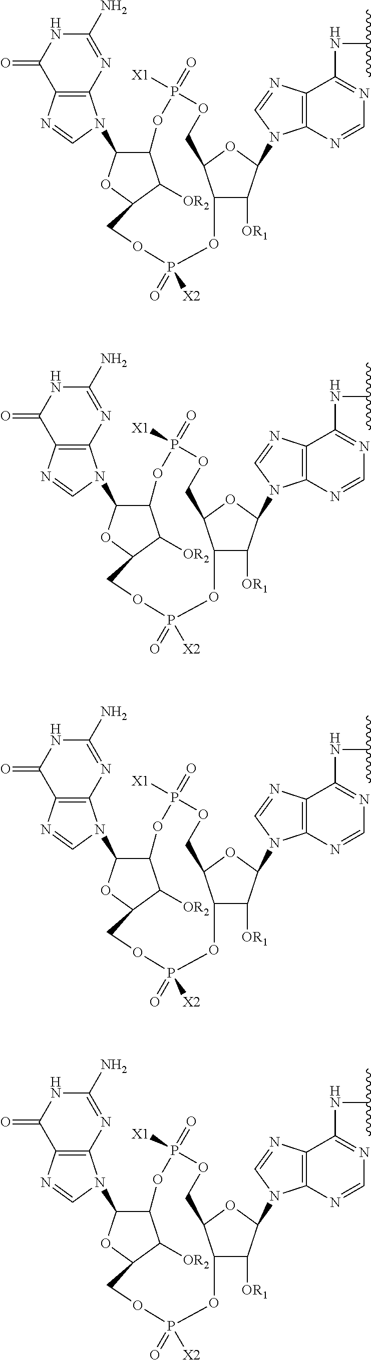

In some embodiments, the STING agonist is represented in one of the following structures”

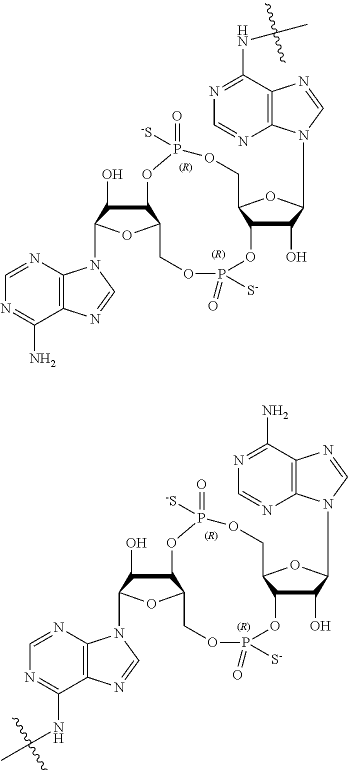

Still another STING agonist that can be used as Drug Moiety in the present binder conjugates is

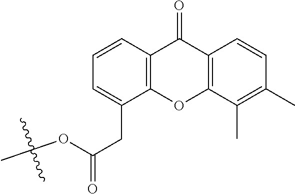 [SMILES: *OC(=O)Cc1cccc2c(=O)c3ccc(C)c(C)c3oc12]

It will also be appreciated by those skilled in the art that, particularly with the use of a self-immolative linker, the STING agonist can be coupled to the linker though functional groups other than amines as shown above, such as through free hydroxyl groups for example.

Non-limiting examples of STING agonists for use in accordance with the present disclosure are described in International Publication No. WO 2019/236567, published Dec. 12, 2019, incorporated by reference herein. Additional examples of STING agonists are described in International Publication Nos. WO 2017/123669 and WO 2015/077354 as well as US Patent Publication No. US 2015/0056224, each of which is hereby incorporated by reference.

It will also be appreciated by those skilled in the art that, particularly with the use of a self-immolative linker, the STING agonist can be coupled to the linker though functional groups other than amines as shown above, such as through free hydroxyl groups for example.

Radiopharmaceuticals

In some embodiments, the drug moiety is a radiopharmaceutical. For example, the drug moiety can include a chelator for a radionuclide useful for radiotherapy or imaging procedures. Radionuclides useful within the present disclosure include gamma-emitters, positron-emitters, Auger electron-emitters, X-ray emitters and fluorescence-emitters, with beta- or alpha-emitters for therapeutic use. Examples of radionuclides useful as toxins in radiation therapy include: ⁴³K, ⁴⁷Sc, ⁵¹Cr, ⁵⁷Co, ⁵⁸Co, ⁵⁹Fe, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁸¹Rb, ⁹⁰Y, ⁹⁷Ru, ^{99m}Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, 128Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Conditions under which a chelator will coordinate a metal are described, for example, by Gansow et al., U.S. Pat. Nos. 4,831,175, 4,454,106 and 4,472,509. Examples of chelators includes, 1,4,7-triazacyclononane-N,N′,N″-triacetic acid (NOTA) 1,4,7,10-tetraazacyclododecane-N,N′,N″,N′″-tetraacetic acid (DOTA) 1,4,8,11-tetraazacyclotetradecane-N,N′,N″,N′″-tetraacetic acid (TETA).

### Therapeutic Conjugate Compositions and Methods of Use

In some embodiments, a therapeutic conjugate is formulated with a pharmaceutically acceptable excipient to form a composition. In some embodiments, an enzyme-cleavable linker or an SRS is formulated with a pharmaceutically acceptable excipient to form a composition. A molecule or other substance/agent is considered “pharmaceutically acceptable” if it is approved or approvable by a regulatory agency of the Federal government or a state government or listed in the U.S. Pharmacopeia or other generally recognized pharmacopeia for use in animals, including humans. An excipient may be any inert (inactive), non-toxic agent, administered in combination with a therapeutic conjugate. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

In some embodiments, a therapeutic conjugate or composition is administered to a subject. A subject may be any animal (e.g., a mammal), including, but not limited to, humans, non-human primates, canines, felines, and rodents. A “subject” refers to a human subject. In some embodiments, the subject has a diseased tissue, such as a cancer.

Therefore, a therapeutic conjugate or composition may be administered to a subject to treat a diseased tissue, such as cancer. In some embodiments, a therapeutic conjugate is used to manufacture a medicament for the treatment of a diseased tissue (e.g., cancer). Non-limiting examples of cancers include skin cancer (e.g., melanoma or non-melanoma, such as basal cell or squamous cell), lung cancer, prostate cancer, breast cancer, colorectal cancer, kidney (renal) cancer, bladder cancer, non-Hodgkin's lymphoma, thyroid cancer, endometrial cancer, exocrine cancer, and pancreatic cancer. Other cancers are contemplated herein. The term treat, as known in the art, refers to the process of alleviating at least one symptom associated with a disease (e.g., cancer). A symptom may be a physical, mental, or pathological manifestation of a disease. Symptoms associated with various diseases are known. To treat or prevent a particular condition, a therapeutic conjugate as provided herein should be administered in an effective amount, which can be any amount used to treat or prevent the condition. Thus, in some embodiments, an effective amount is an amount used to alleviate a symptom associated with the particular disease being treated. Methods are known for determining effective amounts of various therapeutic molecules, for example.

Routes of administration include intravenous, intramuscular, intratumoral, intraperitoneal, intranasal, and subcutaneous. Other routes of administration are encompassed by the present disclosure.

Thus, the therapeutic conjugates of the present disclosure may be formulated for intravenous, intramuscular, intratumoral, intraperitoneal, intranasal, or subcutaneous administration.

### EXAMPLES

### Example 1—Anti-Tumor Activity of Therapeutic Conjugates

Therapeutic conjugates comprising Fc conjugated to talabostat (Val-boroPro) via an enzyme-cleavable linker were generated and injected into a syngeneic model, CT26-mFAP+ mice, which are used for mouse colon cancer and have a FAPα gene knock-in. The tumor volume in the mice was measured nine times over 20 days. As can be seen in FIG. 2, the mice injected with the therapeutic conjugates (“SQT-Gly” groups) had lower tumor volumes than some of their counterpart mice injected with therapeutic conjugates comprising a cell-binding moiety (the “AVA04-182” groups) and the controls. Without wishing to be bound by theory, it is thought that the AVA04-182 groups did not work as well as the SQT-Glyp groups because they were cleared by anti-drug antibodies.

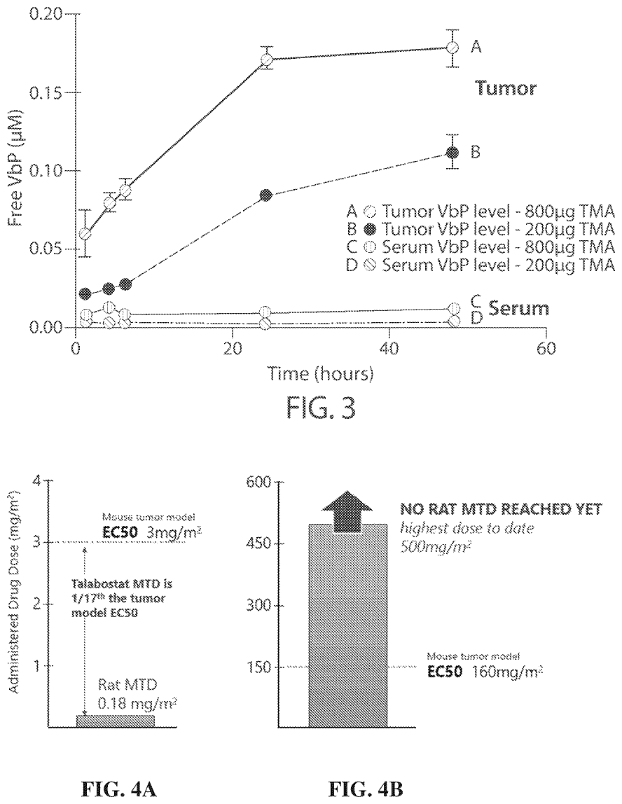

A pharmacokinetic study was also undertaken. The therapeutic conjugates were also injected in different dose and the amount of free talabostat was measured over 48 hours. As can be seen in FIG. 3, the therapeutic conjugates show preferential intratumoral exposure to the FAP-released iDASH inhibitor.

### Example 2—EC₅₀ Studies

Half-maximal effective concentration (EC₅₀) studies were undertaken to examine the difference between mice and rats administered talabostat alone (FIG. 4A) and mice and rats administered the therapeutic conjugates comprising talabostat (FIG. 4B). When talabostat is administered alone, the talabostat maximum tolerated dose (MTD) is about 3 mg/m² in a mouse tumor model and 0.18 mg/m² in rats. That is, the talabostat MTD is 1/17 the tumor model EC₅₀ (FIG. 4A). In contrast, when the talabostat is administered as part of the therapeutic conjugate, the MTD is 160 mg/m² in the mouse model and the EC₅₀ for rats is greater than 500 mg/m² (FIG. 4B). Talabostat, as part of the therapeutic conjugate, can be administered to rats and mice at therapeutically effective doses.

### Example 3—In Vivo Tumor Studies

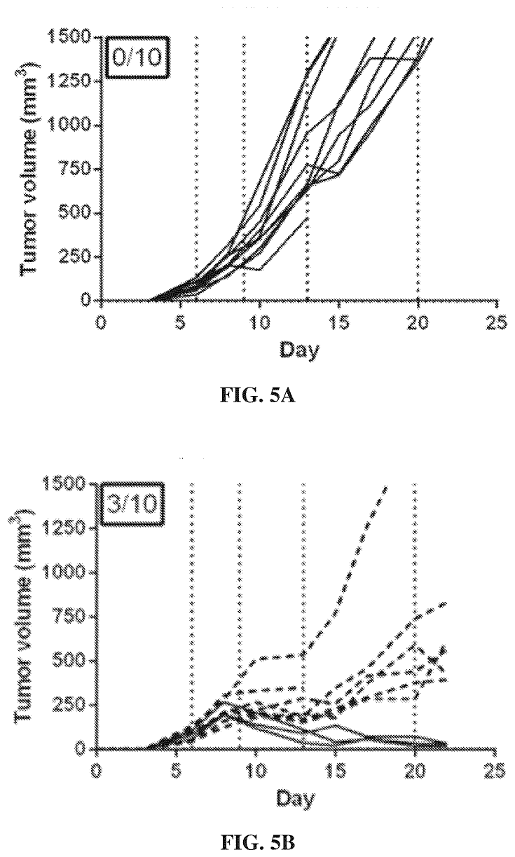

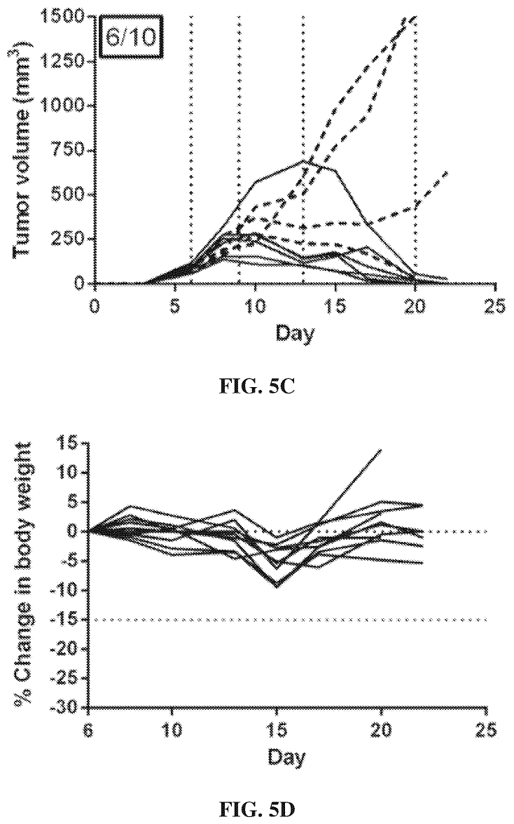

CT26-mFAP+mice were injected with vehicle (FIG. 5A), a therapeutic conjugate comprising a cell-binding moiety (FIG. 5B), or Fc conjugated to talabostat (Val-boroPro) via a FAPα-cleavable linker (FIG. 5C), and tumor volume was measured over 22 days. In the vehicle group, zero of the 10 mice showed a reduction in tumor volume and all mice were sacrificed before the end of the study (around day 20). In the group that received the therapeutic conjugate comprising a cell-binding moiety, three of the 10 mice had tumor volumes that decreased back to baseline. In contrast, the therapeutic conjugates described herein (without a cell binding moiety) resulted in six out of ten mice having tumor volumes that decreased back to baseline during the study. Further, the body weights of the mice were measured during the study and the percent change in body weight was calculated (FIG. 5D). None of the mice measured had a percent change in body weight of greater than 15%.

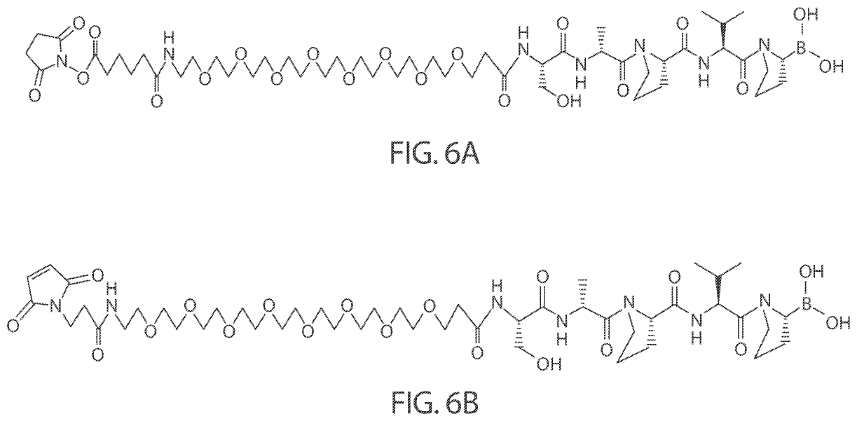

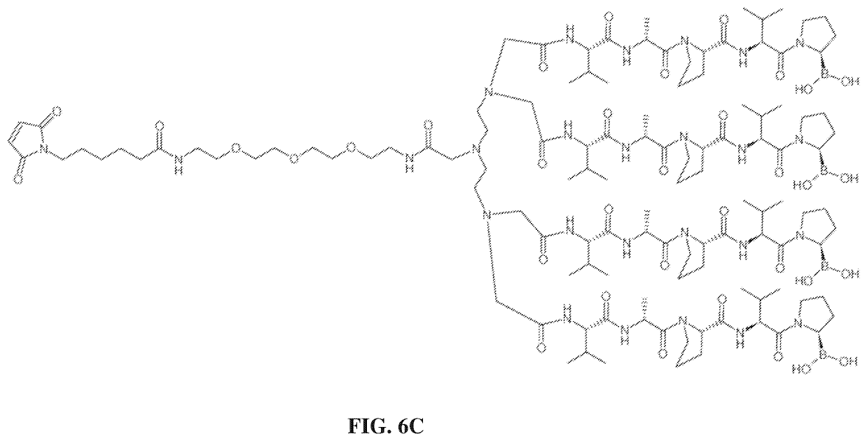

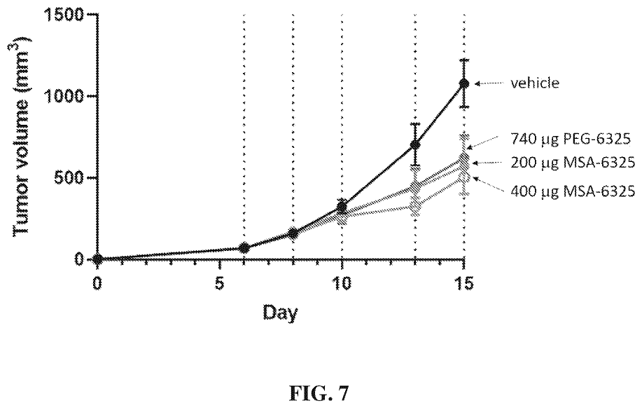

In another set of experiments, CT26-mFAP+mice were injected with vehicle (control), 200 μg or 400 μg MSA-6325 (mouse serum albumin conjugated with 6325), or 740 μg PEG-6325 (6325 reacted with SUNBRIGHT PTE-200 PA, a 20 KDa 4 arm functional PEG), and change in tumor volume was measured over time (FIG. 7). “6325” (FIG. 6A) is an exemplary FAP-activated I-DASH inhibitor including an NHS group for conjugation to lysine residues of proteins.

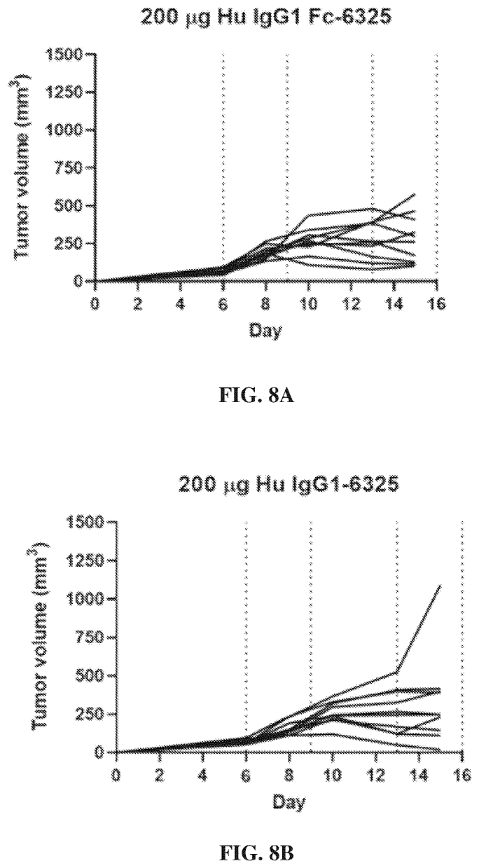

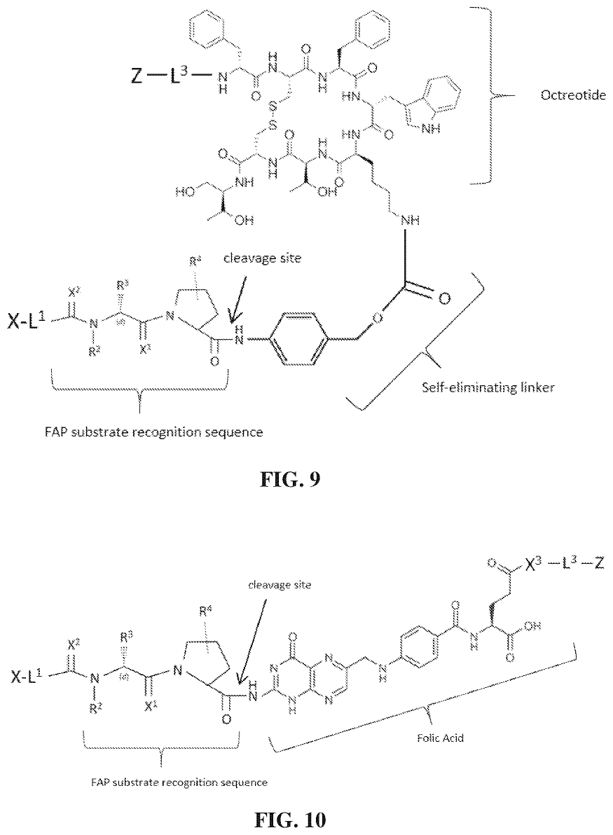

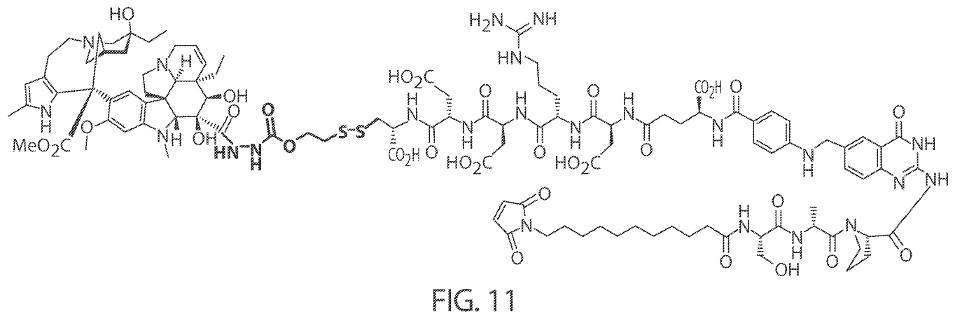

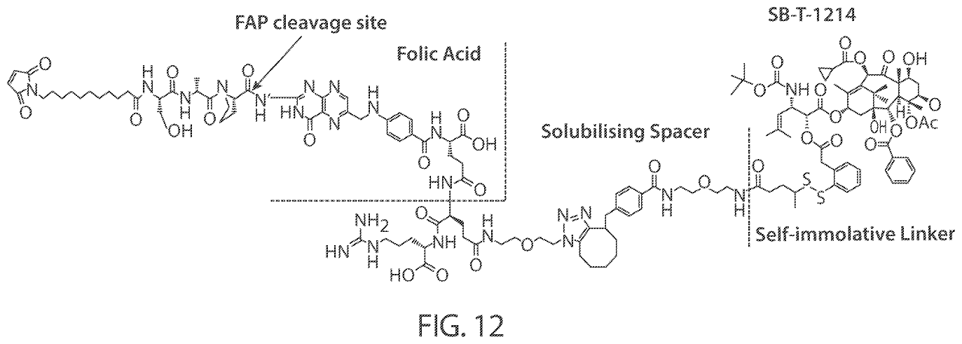

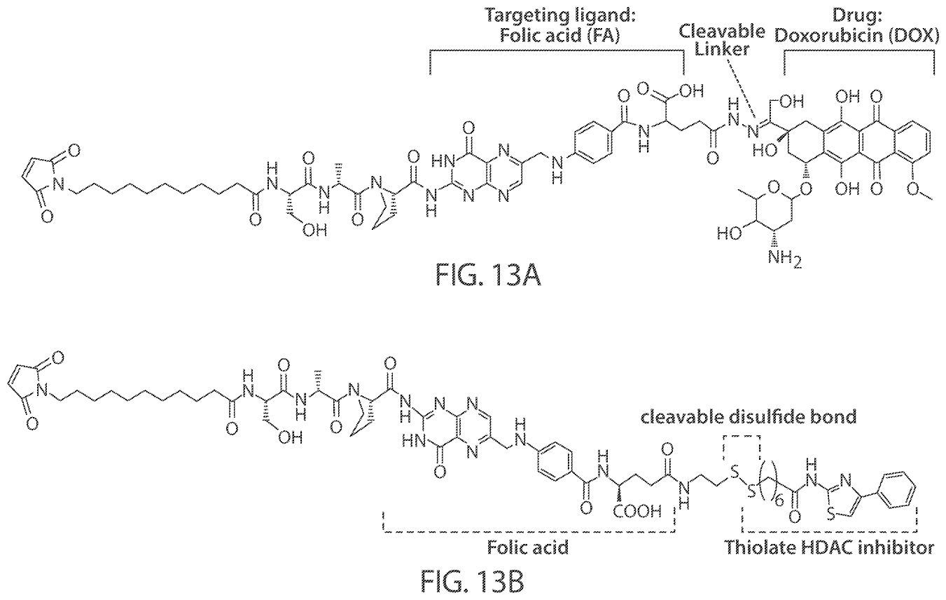

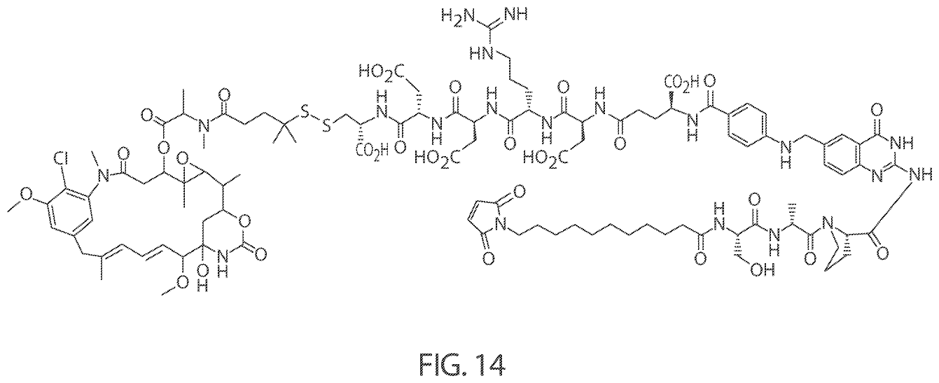

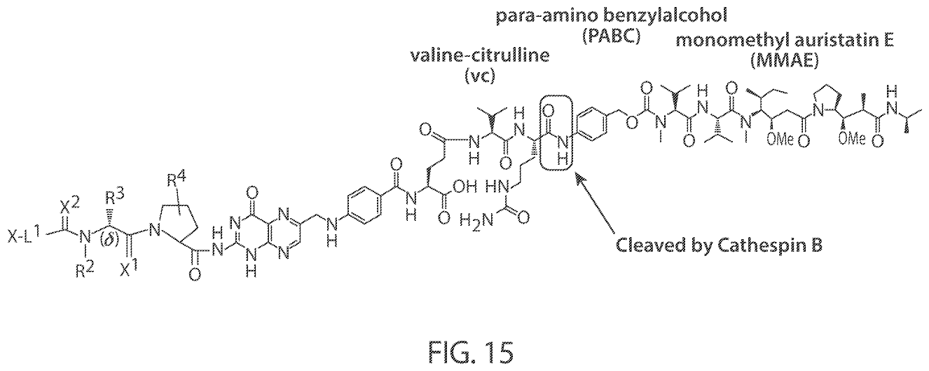

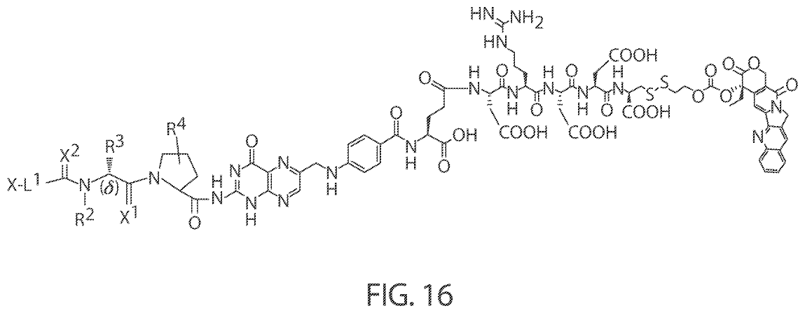

In yet another set of experiments, CT26-mFAP+mice were injected with either a human Fc fragment conjugated with 6325 (FIG. 8A), or a full human IgG antibody conjugated with 6325 (FIG. 8B) and change in tumor volume was measured over time.

### Example 4—In Vitro Studies

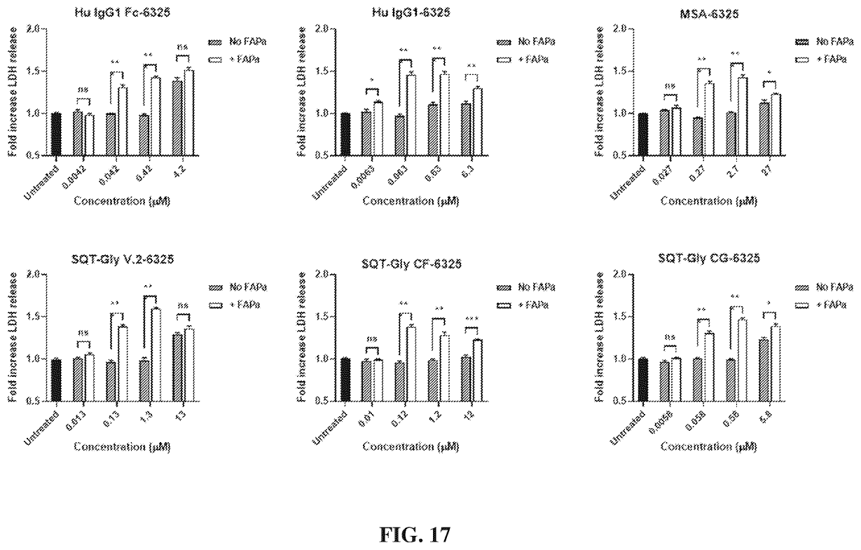

In further in vitro studies, several conjugates were tested for their ability to induce pyroptosis in J774 cells in the absence and presence of FAPα. Briefly, J774A.1 mouse macrophage cell line cells were cultured in triplicate wells with serial 10-fold dilutions of either VbP (data not shown) or one of the FAP-activated I-DASH inhibitors (Hu IgG1 Fc-6325, Hu IgG1-6325, MSA-6325, SQT-Gly V.2-6325, SQT-Gly CF-6325 or SQT-Gly CG-6325) in the presence or absence of rhFAPα at a final concentration of 25 nM for 24 hours. Each experiment included an untreated control. Following the incubation, the levels of lactate dehydrogenase (LDH, a marker of pyroptosis) released into culture supernatants were measured using the CytoTox 96 Non-radioactive Cytotoxicity Assay according to the manufacturer's instructions. The results are shown in FIG. 17 as fold-increase in LDH release. All conjugates tested exhibited a clear differentiation with or without FAPα that was statistically significant. This was not the case for VbP alone (data not shown), indicating that the varied responses were due to activation of the prodrugs by FAPα.

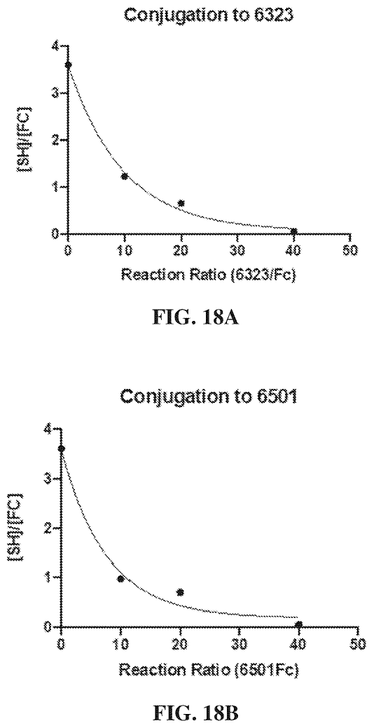

In an additional study, the conjugation of two prodrugs (6323 and 6501 (FIG. 6C)) to the hinge region cysteine residues of huIgG1 Fc was examined. Briefly, compound maleimide linker prodrugs (6323 and 6501) were dissolved in DMSO at a concentration of 100 mM. 6323 or 6501 were added to reduced Fc and the samples were incubated at room temperature. Unreacted 6323 or 6501 was removed with a Zeba spin column. Before measuring free thiol (SH) groups, the samples were reduced again and dithiothreitol (DTT) removed by passing samples over the Zeba spin desalting column again. Conjugation of the thiol groups was determined by measuring free SH groups using Ellman's reagent. The concentration of the reduced Fc samples was determined from the absorbance at 280 nm, using an extinction coefficient of 66500 M⁻¹ cm⁻¹. Conjugations were done with reaction ratios of 0, 10, 20, and 40 moles of 6323 or 6501 per mole of reduced SH (4 SH per Fc). The decreases in the number of free SH groups on Fc by conjugation to 6323 (FIG. 18A) or 6501 (FIG. 18B) is represented as the change in free SH groups vs. reaction ratio.

A study relating to the conjugation of 6323 to AFFIMER®-Fc proteins was undertaken. A compound maleimide linker prodrug (6323) was dissolved in DMSO at a concentration of 100 mM. The concentration of the reduced SQTGlyCF samples was determined from the absorbance at 280 nm. 6323 was added to the reduced SQTGlyCF and the samples were incubated at room temperature. Unreacted 6323 was removed with a Zeba spin column. Before measuring free SH groups, samples were reduced again as described above to ensure that any unreacted cysteines were reduced. Conjugation of the thiol groups was determined by measurement of free SH groups with using Ellman's reagent. The concentration of thiols was determined from the absorbance at 412 nm using an extinction coefficient of 14,150 M⁻¹ cm⁻¹.

The number of thiols per SQTGlyCF was calculated as the ratio of the thiol concentration to the protein concentration ([SH]/[SQTGlyCF]). Conjugations were done with reaction ratios of 0, 10, 20 and 40 moles of 6323 per mole of reduced SH (4 SH per SQTGlyCF). The change in free SH groups vs. reaction ratio is shown in FIG. 24A. A timed conjugation reaction was done with a reaction ratio of 40 6323 per SH with the reaction stopped at 0, 5, 10, 15 and 20 minutes after addition of 6323. Kinetics of conjugation of 6323 to SQTGlyCF with a reaction ratio of 40 6323 per SH group are shown in FIG. 24B.

### Example 5—Pharmacokinetic Studies (Tumor Mouse Models)

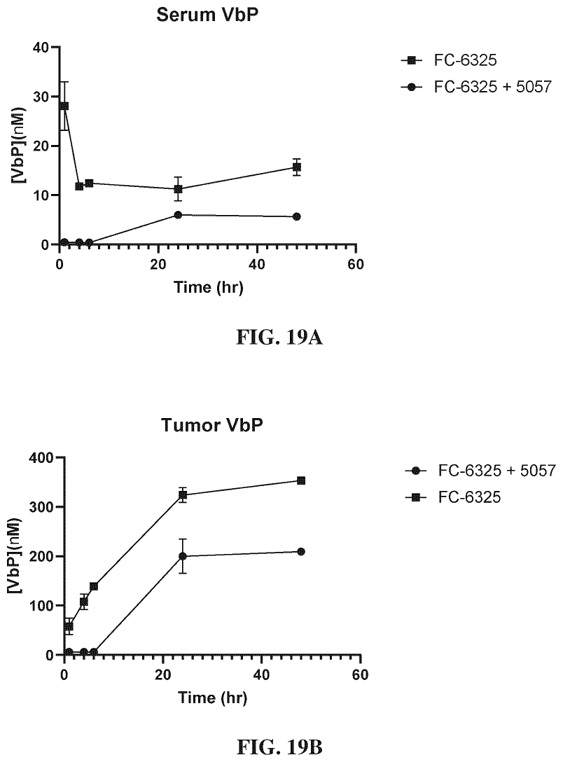

The pharmacokinetics of different compounds were examined in murine models of cancer. Mice (BALB/c) were inoculated subcutaneously in the right flank with 5×10⁵ CT26-mFAP cells. Tumors were allowed to become established, after which, animals were treated with or without 5057 (FAPα inhibitor) for 24 hours. Then, animals were treated with hu IgG1 Fc-6325 (Fc-6325, 200 μg/animal) for 1, 4, 6, 24 and 48 hours. At the designated time points, blood and tumor samples were collected to measure Val-boroPro (VbP) on LC-MS. Treatment with Fc-6325 resulted in higher concentrations of VbP in the tumor than in the serum at all time points. The FAPα inhibitor significantly reduced the levels of VbP, and the values of VbP in the tumor at 24 and 48 hours were greater than at 1-6 hours. It is thought that this was due to FAP inhibition waning during the 6-24 hour period. Concentrations of VbP (nM) over time (hours) for serum (FIG. 19A) or tumor (FIG. 19B) samples is shown. Each time point includes 3 mice per group.

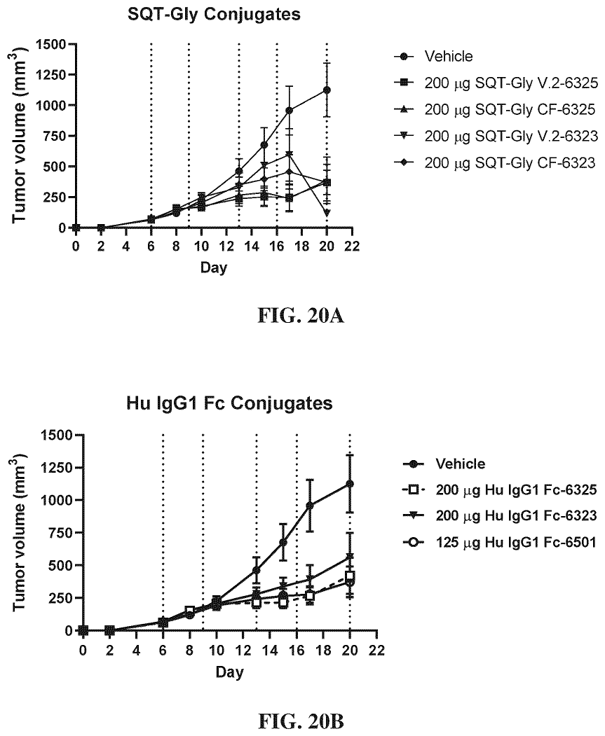

Next, conjugates of different compounds were tested. Mice (n=10/group, BALB/c) were inoculated subcutaneously in the right flank with 5×10⁵ CT26-mFAP cells. Conjugates were injected intraperitoneally once tumor volumes averaged approximately 50-100 mm³. Treatments were administered twice a week for 3 weeks. The change in tumor volume over time in CT26-mFAP+mice with either vehicle (control) or SQT-Gly conjugates (FIG. 20A) or Hu IgG1 Fc conjugates (FIG. 20B) is shown. In FIGS. 20A-20B, the dotted lines represent twice-weekly (BIW) dosing. The SQT-Gly conjugates tested were as follows: SQT-Gly V.2 (IgG1 Fc) conjugated with either 6325 (NHS) or 6323 (MAL) and SQT-Gly CF (IgG1 LALA Fc) conjugated with either 6325 or 6323. The hu IgG1 Fc conjugates tested were as follows: Hu IgG1 Fc fragment conjugated with either 6325 or 6323 or 6501 (a tetrameric prodrug; FIG. 6C). All mice treated with conjugates had a reduced tumor size compared to the vehicle control on Day 20. The efficacy of NHS (6325) or MAL (6323 or 6501) conjugates was comparable between the half-life extended versions of SQT-Gly and hu IgG1 Fc.

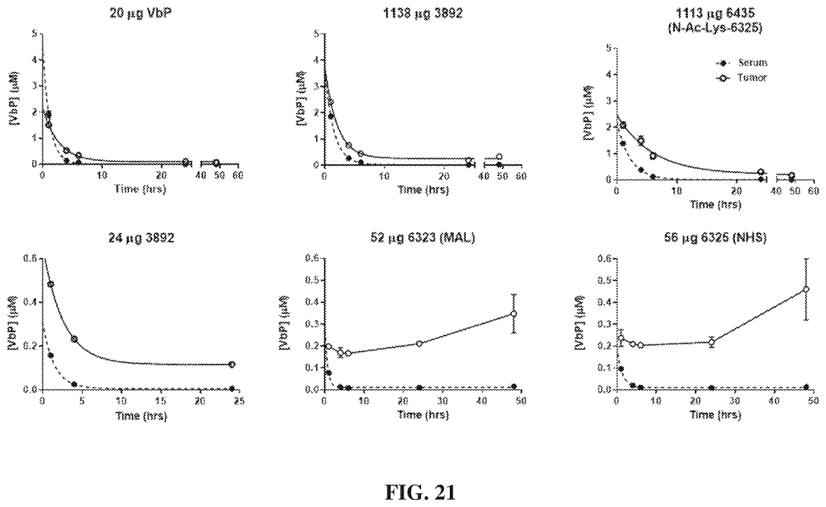

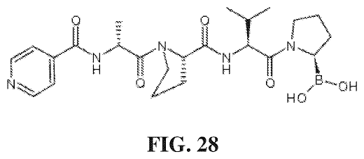

In addition, the pharmacokinetics and tissue distribution of VbP released from different FAP-activated prodrugs were examined. Mice (BALB/c) were inoculated subcutaneously in the right flank with 5×10⁵ CT26-mFAP cells. Tumors were allowed to become established, after which, animals were dosed via a 200 uL intraperitoneal injection of 24 μg 3892 (the structure of 3892 is shown in FIG. 28), 1138 μg 3892, 52 μg 6323, 56 μg 6325, or 1113 μg 6435 (N-Ac-Lys-6325). Each dosing group consisted of a subset of 5 groups, representing the 1, 4, 6, 24 and 48 hour collection time points (n=3/group) with the exception of 24 ug 3892, which only consisted of 1, 4 and 24 hours. At the collection time points, blood and tumor samples were collected to measure VbP levels on LC-MS. VbP concentration vs. time in serum and tumor following prodrug administration is represented in FIG. 21. The 1138 ug dose of 3892 and 1113 ug dose of 6435 (N-Ac-Lys-6325) represented 25× and 10× equivalents of released VbP in comparison to 20 ug VbP alone, respectively. Both exhibited serum and tumor distribution profiles similar to VbP alone, with peak concentrations occurring 1 hour after dosing, followed by a steady decline over time. The lower prodrug doses of 24 μg 3892, 52 μg 6325 and 56 μg 6325 were selected to represent the approximate contained VbP concentrations associated with the conjugates. Both 6323 and 6325 resulted in concentrations of approximately 200 nM tumor VbP that remained steady in the early time points, followed by an increase from 24 to 48 hours to 400 nM.

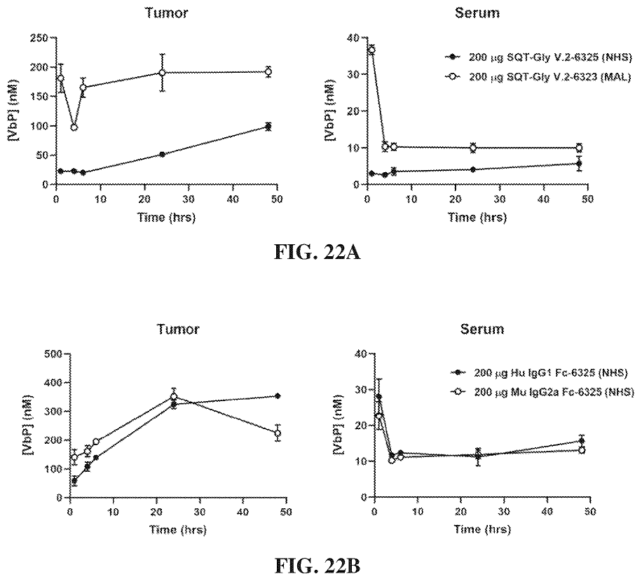

Next, the pharmacokinetics and tissue distribution of VbP released from SQT-Gly V.2-conjugates and IgG Fc-conjugates were examined. Mice (BALB/c) were inoculated subcutaneously in the right flank with 5×10⁵ CT26-mFAP cells. Tumors were allowed to become established, after which, animals were dosed via a (200 μg/200 uL) intraperitoneal injection of SQT-Gly V.2-6323, SQT-Gly V.2-6325, Mouse IgG2a Fc-6325, or Hu IgG1 Fc-6325. Each dosing group consisted of a subset of 5 groups, representing the 1, 4, 6, 24 and 48 hour collection time points (n=3/group). At the collection time points, blood and tumor samples were collected to measure released VbP levels using LC-MS. VbP concentration vs. time in serum and tumor following administration of SQT-Gly V.2-conjugates (FIG. 22A) or IgG Fc-conjugates (FIG. 22B) is shown.

The serum and tumor PK/TD profile of SQT-Gly V.2-6323 was assessed for comparison to SQT-Gly V.2-6325. While serum VbP levels for SQT-Gly V.2-6325 remained low (<10 nM) at all time points tested, SQT-Gly V.2-6323 resulted in a spike >10-fold higher than SQT-Gly V.2-6325 at 1 hour (37 vs. 3 nM), potentially indicating that the MAL conjugation sites increase accessibility for prodrug cleavage by serum FAP. Tumor VbP levels were also higher for SQT-Gly V.2-6323, which may further support increased prodrug activation of the MAL conjugate.

Mu IgG2a Fc-6325 was assessed for comparison to Hu IgG1 Fc-6325. There is a possibility that efficacy of the latter is not due solely to its design as an extended half-life prodrug, but also as the results of a secondary immune response triggered by the presence of a foreign antigen (Hu IgG1 Fc). As the Mu IgG2a Fc is of mouse origin, it was designed to bypass this issue. The PK/TD profiles of the two were quite similar. There was a reduction of VbP levels in the tumor observed for Mu IgG2a Fc-6325 between 24-48 hours that was not observed for Hu IgG1 Fc-6325; however, which may indicate that the half-life of the mouse conjugate is shorter than the human conjugate.

### Example 6-42CQ-Based Conjugates

The FAP-activated prodrug G-CSF serum cytokine response in nontumor-bearing BALB/c mice was examined. Female BALB/c mice of 11-12 weeks of age were injected intraperitonially with vehicle (PBS), 42CQ-6501, or 42CQ-6501+MSA at 200 μg/mouse, in groups of 5 mice per treatment.

VbP alone is known to elicit a large serum G-CSF response. While cytokine recruitment is believed to play an important role in tumor immunity, its presence in the serum represents a systemic response, which can lead to unwanted adverse effects. A panel of 42CQ-based conjugates were screened to assess whether they resulted in attenuated serum G-CSF responses in comparison to VbP. 42CQ is an AFFIMER® polypeptide designed to bind to serum albumin (SEQ ID NO: 133). Consequently, the 42CQ-based conjugates (with 6501) were prepared with or without prebinding to MSA to determine whether there was an evident difference between the two formulations.

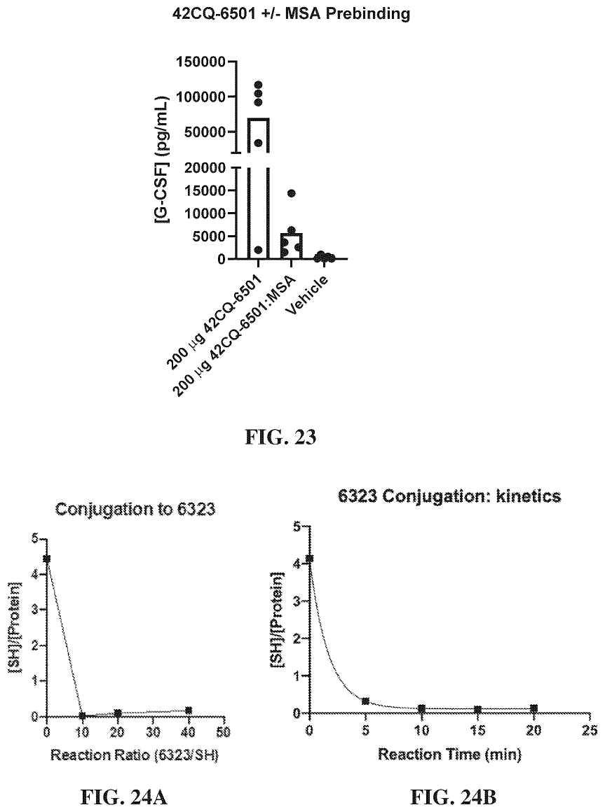

Six hours after dosing, blood was collected to measure G-CSF in the serum using a mouse G-CSF Quantikine ELISA kit. At the dose tested, 42CQ-6501 induced a significant increase in serum concentration of G-CSF compared to vehicle-treated mice, while prebinding 42CQ-6501 with MSA resulted in significant attenuation of serum concentration of G-CSF compared to 42CQ-6501 (FIG. 23).

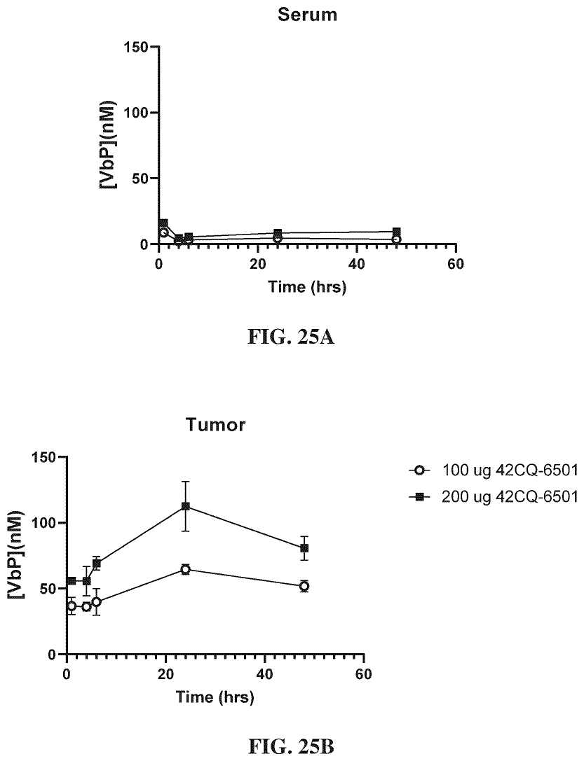

Next, the pharmacokinetics of the 42CQ-6501 conjugate in CT26-mFAP tumor-bearing mouse were examined. Briefly, BALB/c mice were inoculated subcutaneously in the right flank with 5×10⁵ CT26-mFAP cells. Tumors were allowed to become established, after which, animals were dosed via an intraperitoneal injection of 100 or 200 μg/animal of 42CQ-6501. Each dosing group consisted of a subset of 5 groups, representing the 1, 4, 6, 24 and 48 hour collection time points (n=3/group). At the collection time points, blood and tumor samples were collected to measure VbP levels on LC-MS. VbP concentration vs. time in serum (FIG. 25A) and tumor (FIG. 25B) following administration of 100 or 200 μg/animal of 42CQ-6501 are shown. Mean serum levels of VbP were between 4 and 15 times lower than tumor levels at all time points for both doses of 42CQ-6501.

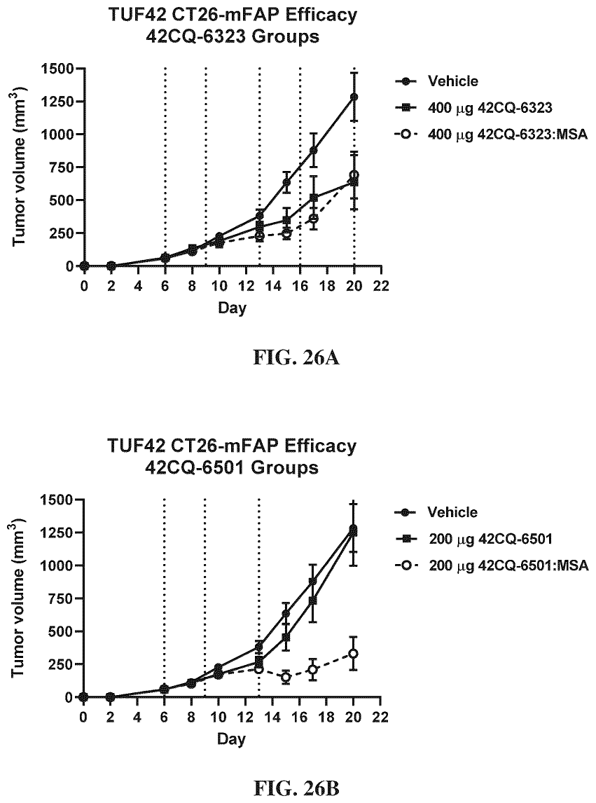

In a further experiment, the effect of 42CQ-based conjugates±MSA on tumor growth in a syngeneic murine colon cancer (CT26) model was examined. Mice (n=10/group, BALB/c) were inoculated subcutaneously in the right flank with 5×10⁵ CT26-mFAP cells. MSA prebinding may attenuate the systemic release of VbP, so the efficacy of both 42CQ-6323 and 42CQ-6501 conjugates was tested with and without MSA prebinding. The 42CQ-based conjugates±MSA were injected intraperitoneally once tumor volumes averaged approximately 50-100 mm³. Tumor growth inhibition for 42CQ-6323±MSA and 42CQ-6501±MSA is shown in FIGS. 26A and 26B, respectively. Dotted lines represent BIW dosing. All mice treated with 42CQ-based conjugates, except 42CQ-6501 without MSA, had a reduced tumor size compared to the control.

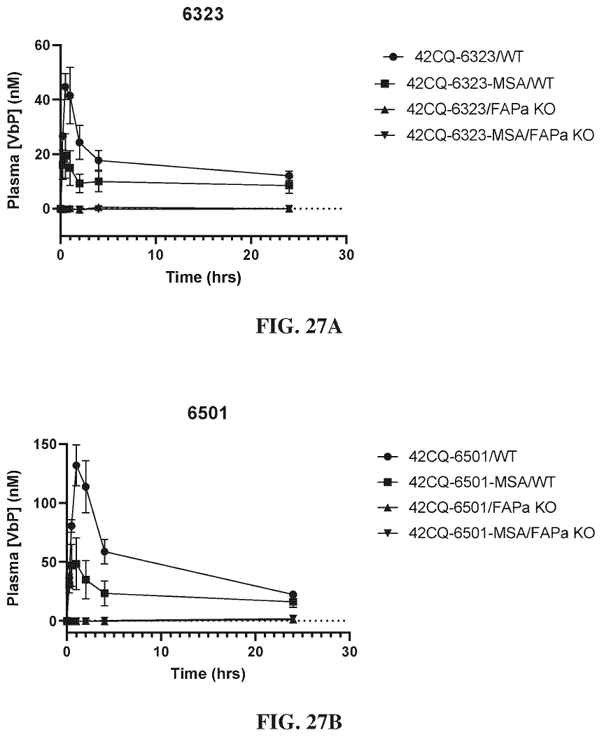

In an additional experiment, the pharmacokinetics of VbP released from FAPα-activated AFFIMER® prodrugs (42CQ-6323 and 42CQ-6501) in wild-type (WT) and FAPα knockout (KO) mice was examined. Non-tumor bearing WT or FAPα KO mice (n=3/group) were dosed via an intraperitoneal injection of 200 μg/animal of 42CQ-6323, 42CQ-6323-MSA, 42CQ-6501, or 42CQ-6501-MSA. Blood was collected at 0 (pre-dose), 15, 30, 60, 120, and 240 minutes, and at 24 hours post-dose to measure the concentration of VbP in plasma on LC-MS. VbP concentration vs. time in plasma following administration of 200 μg/animal of 42CQ-6323±MSA or 42CQ-6501±MSA is represented in FIGS. 27A and 27B, respectively.

No VbP was detected in the FAPα knockout mice at any timepoint. VbP levels in plasma were greater for 6501 conjugates than for 6323 conjugates, consistent with the greater effective drug-AFFIMER® protein ratio (DAR) for the 6501 conjugates. Pre-binding to albumin before injection appears to limit the initial spike of VbP in the blood (compare MSA groups to non-MSA groups).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A therapeutic conjugate comprising an acutely toxic drug moiety linked through an enzyme-cleavable linker to a half-life extension moiety, wherein the circulating serum half-life of the therapeutic conjugate in vivo is at least 48 hours, the conjugate does not comprise a cell-binding moiety that binds to a cell surface protein of a cell with a K_{d} of 1×10⁻⁶ M or less.

2. A therapeutic conjugate comprising an acutely toxic drug moiety linked through an enzyme-cleavable linker to a half-life extension moiety, wherein the circulating serum half-life of the therapeutic conjugate in vivo is extended by more than 2-fold relative to circulating serum half-life of a free drug moiety, and the conjugate does not comprise a cell-binding moiety that binds to a cell surface protein of a cell with a K_{d} of 1×10⁻⁶ M or less.

3. The therapeutic conjugate of claim 1 or 2, wherein the half-life extension moiety comprises a serum protein.

4. The therapeutic conjugate of claim 3, wherein the serum protein is selected from fibronectin, transferrin, and human serum albumin (HSA).

5. The therapeutic conjugate of any one of claims 1-4, wherein the half-life extension moiety comprises a molecule that binds to a serum protein, optionally selected from fibronectin, transferrin, and HSA.

6. The therapeutic conjugate of claim 5, wherein the molecule that binds to a serum protein is an antibody, optionally selected from the group consisting of: Fab, F(ab)₂, F(ab′), F(ab′)₂, F(ab′)₃, Fd, Fv, disulfide linked Fv, dAb or sdAb (or NANOBODY®), CDR, scFv, (scFv)₂, di-scFv, bi-scFv, tascFv (tandem scFv), AVIBODY® (e.g., diabody, triabody, and tetrabody), T-cell engager (BiTE®), Fc, scFv-Fc, Fcab, mAb₂, small modular immunopharmaceutical (SMIP), Genmab/unibody or duobody, V-NAR domain, IgNAR, minibody, IgGACH2, DVD-Ig, probody, intrabody, and a multispecificity antibody.

7. The therapeutic conjugate of claim 5, wherein the molecule that binds to a serum protein is a non-antibody molecule, optionally selected from the group consisting of: an affibody, an AFFIMER® polypeptide, an affilin, an anticalin, an atrimer, an avimer, a DARPin, an FN3 scaffold (e.g. Adnectins, Centyrins), a fynomer, a Kunitz domain, a nanofitin, a pronectins, a tribody, bicyclic peptides, and a Cys-knot.

8. The therapeutic conjugate of claim 7, wherein the half-life extension moiety comprises an HSA-binding recombinantly engineered variant of stefin polypeptide (i.e., AFFIMER® polypeptide).

9. The therapeutic conjugate of claim 8, wherein the recombinantly engineered variant of stefin polypeptide (AFFIMER® polypeptide) comprises an amino acid sequence that has at least 70%, at least 80%, at least 90%, or 100% identity to an amino acid sequence of any one of SEQ ID NOS: 110-132.

10. The therapeutic conjugate of claim 1 or 2, wherein the half-life extension moiety comprises an antibody Fc domain, optionally from IgA, IgD, IgE, IgG, or IgM or a subclass thereof.

11. The therapeutic conjugate of claim 1 or 2, wherein the half-life extension moiety comprises a biocompatible polymer, optionally selected from the group consisting of a poly(ethylene glycol) (PEG), a hydroxyethyl starch, an XTEN™ polymer, and a proline-alanine-serine polymer.

12. The therapeutic conjugate of any one of the preceding claims represented by one of the formula:
X-L¹-SRS-L²-DM, wherein
X is the half-life extension moiety,
L¹ is a spacer or bond,
SRS is a substrate recognition sequence cleavable by an enzyme,
L² is a self-immolative linker or bond, and
DM is the drug moiety.

13. The therapeutic conjugate of any one of the preceding claims represented by one of the formula:
X-(L1-SRS-L2-DM)n;
X-L1-(SRS-L2-DM)n;
(X)m-(L1-SRS-L2-DM)n; or
(X)m-L1-(SRS-L2-DM)n
wherein
X is the half-life extension moiety,
L¹ is a spacer or bond,
SRS is a substrate recognition sequence cleavable by an enzyme,
L² is a self-immolative linker or bond,
DM is the drug moiety,
m is an integer from 1 to 6, and
n is an integer from 1 to 500, optionally 1 to 100, 1 to 10, or 1 to 5.

14. The therapeutic conjugate of any one of the preceding claims, wherein the enzyme-cleavable linker is present extracellularly in a diseased tissue, optionally a cancerous tissue.

15. The therapeutic conjugate of any one of the preceding claims, wherein the enzyme-cleavable linker is an oligopeptide.

16. The therapeutic conjugate of claim 15, wherein the oligopeptide comprises a C-terminal proline covalently linked to the drug moiety, optionally via a bond or a self-immolative linker, and/or an N-terminal blocking group.

17. The therapeutic conjugate of claim 16, wherein the bond can be cleaved by the proteolytic activity of the enzyme, optionally wherein the bond is an amide bond.

18. The therapeutic conjugate of any one of claims 12-17, wherein the self-immolative linker comprises a heterocyclic self-immolative moiety, optionally His-Ala, p-aminobenzyloxycarbonyl (PABC) or and 2,4-bis(hydroxymethyl)aniline.

19. The therapeutic conjugate of any of the preceding claims, wherein the substrate recognition sequence is cleaved by a protease.

20. The therapeutic conjugate of any of the preceding claims, wherein the protease is a serine protease, metal protease, or cysteine protease.

21. The therapeutic conjugate of claim 20, wherein the protease is present extracellularly in the cancerous state tissue in a subject at levels at least 2, 3, 4, 5, 6, 7, 8, 9, 10, 15, 20, 30, 40, 50, 60, 70, 80, 90, or 100 times greater than the healthy state of the tissue in the subject.

22. The therapeutic conjugate of claim 20 or 21, wherein the protease is present extracellularly in the cancerous state of the tissue in a subject at levels at least 2, 3, 4, 5, 6, 7, 8, 9, 10, 15, 20, 30, 40, 50, 60, 70, 80, 90 or 100 times greater than other tissue of the subject.

23. The therapeutic conjugate of any of the preceding claims, wherein the protease is a matrix metalloproteinase selected from membrane bound matrix metalloproteinases (such as MMP14-17 and MMP24-25) and secreted matrix metalloproteinases (such as MMP1-13 and MMP18-23 and MMP26-28).

24. The therapeutic conjugate of claim 23, wherein the protease is selected from the group consisting of MMP1, MMP2, MMP3, MMP4, MMP9, MMP11, MMP13, MMP14, MMP17, and MMP19.

25. The therapeutic conjugate of any of the preceding claims, wherein the protease is an A Disintegrin and Metalloproteinase (ADAM), or an A Disintegrin or Metalloproteinase with Thrombospondin Motifs (ADAMTS).

26. The therapeutic conjugate of any of the preceding claims, wherein the protease is selected from the group consisting of a legumain, a matriptase (MT-SP1), a neutrophil elastase, a TMPRSS, a thrombin, a u-type plasminogen activator (uPA, also referred to as urokinase), PSMA, and CD 10 (CALLA).

27. The therapeutic conjugate of any one of the preceding claims, wherein the drug moiety induces an innate immune response in vivo.

28. The therapeutic conjugate of any one of the preceding claims, wherein the drug moiety is a cytotoxic agent.

29. The therapeutic conjugate of any one of the preceding claims, wherein the drug moiety is a cytostatic agent.

30. The therapeutic conjugate of any one of the preceding claims, wherein the drug moiety is an epigenetic agent.

31. The therapeutic conjugate of any one of the preceding claims, wherein the drug moiety is selected from the group consisting of TLR agonists, RIG-I agonists, iDASH inhibitors, and STING agonists.

32. The therapeutic conjugate of claim 31, wherein the drug moiety is a TLR agonist, such as a selected from the group consisting of a TLR1/2 agonist, a TLR2 agonist, a TLR3 agonist, a TLR4 agonist, a TLR5 agonist, a TLR6/2 agonist, a TLR7 agonist, a TLR7/8 agonist, a TLR7/9 agonist, a TLR8 agonist, a TLR9 agonist, and a TLR11 agonist, preferably selected from the group consisting of a TLR3 agonist, a TLR7 agonist, a TLR7/8 agonist, and a TLR9 agonist.

33. The therapeutic conjugate of claim 31, wherein the drug moiety is an iDASH inhibitor that inhibits the enzymatic activity of DPP8 and DPP9 and induces macrophage pyroptosis.

34. The therapeutic conjugate of any one of claims 1-28, wherein the drug moiety is Val-boroPro (Talabostat).

35. The therapeutic conjugate of any one of the preceding claims, wherein the drug moiety comprises a ligand receptor.

36. The therapeutic conjugate of claim 35, wherein the ligand receptor is selected from the group consisting of: somatostatin, cholecystokinin-2 (CCK2), folate, bombesin, gastrin-releasing peptide, neurotensin, substance P, glucagon-like peptide 1, neuropeptide Y, and analogs thereof.

37. The therapeutic conjugate of any one of the preceding claims, wherein the drug moiety is represented by:
—RBM [symbol] Z)ₚ
wherein RBM is a receptor binding moiety, Z is cytotoxic, cytostatic or epigenetic moiety or a radioisotope containing moiety, and p is 0 (Z is absent) or an integer from 1 to 8.

38. The therapeutic conjugate of claim 37, wherein p is 1.

39. The therapeutic conjugate of any one of the preceding claims, wherein the drug moiety is represented by:
—RBM-L³-Z
wherein RBM is a receptor binding moiety, Z is cytotoxic, cytostatic or epigenetic moiety or a radioisotope containing moiety, and L³ is a bond or a cleavable or non-cleavable linker.

40. The therapeutic conjugate of claim 39, wherein L³ comprises an acid labile linker or an enzyme-sensitive linker.

41. The therapeutic conjugate of claim 30, wherein the linker comprises a cathepsin cleavage site.

42. The therapeutic conjugate of any one of the preceding claims, wherein the drug moiety has a circulating serum half-life that it is at least 5, 10, 25, 50, 100 or even 1000 times longer than the circulating serum half-life of the free drug moiety.

43. The therapeutic conjugate of any one of the preceding claims, wherein the drug moiety has a circulating serum half-life that it is at least 10, 25, 50, 100, 200, or 300 hours.

44. The therapeutic conjugate of any one of the preceding claims, wherein for a period of at least 10, 24, 48, 72, 96, or 120 hours the therapeutic conjugate produces a concentration of free drug moiety in target tissue expressing the enzyme that is at least 2, 5, 10, 20, 50, 75 or 100 times the concentration of free drug moiety in systemic circulation over the same period of time.

45. The therapeutic conjugate of any one of the preceding claims, wherein when administered to a subject having a tumor expressing the enzyme, the therapeutic conjugate produces an intratumoral concentration of free drug moiety that is at or above the EC₅₀ for the antitumor activity of the free drug moiety for a period at least 10, 24, 48, 72, 96, or 120 hours.

46. The therapeutic conjugate of any one of the preceding claims, wherein when administered to a subject having a tumor expressing the enzyme, the therapeutic conjugate has a therapeutic index for antitumor activity of at least 2, 5, 10, 25, 50, 100, or 500.

47. The therapeutic conjugate of any one of the preceding claims, wherein when administered to a subject having an tumor expressing the enzyme, the therapeutic conjugate has a therapeutic index for antitumor activity that is at least 2, 10, 50, 100, 250, 500, 1000, 5000, or 10,000 times greater than the therapeutic index of the free drug moiety.

48. The therapeutic conjugate of any one of the preceding claims, wherein a greater percentage of free drug moiety is localized in a target tissue expressing the enzyme, relative to free drug moiety, when compared on an equivalent dose basis, optionally wherein the ratio of free drug moiety localized in the target tissue relative to other tissue (such as blood, liver or heart) is at least 2, 5, 10, 100, or 1000 times greater for an equivalent dose of the therapeutic conjugate relative to the free drug moiety.

49. The therapeutic conjugate of any one of the preceding claims, wherein the maximum tolerated dose of the therapeutic conjugate is at least 2, 5, 10, 100, or 1000 times greater than the maximum tolerated dose of the free drug moiety.

50. The therapeutic conjugate of any one of the preceding claims, wherein the cell permeability of the therapeutic conjugate is at least 50%, 60%, 70%, 80%, 90%, 95%, 98%, 99%, or 99.9% less than the cell permeability of free drug moiety.

51. The therapeutic conjugate of any one of the preceding claims, wherein the circulating half-life of the therapeutic conjugate is at least 25%, 50%, 75%, 100%, 150%, 200%, 500%, 750%, or 1000% longer than the circulating half-life of free drug moiety.

52. The therapeutic conjugate of any one of the preceding claims, wherein the therapeutic conjugate has less than 50%, 60%, 70%, 80%, 90%, 95%, 98%, 99%, 99.9%, or 99.99% of the cytotoxic or cytolytic activity against tumor cells relative to free drug moiety.

53. A composition comprising the therapeutic conjugate of any one of the preceding claims and a pharmaceutically acceptable excipient.

54. A method comprising administering to a subject the therapeutic conjugate or composition of any one of the preceding claims, wherein the subject has a diseased tissue, optionally a cancer.

55. The method of claim 54, wherein the therapeutic conjugate or composition is administered in an amount effective to increase lactate dehydrogenase (LDH) release in a tumor microenvironment the subject by at least 0.5-fold or at least 1-fold relative to an untreated control subject.

56. The method of claim 54 or 55, wherein the volume of the diseased tissue, optionally a tumor, is reduced by at least 50%, at least 60%, or at least 70% at about 2-3 weeks following administration of the therapeutic conjugate or composition.

57. The therapeutic conjugate or composition of any one of the preceding claims for use in a method for treating a diseased tissue, optionally a cancer.

58. Use of the therapeutic conjugate of any one of the preceding claims in the manufacture of a medicament for the treatment of a diseased tissue, optionally a cancer.
